SEQUENCE LISTING (627 sequences)

SEQ ID NO: 1 - protein, 75 aa, Acinetobacter baumannii
MPFINIQTIEGLLDKDSKAELFKRITDLFVEIEGKGNPAFREHVWIRIDEYPPEQWQLGSLSPTKQMIELMTASK

SEQ ID NO: 2 - protein, 61 aa, Bacillus cereus
MPYVTVKMLEGRTEEQKKALAEKVTAAVSETTGAPEENIIVFIEEMSKNHYAVGGKRLSDK

SEQ ID NO: 3 - protein, 65 aa, Bartonella elizabethae
MPYVNIKITNEDVTSEQKRQLIEGATQLLVDVLNKNPRTTVVVIDEVDTDNWGIGGVPVTELRKL

SEQ ID NO: 4 - protein, 62 aa, Bordetella sp. FB-8
MPYIRVEMLEGRSDEQKAKLAQAITNAMVEHAGAKPDSIFVVIEDVKKSNWATGGTLMSQRK

SEQ ID NO: 5 - protein, 78 aa, Burkholderia sp. RPE67
MPIVTIQVTREGNTPGANAVTADEKAQLIKGVSELLRDVLNKPLDSTFVVIQEVELENWGWGGLPVPEYRRQKQARDS

SEQ ID NO: 6 - protein, 69 aa, Campylobacter jejuni
MPFVNIRITKENGKPTTEQKQELIAGVTDLLAKVLNKNKSSTVVIIDEIDTDNYGLGGKSITQVRKEKS

SEQ ID NO: 7 - protein, 75 aa, Citrobacter freundii
MPHVDIKCFPRDLNEEEKAALATDITEVIIRHLQSKESSVSVALNEVEPDAWQAVWDAEIAPQMTRLIKKPGYSM

SEQ ID NO: 8 - protein, 76 aa, Enterobacter cloacae
MPHVDIKCFPRDLNDEQKTALAADIAEVIIRHFNSKDGSVSVALNQVNPEDWKAQVWDTEIGPKLDELIKKPGYSM

SEQ ID NO: 9 - protein, 75 aa, Escherichia coli
MPHIDIKCFPRELDEQQKAALAADITDVIIRHLNCKDSSISIALQQIQPESWQAIWDAEIAPQMEALIKKPGYSM

SEQ ID NO: 10 - protein, 68 aa, Haemophilus parainfluenzae
MPYVNIKVTGGAEAPTAKQKAELIEGVTHLLQKVLNKNPETTVVVIDEVDMDNWGIGGKTVTSRRSMK

SEQ ID NO: 11 - protein, 68 aa, Helicobacter pylori
MPFINIKLVPENGGSTNEQKQQLIEGVSDLMVKVLNKNKASIVVIIDEVDSNNYGLGGESVYHLRQKN

SEQ ID NO: 12 - protein, 74 aa, Leptospira interrogans
MPLVQIFVPAGSLSADMKNDMIQKVTDAVVETEGKPVVRRYTWVHINEVPDGGWGMSGKVVTIDAMKKSLEKTE

SEQ ID NO: 13 - protein, 65 aa, Mycobacterium intracellulare
MPLVEVTLVQGRAPHQLRTLISELTDAVETALGVSRSAIRVVLREVPDTHWAAGDVTIAERNKSS

SEQ ID NO: 14 - protein, 69 aa, Neisseria meningitidis
MPYVNIKVTGGKEAPTAAQKAELIGGVTELLARVLGKNPETTVVVIDEVDTDNWGIGGKSVSERRKEGR

SEQ ID NO: 15 - protein, 69 aa, Neisseria gonorrhoeae
MPYVNIKVTGGKEAPTAAQKAELIGGVIELLARVLGKNPETTVVVIDEVDTDNWDIGGKSVSERRKEGR

SEQ ID NO: 16 - protein, 71 aa, Pseudomonas aeruginosa
MPYVNIRVTREGVSADQKRQLIEGATDLLLKVLGKPPESTFVVIDEVDPDNWGVAGESVSALRRRQTPRTS

SEQ ID NO: 17 - protein, 63 aa, Pseudomonas putida
MPIAQLYILEGRSDEQKESLIREISEAMSRSLDAPIERVRVIITEMPKNHFGIGGEPASKLNR

SEQ ID NO: 18 - protein, 71 aa, Pseudomonas aeruginosa
MPLVTVKGIEGVFSTEQKAEIISKITDAMVSVEGEKMRGVTWVVFEEVKSGDWGIGGEPITTEKVRALQNS

SEQ ID NO: 19 - protein, 77 aa, Salmonella enterica
MPHVDIKCFPRDLTDEAKLALAADITDVIIRHLQSKESSISVALNHVPPEEWQAVWDTEIAPQMETLIKKPGYTMSK

SEQ ID NO: 20 - protein, 61 aa, Staphylococcus aureus
MPIVNVKLLEGRSDEQLKNLVSEVTDAVEKTTGANRQAIHVVIEEMKPNHYGVAGVRKSDQ

SEQ ID NO: 21 - protein, 60 aa, Streptococcus pneumoniae
MPFVRIDLFEGRTLEQKKALAKEVTEAVVRNTGAPRSAVHVIINDMPEGTYFPQGEMRTK

SEQ ID NO: 22 - protein, 60 aa, Streptococcus agalactiae
MPFVKIDLFEGRSQEQKNELAREVTEVVSRIAKAPKENIHVFINDMPEGTYYPQDELKKK

SEQ ID NO: 23 - protein, 60 aa, Streptococcus suis
MPFVRIDLFEGRTEEQKIALAREVTEVVSRNTNAPKEAIHVFINDMPEGTYYPQGEMKRK

SEQ ID NO: 24 - protein, 64 aa, Vibrio fluvialis
MPYINVKVTDDGVTKEQKQAIIKGCTQLMVDILNKNPEKTFVVIDEVNTDNWGVGFDQVTELRR

SEQ ID NO: 25 - protein, 66 aa, Yersinia pseudotuberculosis
MPYVNIKITREGATAEQKKQLIAGVTQLLVDTLGKNPATTVVVIDEVDTDNWGIGGRSVTDLRQSS

SEQ ID NO: 26 - protein, 66 aa, Yersinia enterocolitica
MPYVNIKITREGATAEQKKQLIAGVTQLLVDTLGKNPATTVVVIDEVDTDNWGIGGHSVTELRKSS

SEQ ID NO: 27 - protein, 62 aa, Artificial Sequence
  SAR1376
MMPIVNVKLLEGRSDEQLKNLVSEVTDAVEKTTGANRQAIHVVIEEMKPNHYGVAGVRKSDQ

SEQ ID NO: 28 - protein, 62 aa, Artificial Sequence
  SAR1376 P1A
MMAIVNVKLLEGRSDEQLKNLVSEVTDAVEKTTGANRQAIHVVIEEMKPNHYGVAGVRKSDQ

SEQ ID NO: 29 - protein, 62 aa, Artificial Sequence
  SAR1376 R35A
MMPIVNVKLLEGRSDEQLKNLVSEVTDAVEKTTGANAQAIHVVIEEMKPNHYGVAGVRKSDQ

SEQ ID NO: 30 - DNA, 192 bp, Artificial Sequence
  SAR1376
atgatgcccatcgtgaacgtgaagctgctggaaggcagaagcgacgagcagctgaagaacctggtgtccgaagtgaccgacgccgtggaaaagaccaccggcgccaacagacaggccatccacgtcgtgatcgaggaaatgaagcccaaccactacggcgtggccggcgtgcggaaaagcgatcagtgatga

SEQ ID NO: 31 - DNA, 192 bp, Artificial Sequence
  SAR1376 P1A
atgatggccatcgtgaacgtgaagctgctggaaggcagaagcgacgagcagctgaagaacctggtgtccgaagtgaccgacgccgtggaaaagaccaccggcgccaacagacaggccatccacgtcgtgatcgaggaaatgaagcccaaccactacggcgtggccggcgtgcggaaaagcgatcagtgatga

SEQ ID NO: 32 - DNA, 192 bp, Artificial Sequence
  SAR1376 R35A
atgatgcccatcgtgaacgtgaagctgctggaaggcagaagcgacgagcagctgaagaacctggtgtccgaagtgaccgacgccgtggaaaagaccaccggcgccaacgcccaggccatccacgtcgtgatcgaggaaatgaagcccaaccactacggcgtggccggcgtgcggaaaagcgatcagtgatga

SEQ ID NO: 33 - DNA, 1107 bp, Artificial Sequence
  SA1388
atgaagatcgccgacctgatgaccctgctggaccaccacgtgccctttagcacagccgagagctgggacaacgtgggcctgctgattggcgacggcgacgtggaagtgaccggcgtgctgacagccctggactgcacactggaagtcgtgaacgaggccatcgagaagggctacaacaccatcatcagccaccaccccctgatcttcaagggcgtgaccagcctgaaggccaacggctacggcctgatcatccggaagctgatccagcacgacatcaacctgatcgccatgcacaccaacctggacgtgaacccctacggcgtgaacatgatgctggccaaggccatgggcctgaagaacatcagcatcatcaacaaccagcaggacgtgtactacaaggtgcagacctacatccccaaggataatgtgggccccttcaaggacaagctgagcgagaatggcctggcccaggaaggcaactacgagtactgcttcttcgagagcgagggcagaggccagttcaagcctgtgggcgaggccaaccctaccatcggccagatcgacaagatcgaggacgtggacgaagtgaagatcgagttcatgatcgacgcctaccagaagtccagagccgagcagctgatcaagcagtaccacccctacgagacacccgtgttcgacttcatcgagattaagcagacctccctgtacggcctgggcgtgatggccgaggtggacaaccagatgactctggaagatttcgccgccgacatcaagagcaagctgaacatcccctccgtcagattcgtgggcgagagcaaccagaagatcaagcggatcgccatcatcggcggcagcggcatcggctacgagtatcaggctgtgcagcagggcgccgacgtgttcgtgacaggcgatatcaagcaccacgacgccctggacgccaagatccatggcgtgaacctgatcgacatcaaccactacagcgagtacgtgatgaaggaaggcctgaaaaccctgctgatgaactggttcaatatcgagaagattaacattgatgtggaagccagcaccatcaataccgaccccttccagtacatctgatgatga

SEQ ID NO: 34 - DNA, 450 bp, Artificial Sequence
  Dps
atgagcaaccagcaggacgtcgtgaaagaactgaatcagcaggtggccaactggaccgtggcctacaccaagctgcacaacttccattggtacgtgaagggccccaacttcttcagcctgcacgtgaagttcgaggaactgtacaacgaggccagccagtacgtggacgagctggccgagagaatcctggccgtgggcggaaatcctgtgggcaccctgaccgagtgcctggaacagagcattgtgaaagaggccgccaagggctacagcgccgagcagatggtggaagaactgagccaggacttcaccaacatcagcaagcagctggaaaacgccatcgagatcgccggcaacgctggcgacgatgtgtccgaggacatgttcatcggcatgcagaccagcgtggacaagcacaactggatgttcaagagctacctgagctgatgatga

SEQ ID NO: 35 - DNA, 570 bp, Artificial Sequence
  QacR
atgaacctgaaggacaagatcctgggcgtggccaaagagctgttcatcaagaacggctacaacgccaccaccaccggcgagatcgtgaagctgagcgagagcagcaagggcaacctgtactaccacttcaagaccaaagagaacctgttcctggaaatcctgaacatcgaggaatccaagtggcaggaacagtggaagaaagaacagatcaagtgcaagaccaaccgcgagaagttctacctgtacaacgagctgagcctgaccaccgagtactactaccccctgcagaacgccatcatcgagttctgcacagagtactacaagaccaatagcatcaacgagaagatgaacaagctggaaaacaagtacatcgacgcctaccacgtgatcttcaaagagggcaatctgaacggcgagtggtgcatcaatgacgtgaacgccgtgtccaagatcgccgccaacgccgtgaatggcatcgtgaccttcacccacgagcagaacatcaatgagcggatcaagctgatgaacaaattcagccagatcttcctgaacggcctgagcaagtgatga

SEQ ID NO: 36 - protein, 4 aa, Artificial Sequence
  Linker
SGSG

SEQ ID NO: 37 - protein, 14 aa, Artificial Sequence
  V5 epitope tag
GKPIPNPLLGLDST

SEQ ID NO: 38 - protein, 69 aa, Ponticaulis koreensis
MPFVNIKVTREGVTAEQKAQLIEGVTTLLKDVLNKNPSTTFVVIDEVDLDNWGAGGLPVTEFRKRALNT

SEQ ID NO: 39 - protein, 68 aa, Campylobacter jejuni
MPIVNIKLAKPALSKEQKAELIADVSELLSTKYHKAKERVVVMIEDIEPYDIGFGGESVEAIKAKANK

SEQ ID NO: 40 - protein, 61 aa, Listeria monocytogenes
MPFVTIQFLEGRTDDQKKALVSEVTEVVSKNLKAPKENIHVILEEMKKTDYGVGGVRKSDI

SEQ ID NO: 41 - protein, 72 aa, Pseudomonas sp. CFII68
MPFANYKFPEGILDHARKEEIIHRTTEMFVEYFGEGVRPFTMVLVEEVADGGWGRADETLTLAKMGLPAKGQ

SEQ ID NO: 42 - protein, 64 aa, Brevibacillus brevis
MPYINLQITKGATRDQKAQIVKEFTETLVNVLGKKPEHTHIVIEEIDDENWGYAGILTDDFRKL

SEQ ID NO: 43 - protein, 76 aa, Enterobacter cloacae
MPHVDIKCFPRDLNDEQKTALAADIADVIIRHFNSKDSSVSVALNQVAPEDWKAEVWDTEIGPKLDELIKKPGYSM

SEQ ID NO: 44 - protein, 59 aa, Leptospira fainei
MPYVNVKVAGPLTKEQKQTIVKEFTETLRKVAAKPPETTYIVIDEVSRENWAAGGKLLE

SEQ ID NO: 45 - protein, 78 aa, Dickeya zeae
MPFVNVQTIKGILSPEQKSELLRRMTDLIVDIEGKGDPEFRRSVWIRIDEHDPEQWSLGGIQPTSEMIAAKFASPQRS

SEQ ID NO: 46 - protein, 70 aa, Coleofasciculus chthonoplastes
MPFVTVQIAKGHSVDQKRELAKALTDTLVSTLGTKPEWVTIHIDEFERENWAVGGELHSDKPGKGGKHHT

SEQ ID NO: 47 - protein, 68 aa, Kaistia granuli
MPFINIRLVREVIASDPEGKKATIAAKVTEAVKEATGLSDSDVWVVFEEVAARDWYLGPKSVAARRGL

SEQ ID NO: 48 - protein, 65 aa, Halotalea alkalilenta
MPVVRVSWFEGKDAKAKQAVAAEITESIVKNTGTDPNYIYVIFEDVKPSDWAGAGKLFGEDPESA

SEQ ID NO: 49 - protein, 71 aa, Pseudomonas aeruginosa; identical to SEQ ID NO: 18

SEQ ID NO: 50 - protein, 77 aa, Sphingobium indicum
MPFVDIRLAGNATREQKAAIVADVTRSLVERLGKSPAAVQVVISEVSTENYGAGGQLIADRDAPKAGEDAHAAVTSR

SEQ ID NO: 51 - protein, 63 aa, Neisseria wadsworthii
MPIIQVHMMEGRTDEQKEAVIREVTEAMVRAVGAPKESVRVLINEMPKQHFGIGGESAKAKGR

SEQ ID NO: 52 - protein, 69 aa, Campylobacter jejuni
MPFVNIRITKENEQPTKEQKQELIAGITDLLAKILNKNKNSTVVIIDEIDTDNYGFGGKSITQVRQEKS

SEQ ID NO: 53 - protein, 60 aa, Streptococcus cristatus
MPFVRIDLFEGRSLEQKKALAKEVTEAVVRNTGAPQSAVHVIINDMPEGTYFPQGEMRTK

SEQ ID NO: 54 - protein, 67 aa, Campylobacter concisus
MPYVNIKIAGPEPTKEQKDQVFKEVTETLVRVLGKKKEAVMIFIETHDAGNIGVGGESVEDKRKGIK

SEQ ID NO: 55 - protein, 61 aa, Janthinobacterium sp. CG3
MPTINVQLFEGRTLEQKRAFVQAVTEATVKTLGAAPESVDILIHEIKREHWATGGKLWSDE

SEQ ID NO: 56 - protein, 67 aa, Acinetobacter sp. NIPH 236
MPYILIQITKENVTTAQKQQLIQEATDLVVRVLHKDPATTFVVIDEIETDNWGVAGESVSTIRNRQR

SEQ ID NO: 57 - protein, 78 aa, Dyella japonica
MPIVNIQITREGTGPGRHAATAEEKAAVIKGVSQVLLDVLKKPLDATFVVIEEVELENWGWGGQSVSALRRQQAGGSR

SEQ ID NO: 58 - protein, 64 aa, Vibrio coralliilyticus
MPVVTIQQSPGRTKEQKQLLIKRITEAFEEAYGMPPEGVTVFFQNFDDEHWGKGGYLHADRDVT

SEQ ID NO: 59 - protein, 59 aa, Corynebacterium sputi
MPLIQISQTPGLDAEQKKAIIESVTKVYAEVSGKKESSVWVTINEIERDSWGVGGVPLG

SEQ ID NO: 60 - protein, 70 aa, Salinispora arenicola
MPFANLKVPAGTLSAEQKETLVAKVTDLYVDTFGDAVRDNTMVLVDEVVDGGWGLRGDVLTLAKIQQRSG

SEQ ID NO: 61 - protein, 71 aa, Anaerotruncus sp. G3(2012)
MPYIKVTGGSLTAEQKELLIKRLTQVASEIMEVPPEFFMAVIEELPDQNIGIGGRTIGQIKQEYQRNTGDL

SEQ ID NO: 62 - protein, 61 aa, Bacillus sp. ZYK
MPYVTIKMLEGRSNEQKKALAEKVTAVVAETTGAPLDRIHVMIEDMPKTNHAIGGKLSSEQ

SEQ ID NO: 63 - protein, 65 aa, Erwinia billingiae
MPVVNIQMFDGRDDAKPAIAKAVTDAIATHANVDPQYVYVIFNDVTTDNWAISGEIFAETLKKSG

SEQ ID NO: 64 - protein, 60 aa, Streptomyces collinus
MPIVTVQQGPRDTELKRDLVKRVTDAFVDAYGVPAESVQVWIHEVPADSWGAAGKLTADR

SEQ ID NO: 65 - protein, 61 aa, Serratia sp. M24T3
MPEIVVYAVEGRSNEQKKKLMKKITEAVVDSFEVDAERVVVSIVETKKSNKSRGGVPFDEL

SEQ ID NO: 66 - protein, 72 aa, Sphingobacterium thalpophilum
MPFVKIEITKEGVTREQKQQLIKGVTDLITNTLNKDPHLTHIVIQEVELDDWGYAGEQTSVLREKGITADKK

SEQ ID NO: 67 - protein, 60 aa, Ensifer sp. BR816
MPVIHVETFKSDDDKKADLVAEITKLFCEKMNLRPEQIHVVFDEYEPNNWATGGKLWSRM

SEQ ID NO: 68 - protein, 59 aa, Klebsiella sp. 10982
MFHIFAGKPRSRAQKQGLYRRLCARLDDEMAVSPQDVMVVIQFNQLEDWSFSGGKMAGE

SEQ ID NO: 69 - protein, 67 aa, Desulfosporosinus acidiphilus SJ4
MPFIKLEGGKIDKEKKERLISEFTRIASETLGIPEEAFVVLIKENEMDNWGTGGKVLSKILAERGNK

SEQ ID NO: 70 - protein, 67 aa, Actinokineospora enzanensis
MPVVTVDWWTGNSETARAEAVRGITDALVAAAGCPREAVTVIIRDTDPGYWGSGGTLASDSRTDRPR

SEQ ID NO: 71 - protein, 65 aa, Desulfovibrio vulgaris
MPIITVQANQLSLETRRELVHELTQSAARIMGLPPETIVVVLECRPAEDYAVGGVLLADRHREGS

SEQ ID NO: 72 - protein, 65 aa, Mycobacterium sp. JLS
MPLVEVTLVQGRAPHQLRTLITELTDAVETALGASRSTIRVVLREVPDTHWAAGDVTIAERNKGS

SEQ ID NO: 73 - protein, 61 aa, Streptomyces pristinaespiralis
MPFIEVKIYDRRLDEESERALIERLTQATVDVFGEDIRDQTWIALTAVPPRRWGLGGVPGQ

SEQ ID NO: 74 - protein, 66 aa, Bibersteinia trehalosi
MPYVNLKISTPEPSEAQLRQVVEEFTDTVARVLNKNPADVMVYIDITPPTALGFGGKTLQDIRDGK

SEQ ID NO: 75 - protein, 75 aa, Bradyrhizobium japonicum
MPFANIKIPQAALSRAQKAEIVHNLTALFAHYFGEAARTHTMVLIEEVPDGGYGRADEVFVVPDAYRASDAATSP

SEQ ID NO: 76 - protein, 74 aa, Leptospira interrogans; identical to SEQ ID NO: 12

SEQ ID NO: 77 - protein, 73 aa, delta proteobacterium MLMS-1
MPTIITQEDTMPYVNIRVAGELSKEQKAEICAGVTDHIVKVTGKPAASVLIFIDELPHENIAAGGQQLTAPGR

SEQ ID NO: 78 - protein, 63 aa, Alicyclobacillus herbarius
MPLVQLHILEGRSEAAKRQLITEVTEAVHRALGSPTESIRVLLYELPKEHWAVGGKPMSERQS

SEQ ID NO: 79 - protein, 61 aa, Amycolatopsis nigrescens
MPLINVTMFPGRTEEQKRELVRELTDAFARTCDANPDGVWVTINEVPPERWAVGGRLISDR

SEQ ID NO: 80 - protein, 64 aa, Leptolyngbya sp. Heron Island J
MPIIRVEILPGRTVEQKRELVEVLTRETARIAGCSEQSIYVVIEDVKKENWGVGGELCSDKFPD

SEQ ID NO: 81 - protein, 71 aa, Arcobacter nitrofigilis
MPYINVKMTHEDAGATKEQKEKLAKGITELFSEIFGRGASSAVILIEEVSTDNYAIGGKTVTNIREEQKKN

SEQ ID NO: 82 - protein, 72 aa, Burkholderia xenovorans
MPFANVKIPAGLIDEQQKADLVARLADTLAGFFGEGSRQTTMILIEDVADGGYYRAREIITAETLRKRPAGS

SEQ ID NO: 83 - protein, 66 aa, Calditerrivibrio nitroreducens
MPLVRIELWAGKSKDLKDAISKDVTDVLVKHLGCPPEGVTIVFDERPKSDWYTAGKSHVELHPDKK

SEQ ID NO: 84 - protein, 63 aa, Desulfotomaculum gibsoniae
MPVITVDGPKLTKEQKAKLVKSITKTASEIIKLPEQSIIVLIEERERDNVGVGGVLLSDKQPD

SEQ ID NO: 85 - protein, 66 aa, Dongia sp. URHE0060
MPFVNIRILKGHSKQRKDEISRRVTNAISEVALLPKEAVWVVFEDVESEDWYVGGDSVAEIKARQT

SEQ ID NO: 86 - protein, 71 aa, Methanosphaerula palustris
MPFVKIEHVAGEFSAEQRADLGREITDLVAKAAGVPPTLVWIVFHEIPAENWMIGRKTIPELMGETGATKK

SEQ ID NO: 87 - protein, 72 aa, Myxococcus stipitatus
MPYINVQLTGDKLATEKKLELIQRITQVFVDVLQKDPGHTFIVIQEIEPENWGVHGTSVALRRAARARGEKV

SEQ ID NO: 88 - protein, 67 aa, Terriglobus roseus
MPVIHVHMLGGASHEQKAQIAKEFTDTLVRVLGSNPQMTHIILDEQNPENWANAGELISDRRAKQKA

SEQ ID NO: 89 - protein, 67 aa, Actinomycetospora chiangmaiensis
MPLVQITLARGRTPEQLAALGEKVTAAVAEAVGAPAGNVRVVVNECEPEHWFVGGESLAQLRASGKR

SEQ ID NO: 90 - protein, 67 aa, Agrobacterium tumefaciens
MPIVKVELFPGRSAEKKAELAKAITDTLERVGGIKPEATVVLFVDIAPDNWFVAGAPLASSGFVSNN

SEQ ID NO: 91 - protein, 70 aa, Cystobacter fuscus
MPLVHIYVTPPLEDEGQRKELFRSVTEAISKSLEKPPEQTRILLHELPETDWSVAGETVDVRRRASKARK

SEQ ID NO: 92 - protein, 60 aa, Enterobacter lignolyticus
MPVLRLEMQPGRTPEQKRDFAREATRVAVETLKCKPESVDVVIVEIPRTHWAKAGLLPTE

SEQ ID NO: 93 - protein, 66 aa, Lonsdalea quercina
MPVVTVRILKEDTTAAQKAQVISGITQVLETALGKDPAKIIVMIDEVEPDNMGVGGQSITERRKSS

SEQ ID NO: 94 - protein, 71 aa, Mesorhizobium sp. LNHC232B00
MPYVKVEIAKGIASVEQKRAVIRGMTDVLVKELGRDPEYIFVVIDEVDTDNWGRKGQSLTELWRERDADRT

SEQ ID NO: 95 - protein, 64 aa, Pelosinus fermentans
MPVITIKMKKTQAEKKKELIEKLTSAAVEVTNAPANAFTVFIEEYEPDSIGIGGQTLIEKLSNK

SEQ ID NO: 96 - protein, 66 aa, Pseudomonas sp. M1
MPVIRVTWFNDKSDSVKAEVAAEFTRVLAEKTGMDSQWIHVVFENVSPCDWAVAGELFARPGEDPA

SEQ ID NO: 97 - protein, 69 aa, Sporosarcina newyorkensis
MFSMPFVHIKMIEERCTPEKKEIMIKEVTDLVAGILEQDIETVRVQIEEMRPEDWGIAGESVKKRTMKI

SEQ ID NO: 98 - protein, 62 aa, Streptomyces turgidiscabies
MPLVDVTWYEGRSHEQKEQLARAICEAFVEIGNARPDTVHVIFRDVDPTQWFKSAELLEEER

SEQ ID NO: 99 - protein, 59 aa, Thaumarchaeota archaeon SCGC AB-539-E09
MPLVQIKWFKGRDRATKAKTIELVTKAVSEACGCSTDAVTVIIEDIDKTSWGRDGKPYE

SEQ ID NO: 100 - protein, 71 aa, Actinokineospora inagensis
MPVITVQLRSGRTEQQKRELVDRLTTAFVETCGGEREGVWVALQEIPAEHWGIGGKLQSDLMGITTPPAEG

SEQ ID NO: 101 - protein, 76 aa, Advenella mimigardefordensis
MPHINIKLLAGRSEQQKQELADSISEALASTLGADDSSISVAIEDVPSGDWTEKVYKPEILGKLTSIYKRPGYKPF

SEQ ID NO: 102 - protein, 63 aa, Afifella pfennigii
MPFVVVEMWEGRTTDQKRNLVRAITDAMVTHCDCKPDHLHVVIHDVPKDSWGRAGVLGSDQKD

SEQ ID NO: 103 - protein, 62 aa, Bacillus smithii
MPFIQIHLLEGRSEEKIKDVIKNVTDCVADTLDAPLESIRVVVTEIPPTHWGTAGKTKAESK

SEQ ID NO: 104 - protein, 70 aa, Campylobacter mucosalis
MPYINIKITKENGTPTKEQKEHLARGVTELFEQILGRSGKNAVVVIDEIEPENYAVGGETISQKIQKGVR

SEQ ID NO: 105 - protein, 67 aa, Campylobacter sp. CIT045
MPYVSIKFSSPMPTKDEQDQVAKEITEILVKNLGKNPDRTVITFEEVKAESFYFAGDSVANKRSKNG

SEQ ID NO: 106 - protein, 69 aa, Desulfomicrobium baculatum
MPIISIRMAKGRSIEQKRKLVAEVTRAVSETLDLPPDIVSIQLHEFERENWATGGELHVDRFGPGFGKP

SEQ ID NO: 107 - protein, 65 aa, Desulfuromonas sp. TF
MPVVIVKTVVGSSQQQKEQLIERITDAMRDVMGKNPETTHVIIEETPEENWGLRGKTVARIRAGK

SEQ ID NO: 108 - protein, 75 aa, Erwinia toletana
MPHIIVKIVGQSEENKTLIACKLTQTLADSLGIDEQFISVSVEDIQKENWVADVYQPDILAKPASLYKKPGYNPH

SEQ ID NO: 109 - protein, 79 aa, Klebsiella pneumoniae
MVIEDTGLGLTRTRDLVVVRVYTSPRSEAQKQRFFAILQEELAEHCGLSGDDLMVSIISNQKGDWSFGRGVAQYLTGDL

SEQ ID NO: 110 - protein, 73 aa, Lactococcus lactis
MPYVHVELFEGRTVEQKAAIAKEITETISKHAGAPTSGIHVIFNDLPEGMLYQGGEMKKRNKLSFKSIKAVSK

SEQ ID NO: 111 - protein, 61 aa, Methanoculleus sp. MH98A
MPVITIRMAEGRTLEQKRILADEITAAVTKTFGVDPQVVTIFFEELKTESIARAGKLLSES

SEQ ID NO: 112 - protein, 78 aa, Mycobacterium sp. URHB0044
MPLIEVSLAQGRSEQQIRTMIHEVHEAVLRTIDTRPEYIRVIVREVPRTHWATGDVTLSEMQSRHGSDPKASGTEEES

SEQ ID NO: 113 - protein, 71 aa, Mycobacterium tusciae
MPIIEITLIEGRPPNMLREFAHAVTQAAHDLLDAPAESVRVIVHPVPPGMFFVGGKHRSALLDSTPPQDSR

SEQ ID NO: 114 - protein, 62 aa, Neisseria meningitidis 2004032
MKLRGQAAGGVGQHNVDAVQAELLARVLGKNPETTVVVIDEVDTDNWGIGGKSVSERRKEGR

SEQ ID NO: 115 - protein, 63 aa, Paenibacillus sp. PAMC 26794
MPFITVKVLEGKSVEQKRQLIERMTQVACETLDVDPSKVFIFIEDLEKDNYGKNGKLFSDLDK

SEQ ID NO: 116 - protein, 73 aa, Pelobacter seleniigenes
MPYVEVNITKGASKEQKAELVDGITELLVKVLNKNPASTHVIIKEFETDSWGISGRSVKKLRAEGKTNNISKS

SEQ ID NO: 117 - protein, 77 aa, Phaseolibacter flectens
MPHLTIKCFPRQLNQQQTEALAQALSDVLQVHLNCSEAATSIALQYVQKQDWKQQVWDRDIATQTEQLIKTPSYTPE

SEQ ID NO: 118 - protein, 77 aa, Pseudomonas agarici
MPIVNILVTREGAKRTTSEQKAAVYKGVADLLFDVMGKPREETTVIFHEHEIDDLGKGGLPLAEYLSRHAGDRQKDS

SEQ ID NO: 119 - protein, 75 aa, Rhizobium sp. AP16
MPIVTIKLLEGRSREQKTALMREVTAAVVNSLGVAPPAVRVLLEEVSPEHWSVGGISKAEPVSGGGVTPPTGDVR

SEQ ID NO: 120 - protein, 77 aa, Serratia sp. M24T3
MPHVDLKFITRNLTEAQQNAISDDLTEVLKKHFGSSDEAVSIRLNEIAKDDWKSEVYEPIIKAHLDELVKKPGYTMD

SEQ ID NO: 121 - protein, 76 aa, Serratia fonticola
MPIIKITAWPFANEKSASLLIADVTRVIHNQLGCPLDKITVYIEEISPSRWGDGGIAGDNPDFKVKSRRMCYEDGE

SEQ ID NO: 122 - protein, 74 aa, Serratia sp. S4
MPHVKIQHFPQVLSQTAKNALAEEITRAITQALQCDAGVVSIAIEPVSADDWQAQVYRPEIENKKDCLIKVPNY

SEQ ID NO: 123 - protein, 76 aa, Serratia marcescens
MPVVQVTTWKMNDETKVKKLIEEITHSVHLHCGAPLDKISVVISEISPSRWADAGVLGNDTAFPVKSRRKTYEEEA

SEQ ID NO: 124 - protein, 60 aa, Streptomyces roseoverticillatus
MPVISVDILHGRSDAAKAALARELTDAFQRTCGSKRERIYVIIREVPEEHWAIGGEMCTN

SEQ ID NO: 125 - protein, 64 aa, Thermus igniterrae
MVVLRVTLLQGRPLEKKRELAQRLTEMVARRLGEDKEEVRLILYEVPKENWAVGGVLLADREGA

SEQ ID NO: 126 - protein, 61 aa, Thiobacillus denitrificans
MPVVTIKLAGSLTRAQKQKVAEEITATLERHAGKPASYTTIAFEELPDENWAIAGKLLDEE

SEQ ID NO: 127 - protein, 59 aa, unclassified Cloacimonetes
MPIITLENAGELSKEKKQLLIKRLTEVVAEITGKPETYIYVRIDEVPRENFGIGGKSLS

SEQ ID NO: 128 - protein, 68 aa, Kingella denitrificans
MPYVNIKVTGGREAPSAEQKAELIAGVTDLLARVLNKNPETTVVVIDEVDMDNWGIGGKSVTERRKQS

SEQ ID NO: 129 - protein, 68 aa, Campylobacter jejuni; identical to SEQ ID NO: 39

SEQ ID NO: 130 - protein, 70 aa, Pseudomonas resinovorans
MPFVSVRITRDGVTREQKAQVIAEITETLERVLNKRPELTHIVIEEVDTDNWGYAGMTTTEYRRQLAEEK

SEQ ID NO: 131 - protein, 69 aa, Campylobacter coli
MPFVNIRITKENGEPTSEQKQELIAGVTDLLAKVLNKNKASTVVIIDEIDTDNYGLGGKSITQVRKEKS

SEQ ID NO: 132 - protein, 67 aa, Pseudogulbenkiania sp. MAI-1
MPYVNIKITREGATAEQKAELIRGATQLLVDVLGKNPATTVVVIDEVDTDNWGIGGESVTLRRQAGK

SEQ ID NO: 133 - protein, 68 aa, Mesorhizobium loti
MPFVNIRIVKEVIAADPAGKKADIAKKVTAAIMDATGLGNDDVWVVFEEVNARDWYVGRTDVETLRKG

SEQ ID NO: 134 - protein, 75 aa, Acinetobacter pittii
MPFINIQTIEGLLDKESKAELFKRITDLFVEIEGKGNPAFREHVWIRIDEHPPEHWQLGNLRPMKEMIELITASK

SEQ ID NO: 135 - protein, 79 aa, Rhizobium mongolense
MPIVTVQVTREGTTPDRNSVTAEEKAAIIKGVSEVLLNVLNKPLESTYVVIEEVDLDNWGWGGLPTVQYRGMRAAAAKS

SEQ ID NO: 136 - protein, 61 aa, Bacillus methanolicus
MPYVTVKMLEGRTEEQKKALVEKVTDAVSETTGAPKEKIVVFIEEMSKNHYAVAGKRLSDE

SEQ ID NO: 137 - protein, 61 aa, Streptococcus mutans
MPFVKIDLFEGRSQEQKIQLAREVTEVVSRVAKAPKEAIHVFINDMPEGTYYPHGEMKKKR

SEQ ID NO: 138 - protein, 66 aa, Budvicia aquatica
MPYVNIKITNEGATAEQKKQLIEGVTQLLVDVLNKNPATTFVVIDEVETDNWGIGGVPVTELRKAK

SEQ ID NO: 139 - protein, 65 aa, Pseudomonas sp. GM33
MPVVRVSWFEGKEHAQKAAVAAEITESIVKHTGTDANYIYVIFEDVAASDWAGAGKLFGDAPEKS

SEQ ID NO: 140 - protein, 77 aa, Sphingobium sp. C100
MPFVDIRLAGSATREQKAAIVADVTRSLVERLGKPAAAVQVVISEISTENYAAGGQLLADRAAPPSREDANAAVTPR

SEQ ID NO: 141 - protein, 61 aa, Staphylococcus aureus; identical to SEQ ID NO: 20

SEQ ID NO: 142 - protein, 75 aa, Escherichia coli
MPHIDIKCFPRELDEQQKAALAADITDVIIRHLNSKDSSISISLQQIQPESWQAIWDAEIPPQMEALIKKPGYSM

SEQ ID NO: 143 - protein, 70 aa, Mesorhizobium loti
MPFANIKLPQAALSKAQKEDLIHRTTAMFVDFFGEGVRPTTMVLVEEVADGGYGRADEVFVIPEAYRAKE

SEQ ID NO: 144 - protein, 70 aa, Acinetobacter sp. NIPH 817
MPYILIQITKENVSIQQKQQLIQEATELVVRVLDKDPATTFVVIDEVETDNWGVAGETVSAIRKRQSLST

SEQ ID NO: 145 - protein, 68 aa, Pseudomonas putida
MPYVHIRVTDEGVSAEHKRQLIEQTTCMLERVLGKPPASTFVVIEEVPTDNWGVGGETVTAVRARERG

SEQ ID NO: 146 - protein, 70 aa, Rhizobium sp. CF080
MPFVNIKTPEGALSKAQKEEIVHRTTEMLVEYMSEAARPHTMVLIEEVKDGGYGRADEIFMIPDAYRARE

SEQ ID NO: 147 - protein, 71 aa, Calothrix sp. PCC 7507
MPFVTVQIAKGHSIEKKRQLVQAVTDALVATLGTKPEWITVYIDEFERENWAVGGQLHADKHSGRHKEKGV

SEQ ID NO: 148 - protein, 78 aa, Bradyrhizobium japonicum
MPIVTIQVTREGTTPGAASLTAEEKAALIKGSSQLLLDVLGKPLESTFVVIEEVDTDNWGWGGLPVQEFRRRSAAQTG

SEQ ID NO: 149 - protein, 63 aa, Azoarcus toluclasticus
MPFAQIYLVEGRSEEQKRAVIEKVTQALVEAVGAPKENVRVWIQDVPKTNWGIAGQTAKDLGR

SEQ ID NO: 150 - protein, 75 aa, Citrobacter sp. 30 2
MPHVDIKCFPRALTDEQKTALAADITEVIIRHLQSKDSSVSVALNEVEPEAWQAVWDTEIAPQMARLIKKPGYSM

SEQ ID NO: 151 - protein, 76 aa, Enterobacter cloacae
MPHVDIKCFPRDLNDEQKNALAADIAEVIIRHFNSKDSSVSVALNQVNPEDWKAQVWDTEIGPKLDELIKKPGYSM

SEQ ID NO: 152 - protein, 78 aa, Burkholderia sp. YI23
MPIVTIQVTREGNTPGTNAVTADEKAQLIKGVSELLRDVLNKPLDSTFVVIQEVELENWGWGGLPVPEYRRQKQAKDS

SEQ ID NO: 153 - protein, 78 aa, Mesorhizobium opportunistum WSM2075
MPIVTVQLTREGTEPGVNHVTAEQKAAIYKGMSEVLLDVLGKPLEWTWVVFEEVEMENWGWGGMPVAEYRKLLAAKGV

SEQ ID NO: 154 - protein, 61 aa, Persephonella sp. IF05-L8
MPYVNVKVAGELTREQKQKIVEGITKVLEEVANKPPSATYVVIEEVDRDNWGKGGKLLSDK

SEQ ID NO: 155 - protein, 72 aa, Pseudomonas syringae group genomosp. 3
MPLVTIKGIEGVFTDEQKAEVIRKVTDAMVSVEGENMRALTWVIFEEVKSGDWGIAGNPVTAEDVLKLQAGQ

SEQ ID NO: 156 - protein, 73 aa, Leisingera methylohalidivorans
MPYVNIKVTREGGPDGTGPSAEQKAQLIAGVTELLQDVLGKNPATTFVVINEVPLEDWGVGGLPVQEYRKQAQ

SEQ ID NO: 157 - protein, 71 aa, Pseudomonas aeruginosa; identical to SEQ ID NO: 18

SEQ ID NO: 158 - protein, 77 aa, Bradyrhizobium diazoefficiens USDA 110
MPFANIKIPQAALSRTQKAEIVHRLTALFVDYFGEAARPHTMVLIEEVPDGGYGRADEVFVVPDAWRARDAVTPASG

SEQ ID NO: 159 - protein, 67 aa, Campylobacter concisus; identical to SEQ ID NO: 54

SEQ ID NO: 160 - protein, 62 aa, Nitrosospira multiformis
MPYVNIRIAGSLSREQKKQIAQEITDTLERIAHKPKSYTYIAFDEMPHENWAIAGTLLGEED

SEQ ID NO: 161 - protein, 60 aa, Streptococcus pseudopneumoniae
MPFVRIDLFEGRTLEQKKALAKEVTEAVVRNTGAPQSSVHVIINDMPEGTYFPQGEMRTK

SEQ ID NO: 162 - protein, 71 aa, Streptomyces violaceusniger
MPFANFKVPAGSLDEKQKQEIVTRTTELYVEIYGERARANTLVVVEEVTDGGWGIGGNVLTLAMLQQPPGH

SEQ ID NO: 163 - protein, 72 aa, Serratia sp. S4
MPYVNIKVTDEGVTAEQKRQLIEGVTELLERVLNKPRYATSVVIDEVATDNWGVGGESVTALRNKERNSGQR

SEQ ID NO: 164 - protein, 67 aa, Asticcacaulis sp. AC460
MPYVNIRITREGATPEQKAALIKGATDLLVEVLGKNPATTFVVIDEVELDNWGVAGETVRELRKKGQ

SEQ ID NO: 165 - protein, 68 aa, Azospirillum lipoferum
MPYVNIKITREGATPEQKAELIRGVTDLLVDVLDKTPATTFVVIDEVDMEDWGVGGLPVAEYRKTLGT

SEQ ID NO: 166 - protein, 72 aa, Chitinophaga pinensis
MPYVKIEVTREGVTREQKQALIKGVTDLITDVLNKDPQLTHVVIQEIELDDWGFAGEQVSVLREKGITADKK

SEQ ID NO: 167 - protein, 78 aa, Enterobacter cloacae
MPFVNVQTIKGIMNAEQKSELLRRMTDLMVEIEGKGDPEFRRSVWIRIDEHDPEQWSLGGLQPTAEMIAKKFASTQAS

SEQ ID NO: 168 - protein, 59 aa, Leptospira inadai
MPYVNVKVAGPLTKEQKQTIVKEFTETLRKVAAKPPESTYIVIDEVSREDWAAGGKLLE

SEQ ID NO: 169 - protein, 61 aa, Nocardia seriolae N-2927
MPIVTVQQGPRTVELKRDLVRRITDAFVDAYQIPAEAVQVWIHETPADSWGTAGRLTADGK

SEQ ID NO: 170 - protein, 65 aa, Pseudoalteromonas haloplanktis
MPVVNIQMFEGRDHAKQAIAKEVTASIAKHADVDPQYVYVIFNDVKTEDWAISGEVFAETLKKSS

SEQ ID NO: 171 - protein, 71 aa, Anabaena variabilis
MPFVTVKIARGHSIEKKRHLVEAITNALVTALDTKPEWITVHIDEFERENWAVNGILHCDRHRGRHDETGR

SEQ ID NO: 172 - protein, 72 aa, Burkholderia cepacia complex
MPLINVKVIEGVFSAADKEKIISTLTDAMVSIEGESMRPVTWVIVEEVKSGDWGIAGNALTTERVKALAAGQ

SEQ ID NO: 173 - protein, 68 aa, Helicobacter pylori
MPFINIKLVPENGGPTNEQKQQLIEGVSDLMVKVLNKNKASVVVIIDEVDPNNYGLGGESVHHLRQKN

SEQ ID NO: 174 - protein, 72 aa, Alcanivorax dieselolei
MPFANYKFPEGILDHAKKEEIIHRTTAMFVEYFGEGVRPYSMVLVEEVADGGWGRADETLTLEKMGLPPKSR

SEQ ID NO: 175 - protein, 70 aa, Arcobacter butzleri
MPVINIKMTKEDGGATKEQKEKLSKGITELFAQTFNGRGASTAVVIIEEVSTDNYSIGGKTISQIREEAK

SEQ ID NO: 176 - protein, 64 aa, Brevibacillus brevis
MPYINLQITKGATRDQKAQIVKEFTETLVNVLGKKPEHTHIVIEEIDDENWGFSGILTDDFRKQ

SEQ ID NO: 177 - protein, 78 aa, Burkholderia terrae
MPIVTIQVTREGTKPGADCVTAEEKARLIKGVSQVLLDVLNKPLESTFVVIEEVPTDNWGWGGLPALEYRKLKAAKSA

SEQ ID NO: 178 - protein, 59 aa, Gordonia bronchialis
MPLIQITQTPGLSDRVKRETIAAVTEAYAMATGKNPDSVWVTINEIPRQQWGVGGEPLG

SEQ ID NO: 179 - protein, 61 aa, Listeria innocua
MPFVTIQFLEGRSDDQKKALVSEVTDVVSKNLKAPKENIHVILEEMKKTDYGVGGVRKSDI

SEQ ID NO: 180 - protein, 69 aa, Bacteroides vulgatus
MPYISVESGALTSEQKKELIERLTATAAEITHIPEQFFTVTIKELPDENFGIGGKSIDKIKRNYIGKEH

SEQ ID NO: 181 - protein, 71 aa, Campylobacter fetus
MPYVNIKVTKENGEPTVAQKEELIKGVTELLHTVLGRNRASTVVIIDEIETENYGLGGESIKIKRKREEKR

SEQ ID NO: 182 - protein, 66 aa, Desulfovibrio magneticus
MPFVNIKITRDGATAEQKATVIAGVTNLLRDVMGKNPQTTVVIIEEVDTDNWGIGGETVTARRARG

SEQ ID NO: 183 - protein, 73 aa, Janthinobacterium sp. Marseille
MPYVNIRVTREGVTAEQKLQLIEGATDLLSRVLNKNPATTFVIIDEVDTDNWGVARENVTTLRKREAEKKAAS

SEQ ID NO: 184 - protein, 60 aa, Ensifer sp. BR816; identical to SEQ ID NO: 67

SEQ ID NO: 185 - protein, 60 aa, Methylophilaceae bacterium 11
MPYVNIKLAGAVTREQKAEIAKEITETLARVAHKPANYTYITFEEVEYEDWAIGGKLLDE

SEQ ID NO: 186 - protein, 76 aa, Sphingomonas melonis
MPFVSIRLAGSATKEQKAGVVADVTASLVTRLGKNPAAVQIVIEEVSTENYGAGGQLIADRDAPQVREDADAVPSQ

SEQ ID NO: 187 - protein, 72 aa, Synechococcus sp. PCC 7336
MPYVNIQITKGATREQKAELVEDVTDSLVRVLGKKPEHIHIVIQEINDEDWGFSGLLTDEWKKQQSQLPASE

SEQ ID NO: 188 - protein, 71 aa, Azotobacter vinelandii
MPYVNIKITREGATAEQKRRLIEGATRLLVEVLGKNPATTVVVIDEVDTDNWGIGGIPVTERRRSEKQQPN

SEQ ID NO: 189 - protein, 66 aa, Azovibrio restrictus
MPYVNIQITKGATRAQKAELVREVTASLVRVLGKRPEHTHVVIQEIEEEDWGYAGLLTDDWRRQQG

SEQ ID NO: 190 - protein, 70 aa, Campylobacter rectus
MPYVNIKITKEKEVVSAEQKRALIAGVTKLLGDTLKRDASRTVVVIEEVSTDDYGFGGESITELRSKQGK

SEQ ID NO: 191 - protein, 76 aa, Cronobacter sakazakii
MPHIDVKFFPRDLSDAQQQALADELTEVIVKHLQSKESSVSVALNEVQPEQWKSEVWDTEIAPQLDTLARKPGYEM

SEQ ID NO: 192 - protein, 70 aa, Helicobacter pylori
MPFVNVRITKEQGCPTTEQKKELIVGITELLSKALNKNKASTVVIIDEIDTDNYGLGGKSITEARKQTES

SEQ ID NO: 193 - protein, 59 aa, Klebsiella sp. 10982; identical to SEQ ID NO: 68

SEQ ID NO: 194 - protein, 63 aa, Pseudomonas fluorescens group
MPYINLQITKGATRQQKAQLVKEFTESLVRVLGKKPEHTHIVIQEIDGSDWGFAGELSDDDQA

SEQ ID NO: 195 - protein, 70 aa, Salinispora arenicola; identical to SEQ ID NO: 60

SEQ ID NO: 196 - protein, 68 aa, Sulfurospirillum multivorans
MPYVTIKITKEGATKEQKAELIEGVTNLLVSVLGKNPATTVVTIDEVDMDNWGIGGQQVSELRKKPKP

SEQ ID NO: 197 - protein, 67 aa, Bacillus sp. 37MA
MPYVNIKITNENVTPEKKALLVKGATQLLVDVLGKNPNTTVVVIDEVNTDNWGIAGETITVRRKNGQ

SEQ ID NO: 198 - protein, 70 aa, Bradyrhizobium oligotrophicum
MPFANIKVPQAALTTAQKEEIVHRVTAMFVDYFSEAARPHAMVLIEEVPDGGYGRADEVFVIPDAYRATS

SEQ ID NO: 199 - protein, 70 aa, Celeribacter baekdonensis
MPFANYKLPQDSLTTAQKEDLIHKTTDLLVGLFGEVVRPNTMVLIEEVKDGGYGRADEVFVLPDEYRAKG

SEQ ID NO: 200 - protein, 65 aa, Desulfovibrio vulgaris; identical to SEQ ID NO: 71

SEQ ID NO: 201 - protein, 74 aa, Helicobacter felis
MPYINIRISKEHGGATTEQKRALISGVTDLVVDILGKKREATIVIIDEIHTDNCGLGGETITRLRHTPPEQLKD

SEQ ID NO: 202 - protein, 68 aa, Moritella marina
MPYINIKITDEQVTKEQKAQLIAGATQLLVDVLGKNPKTTVVVIDEVNTDNWGIGGEVVTELRQKAKT

SEQ ID NO: 203 - protein, 75 aa, Acinetobacter venetianus
MPYILIQITKENVSQQQKQQLIQEATELVVRVLDKDPATTFVLIDEVDTDNWGVAGESVTTLRQKQAKSTVHQVE

SEQ ID NO: 204 - protein, 72 aa, Bacillus clausii KSM-K16
MRQMPYVNIKVTKENGGLTKEEKLTLIKGVTNLLAETLNKNPASTVVVIDEIDMDNYGLGAEQISDRRKAGK

SEQ ID NO: 205 - protein, 66 aa, Bibersteinia trehalosi; identical to SEQ ID NO: 74

SEQ ID NO: 206 - protein, 76 aa, Campylobacter curvus
MPFVNIRITKENGEPTREQKERLIDGVTQLLADVLGKNKDSTVVIIDEIDTDNYGLGGKTITNVRKLAKEQKELKC

SEQ ID NO: 207 - protein, 67 aa, Campylobacter rectus
MPFINVKMAGPEPTKEQKQKLVEEMTDTMVRVLGKSRERVQIYIETYDASSIGSGGKTLEELRKEQK

SEQ ID NO: 208 - protein, 64 aa, Cupriavidus sp. UYPR2.512
MPVVRVSWFEGKDAKAKKAVAAEITDSIVKNTGTDPKYIYVIFEDVKPSNWAVAGRLYGEDPKP

SEQ ID NO: 209 - protein, 67 aa, Leptothrix cholodnii
MPVVRVSWFEGKDHAQKEKVAAEITDSIVRHTATDPNYIYVIFEDVKASDWAGAGKLFGKSPEAGEG

SEQ ID NO: 210 - protein, 67 aa, Marichromatium purpuratum 984
MPYVNIKITREGASAEQKAELISGVTELLARVLDKNPQTTVVVIDEVETDNWGIGGETVTVRRASGR

SEQ ID NO: 211 - protein, 76 aa, Methylobacterium nodulans
MPIVTIQVTREGSAPGRSSVTAEEKASLIAGVSQLLLDVLHKPLDSTFVVIEEVELENWGWGGLPVAEFRKQRRPR

SEQ ID NO: 212 - protein, 76 aa, Pectobacterium carotovorum
MPHIDVKHFPRNLSEEEKKAVAEDLAAVLKKHFGSSDESLSVALNEIQPERWKDDVYDPFIKPQLDTLPKKPGYSY

SEQ ID NO: 213 - protein, 68 aa, Phascolarctobacterium sp. CAG207
MPYVNIKITREGNVTPEQKQALIEGATNLLHDVLGKNKKTTVVVIDEVDTDNWGIGGISVTEIRKRNN

SEQ ID NO: 214 - protein, 76 aa, Streptomyces sp. PVA 94-07
MPFANFKVPENTLTPKQKEEIVTRTTELYVELYGERARDNTMVLVEEVADGGWGISGNVLTLAMLGEAVPTDTSGA

SEQ ID NO: 215 - protein, 73 aa, Variovorax paradoxus
MPFVNLKITRDGVTREQKTQVIAEFTETLERVLKKPPEWTHIVIEEIDTDDWGFAGLTTTEYRKRLADRAKPA

SEQ ID NO: 216 - protein, 71 aa, Caulobacter vibrioides
MPYVNIQVTREGVTREQKARLIDGVTTLLAEVLGKDPNTTFVVIEEVALENWGVGGLPVEAYRARKRPAAT

SEQ ID NO: 217 - protein, 72 aa, Laribacter hongkongensis
MPFINIRLTREGATQEQKARLIEGVTDLVTTVLGKNRATTFVIIDEVETDNWGIGGEPVTQIRARQSQTSQA

SEQ ID NO: 218 - protein, 74 aa, Leptospira interrogans; identical to SEQ ID NO: 12

SEQ ID NO: 219 - protein, 71 aa, Lyngbya aestuarii
MPFVTIQIGEGHSIEKKRELAKAVTDTLVETLGTKREWVTIHIDEFNRDNWAVGGELHSDKHPGRHADKSV

SEQ ID NO: 220 - protein, 59 aa, Paracoccus denitrificans
MPVVRVSWFEGKAAEEKQKVAQDITDSIVRNTGTDASYIYVIFEDVKTSDWAGGGKLYG

SEQ ID NO: 221 - protein, 68 aa, Pectobacterium carotovorum
MPYVNIKVTGGKEAPTAEQKSELIREVTQLLARVLNKNPATTVVIIDEVDMDNWGVGGETTTQRRKQQ

SEQ ID NO: 222 - protein, 71 aa, Pseudomonas denitrificans
MPYVNIQVTDEGVTAEERRQLIEGVTELLERVLRKPPASTFVVIQEIPTDSWGVGGETVKALRARAKQPGD

SEQ ID NO: 223 - protein, 71 aa, Streptomyces prunicolor
MPFANFKVPVGALTAEQKEQIITRTTDLYAEIFGDGARATTLVLVEEVADAGWGIGGGVLTLAKIQGTPQE

SEQ ID NO: 224 - protein, 64 aa, Thermoanaerobacter ethanolicus JW 200
MPFITIEGPDNLSKETKAKLISELTKAASQVLNIPINAFSVIIKENNPDNIGVGGTPLSELHKK

SEQ ID NO: 225 - protein, 61 aa, Thiobacillus thioparus
MPVVTIKLAGSLTREQKQKIAEEITATLERHAGKPASYTYIAFEELPDENWAIAGKLLDED

SEQ ID NO: 226 - protein, 70 aa, Yersinia pestis
MELNMPYVNIKITREGATAEQKKQLIAGVTQLLVDTLGKNPATTVVVIDEVETDNWGIGGRSVTDLRQSS

SEQ ID NO: 227 - protein, 71 aa, Acinetobacter sp. NIPH 1859
MPYVNIKVTDEGVTPEHKQQLIEGVTELLYQVLGKPHATTFVVIDEVNTDNWGVAGTTVTALRQASSSEPQ

SEQ ID NO: 228 - protein, 60 aa, Aeromonas salmonicida subsp. masoucida NBRC 13784
MPVVRVTWFEGKETGTKQVVAAEITESIVRNTGTDPKYIYVIFEDVAPSDWAGEGKLFGD

SEQ ID NO: 229 - protein, 61 aa, Amycolatopsis alba
MPMINVSMFPGRTAEQKQALVREVTDAFVRTCGGNPDGVWVTINEIPAEHWASGGTLFSDR

SEQ ID NO: 230 - protein, 70 aa, Burkholderiales bacterium 1 1 47
MPFVRICVTGDEERPTSEQKKELIAGVTKLVGDVLGKNTSNMVVIIDEIPMENYGIGGKTVRERRKEKQS

SEQ ID NO: 231 - protein, 67 aa, Campylobacter curvus
MPFINVKMAGPEPSKEQKAKLVEEMTEVMVRVLGKQKERVMIFVETYEPDSIGLGGKTIEQIKKDSK

SEQ ID NO: 232 - protein, 68 aa, Campylobacter lari
MPIINIKLAKPEFSKAQKEELIADITELLHTKYNKPKQNIVIMLEDIEPYNIAFGGESVEKLRMKGKK

SEQ ID NO: 233 - protein, 60 aa, Campylobacter ureolyticus
MPFVKICVTKENNEPTKEQKELLINEVLGKNKSSIVVIIEEIDTDNYGLGGKSITNLRKK

SEQ ID NO: 234 - protein, 68 aa, Campylobacter upsaliensis
MPFVNIKITKENETPTKEQKEALIQGVTELIAKILNKNKASTVVIIDEINTDNYGLGGESITQVRKRV

SEQ ID NO: 235 - protein, 69 aa, Cedecea davisae
MPYVNIRVTKEGVSAEQKQALIEGTTDLLMRVLNKDPATTFVIIDEVETDNWGVNRESVTSRRRREARG

SEQ ID NO: 236 - protein, 71 aa, Chamaesiphon minutus PCC 6605
MPYINIKITREGATAKQKAELIKGVTELLVRVLNKTPATTFVVIDEVATEDWGIAGVSVKELRQGIQPQSQ

SEQ ID NO: 237 - protein, 61 aa, Desulfobulbus propionicus
MPYVSIRVAGKLTKEQKQKIAKGVTDVISEAAGKPKESILLFIDEESRDSIAKGGVLIEDM

SEQ ID NO: 238 - protein, 69 aa, Dialister succinatiphilus
MPYVNIKITREGQVTPEQKARLIKGVTDLLADVLHKNPATTVVVIDEVPLENWGLGGDPILVAREKRKK

SEQ ID NO: 239 - protein, 78 aa, Dyella ginsengisoli
MPYINIQVTREGTAPGRSAMTEEEKAALIAGASQLMLDVLDKPLESTFVVIDEVPTENWGWGGLPALEFRRRRAAGKP

SEQ ID NO: 240 - protein, 72 aa, filamentous cyanobacterium ESFC-1
MPFVTIQIAQGHSVAQKRDLAKAVTDTLVAQLGTKPEWVTIHIDEFDRENWAVGGELHIDKPSRKHQKESLK

SEQ ID NO: 241 - protein, 71 aa, Helicobacter bizzozeronii
MPFINIKITQEEGQPTKEQKQALIAEVTQVVAKVLKRDRVASTTVIIEEISTDNYGLGGKSVTQIRGSGQL

SEQ ID NO: 242 - protein, 60 aa, Herbaspirillum sp. CF444
MPTFNIQLFEGRSVEQKRELVKAITDVTCKTLQCSPESVDIIIQDVKKENWATAGKLWSD

SEQ ID NO: 243 - protein, 61 aa, Herminiimonas arsenicoxydans
MPTINVQLFEGRTIEQKRAFVKAVTEATVATLGSSAESVDIIIEDVKRENWATGGKLWSDV

SEQ ID NO: 244 - protein, 71 aa, Labrenzia aggregata
MPYVNIQVTREGVTAEQKARLIKGATDLLVDVLDKNPATTFVVIEEVDLENWGVGGLPVEDYRARQLAKQS

SEQ ID NO: 245 - protein, 65 aa, Mesorhizobium metallidurans
MPFANFKVPERALSAGQKEDLVHKTTALFVEMFGEGVRPYTMVLIEEVADGGYGRADVVFKIPGG

SEQ ID NO: 246 - protein, 61 aa, Methylobacillus flagellatus
MPYVDIKVAGTLTREQKAKIAEEITDTLERIAKKPKSYTYITFHEVAGENWAVAGELLDSD

SEQ ID NO: 247 - protein, 73 aa, Methylomonas methanica
MPLVQIKGISGYLSIAQKQEIIRKVTDAVVSVEGEGLRPVTWVLIEDVASGEWGVGGEAVTTEALRNMSLATG

SEQ ID NO: 248 - protein, 60 aa, Methylovorus glucosetrophus SIP3-4
MPYINIKHAGPLSREQKAKIAEEVTKTMERVANKPASYTYITFDEVEYEDWAIAGKLLDE

SEQ ID NO: 249 - protein, 67 aa, Nocardia brasiliensis
MPYVNIKVTDEGVTREQKSRLIAGVTELLRDVLGKDPATTFVVIDEVALANWGVGGVDVEEYRRSRF

SEQ ID NO: 250 - protein, 61 aa, Pantoea sp. GM01
MPEIVVYAAEGRTVEQKKKLLRAVTAAVADSFEIDANSVTVSIIETPKHHKSKGGVPFDER

SEQ ID NO: 251 - protein, 71 aa, Pleurocapsa minor
MPFVTIQIAKGHSVELKRKLAQAVTDTLVSTLGTKPEWVTIHIDEFDPENWAVGGELHIDRHSDKHAQKKS

SEQ ID NO: 252 - protein, 63 aa, Pseudomonas putida; identical to SEQ ID NO: 17

SEQ ID NO: 253 - protein, 74 aa, Rhodomicrobium vannielii
MPIITVRIAKGRPTETKRALAAALTRSAAETLDVPAEWVTVLIEEYDRENWATGGELHADKFGKGFGRKGSDEA

SEQ ID NO: 254 - protein, 71 aa, Rivularia sp. PCC 7116
MPFITIQIAKGHSIEKKRELAKAITDAMVSTMGTKPEWVTIHIDEFERNNWSVGGTLHSDKHKGRHKEKGV

SEQ ID NO: 255 - protein, 68 aa, Streptomyces ghanaensis ATCC 14672
MPVITVNWWQGNDRKARKDLVTGITDVVTRVSGCPDEAVTVIVRDVAPDHWSTGGRLADETRPPRGRR

SEQ ID NO: 256 - protein, 67 aa, Symbiobacterium thermophilum
MPYVNIKITREGATPEQKKQLIAGATRLLQEVLGKNPQTTFVIIEEVDTDNWGIAGETVTERRKKGM

SEQ ID NO: 257 - protein, 66 aa, Tolumonas auensis
MPVVNIQMFEGRSDESKQKIAKDVTDSIAKHTGVDPQYVYVIFNDVELKNWAISGEVYSETLKKNS

SEQ ID NO: 258 - protein, 72 aa, Achromobacter xylosoxidans
MPYINIQITREGATREQKAELIKGATDLVVRVLGKDPQATFVVIEEVDTDSWGVAGETVTALRQRQRAAQAT

SEQ ID NO: 259 - protein, 78 aa, Achromobacter xylosoxidans
MPFVSIRITRDGVTAEQKAQVIREVTETLERVLHKPPEWTHVVIEEIDTDNWGFGGVTTTEMRAGRAPTPGAKPQGRG

SEQ ID NO: 260 - protein, 75 aa, Acidovorax sp. NO-1
MPYINIKVTREGVTGAQKAALIQGATDLLVRVLNKEPATTFVVIDEVETDNWGVAGETVTVRRQRARAAADGSAP

SEQ ID NO: 261 - protein, 74 aa, Acidovorax radicis
MPLVNIRLARRDLPTTAAQKAALIAGVTQLMRQVLDKRPETVTVIIDEVDPENWGQGGEPVTVIRQRNKGPAQA

SEQ ID NO: 262 - protein, 67 aa, Actinoplanes sp. SE50/110
MPYINVKITREGVTREQKRRIIGEMTQVLVDVLGKDPRTTFVVIDEVELDDWGIGGLPVPEFRRNGR

SEQ ID NO: 263 - protein, 73 aa, Actinoplanes friuliensis
MKENAMPFANLKVPAGLLTPEQKETLVHKVTDLYAETFGDDVRDNTMVLVDEVVDGGWGIRGEVLTLAKLSGN

SEQ ID NO: 264 - protein, 68 aa, Aggregatibacter segnis
MPYVNIKVTGGAEAPTAKQKEELIEGVTHLLQKVLNKNPETTVVIIDEVDMDNWGIRGKTVTSRRSMK

SEQ ID NO: 265 - protein, 64 aa, Aquimarina macrocephali
MPYINIKVTDEQVTTDQKRQLIEGTTQLVVDILNKNPKTTHVVIEEVPIENWGVNGKQYLGTKK

SEQ ID NO: 266 - protein, 71 aa, Arcobacter nitrofigilis; identical to SEQ ID NO: 81

SEQ ID NO: 267 - protein, 69 aa, Azospira oryzae
MPYVNIKITREGATAEQKATLIRGVTLLLRDILGKNPETTVVTIDEVDTDNWGIGGDSVTVRRARERQG

SEQ ID NO: 268 - protein, 61 aa, Bacillus sp. NSP9.1
MPYVTVKMLEGRTDEQKRNLVKKVTEAVSETTGAPAEKIAVMIEEMKKEHYGVAGKRISDM

SEQ ID NO: 269 - protein, 69 aa, Bartonella grahamii
MPYINIKITNEGVTSEQKRQLIEGATQLLVNILNKNPKTTVVVIDEVETDNWGINGVPVTELRKDQNKE

SEQ ID NO: 270 - protein, 66 aa, Bdellovibrio bacteriovorus
MPYVNVQITKGATKEQKAQIIADITQSLVKVLGKKPEHTHIVIQEIADEDWGFEGLPTDEWKKKQK

SEQ ID NO: 271 - protein, 74 aa, Bordetella petrii
MPYVNIKVTREGTAPGASATTPEQKKALIKGVSDLLFEVMGKPRNSTFVVIEEVDMDSWGVGGVTVAEYRKHGD

SEQ ID NO: 272 - protein, 80 aa, Bradyrhizobium sp. DFCI-1
MPIVTIQVTREGTTPGASSTTPEQKAALIKGSSELLRDVLGKPMESTFVVIEEVDTDNWGWGGLPALEYRKKRAAAAASR

SEQ ID NO: 273 - protein, 62 aa, Burkholderia ambifaria
MPTFNIQLFEGRTVEQKRAFVEAITRATCETLGCDPGSVDIILADVKKENWATAGKLWSDEL

SEQ ID NO: 274 - protein, 72 aa, Burkholderia xenovorans; identical to SEQ ID NO: 82

SEQ ID NO: 275 - protein, 78 aa, Burkholderia phytofirmans
MPIVTIQVTREGSKPGTNAVTADEKARLIAGVSQVLLDVLNKPLAATFVVIEEVDMENWGWGGLPVEAYRKQLAQKPG

SEQ ID NO: 276 - protein, 66 aa, Calditerrivibrio nitroreducens; identical to SEQ ID NO: 83

SEQ ID NO: 277 - protein, 72 aa, Chryseobacterium gleum
MPFIKVDVLREDLNRETKQQLIRKISEAVTSVLNKDPHLTHIVINEIEDDNWGYAGEQVSVLKEQGFSTVKK

SEQ ID NO: 278 - protein, 70 aa, Collimonas fungivorans
MPYVNIRVTREGVTAEQKQQLIAGVTELLERVLNKKPATTFVVIDEVDTDNWGAGGESVTVIRKREAAAR

SEQ ID NO: 279 - protein, 68 aa, Colwellia psychrerythraea
MPYINVKITEEGVTNEQKQTIIKGCTQLMVEVLNKDPSTTFVVIDEVSTDNWGIGFEQVTELRKQQKR

SEQ ID NO: 280 - protein, 67 aa, Cupriavidus pinatubonensis
MPYVNIRITREGTTAAQKAQLIRGATQLLADVLGKDPRSTVVVIDEVDPDNWGFAGETTTERRKAGR

SEQ ID NO: 281 - protein, 66 aa, Cytophaga hutchinsonii
MPYVNIKVTDTGVTSEQKAALIKGVTDLLQQVLNKNPLTTVVVIDEVSTDNWGIGGEQVTSRTKKS

SEQ ID NO: 282 - protein, 66 aa, Dasania marina
MPYVNIKVTDENVTKEQKQALIAGTTQLLVDVLNKNPATTFVVIDEVNTDNWGIGYDQVTALRKKK

SEQ ID NO: 283 - protein, 66 aa, Desulfobacterium autotrophicum
MPYVNIKITDENVTKDQKLKLIQGVTQLLVDVLGKNPATTVVVIDEVHTDNWGIGGKCVTELRTRK

SEQ ID NO: 284 - protein, 67 aa, Desulfocapsa sulfexigens
MPFVNIKITNEDVTPEKKAELIKGATELLKNVLGKNPATTVVVIEEIDTDNWGIGGETVTQRRKNGK

SEQ ID NO: 285 - protein, 62 aa, Desulfocapsa sulfexigens
MPYVNIRVAGTLSKDQKSNICKGVTEVIAREAGKPPEAILIFIDEVPHENIAKAGNLLTPPK

SEQ ID NO: 286 - protein, 67 aa, Desulfosporosinus acidiphilus SJ4; identical to SEQ ID NO: 69

SEQ ID NO: 287 - protein, 63 aa, Desulfotomaculum gibsoniae; identical to SEQ ID NO: 84

SEQ ID NO: 288 - protein, 67 aa, Desulfovibrio vulgaris
MPYVNIRITREGATSEQKAALIQGVTQLLVDILGKNPATTVVTIDEVDTDNWGVGGETITARRKRGV

SEQ ID NO: 289 - protein, 63 aa, Desulfurivibrio alkaliphilus
MPYVNIRVAGTLSKEQKAEISAGVTEVITRVANKPANSVLIFIDELPHENIASAGQLLQPPAK

SEQ ID NO: 290 - protein, 76 aa, Dickeya solani
MPHVAIKYFPRNLPETAKQDLANEISELLKKYLNAEDKSISVALTEVAPESWKEAVYDIEIGPELDNLTKKPGYTL

SEQ ID NO: 291 - protein, 59 aa, Dietzia alimentaria
MPFIQINQLDGMDAAGKARVIEAVTAAYAESTGKDPAKVWVHVNDMARDSFGIGGKAVG

SEQ ID NO: 292 - protein, 66 aa, Enterobacteriaceae bacterium 9 2 54FAA
MPFVNIKITREGATAEQKEALIAGVTQLLVDTMGKNPATTVVIIEEVETDNWGIGGKSVTELRKKK

SEQ ID NO: 293 - protein, 79 aa, Frankia alni
MEVTDMPFVDVKVIEGVFTPQQKQEMITKLTEVMVGIEGEAMRSVTHVVVEEVRSGDWGIGGRCMTAEDVQKLQVSGST

SEQ ID NO: 294 - protein, 62 aa, Gallionella capsiferriformans
MPYVNIRIAGTLSREQKAAIASEVTDTLERIAHKPASYTYITFDEVAEENWAVAGKLLDSDA

SEQ ID NO: 295 - protein, 67 aa, Gallionella capsiferriformans
MPYVNVQITKGATKSQKAEIVKGITNSLVRVLGKKPEHTHIVIQEIEEQDWGFAGLLTDEWKKQQSA

SEQ ID NO: 296 - protein, 67 aa, Hahella chejuensis
MPVVTIAQSPGRSLEQKRELVKKITDAFVTSYGVKPESVTIFLQDYDDSNWGKAGLLHVDAQAGKDG

SEQ ID NO: 297 - protein, 63 aa, Haliea salexigens
MPVAQINILEGRTDEQKEMLIHEVTDAIVRSLGAPVESVRVILNEMPKQHFGIGGQSAKKLGR

SEQ ID NO: 298 - protein, 64 aa, Halomonas campaniensis
MPIVTIQQFPRDLAQKRELAKRITDAFCDVYGTDPVSVQVFFQEVTRENWSKGGQMGVDRDTAQ

SEQ ID NO: 299 - protein, 61 aa, Halothiobacillus neapolitanus
MPVVTIKLAGNLTREQKKQIAAEVSDTLERVAQKPKRATYITFEELPREDWAVGGQLLDEK

SEQ ID NO: 300 - protein, 68 aa, Helicobacter felis
MPFVNIKLTRESTPITPEQKKALIEGATDLLVKVLNKNRATTIVIIEELDMDNWGIGGNSVTEIRKKS

SEQ ID NO: 301 - protein, 69 aa, Hirschia baltica
MPYVNIKITREGNVTAAQKASLIKGTTELLRDVLGKDPNTTMVVIDEVDLDNWGIGALPALEFRAQRQK

SEQ ID NO: 302 - protein, 68 aa, Hydrogenophaga intermedia
MPYVNIQVTRENVSAPQKAELIAGVTELLQRVLGKDPALTHVVIDEIDPDNWGVGGLPALQYRQRGAR

SEQ ID NO: 303 - protein, 70 aa, Janthinobacterium sp. HH01
MPYVNIRVTKEGVTAAQKLELIEGVTDLLTRVLGRDPATTFVIIDEVETDNWGLNRESITTRRRRSKAAV

SEQ ID NO: 304 - protein, 65 aa, Marinobacter hydrocarbonoclasticus
MPYVNIKVTDDNVSVEQKRQLIQRTTQLLVDVLNKNPDATHVVIDEVSTDNWGVKGETVTNLRKR

SEQ ID NO: 305 - protein, 74 aa, Marinobacter sp. BSs20148
MPYVNIKVTDEGVTSEQKQALIAGATKLVVDVLNKSPTTTFVIIEEVNTDNWGIGFEQVTELRRQASTSPADKT

SEQ ID NO: 306 - protein, 71 aa, Methanosphaerula palustris; identical to SEQ ID NO: 86

SEQ ID NO: 307 - protein, 71 aa, Methylomonas methanica
MPLITVNLIENVFSSEQKAEIIEKLTDTMVAIEGENMRQVTWVKIEEVPEGQWGIGGTPLTAAMVHQIQKG

SEQ ID NO: 308 - protein, 63 aa, Methyloversatilis universalis
MPFAQINLIEGRTEDQKRAVIEKVTQALVEAVGAPRETVRVMIVEVPKSHWGIGGVSAKDLGR

SEQ ID NO: 309 - protein, 68 aa, Mycobacterium sp. 141
MPLVEVTLAAGRSDDQIRAMIHEVHEAVLRTVNTQPEHIRVIVREVPRTHWATGDITIAEMDRKKGSA

SEQ ID NO: 310 - protein, 72 aa, Myxococcus stipitatus; identical to SEQ ID NO: 87

SEQ ID NO: 311 - protein, 59 aa, Neisseria meningitidis
MPYVNIKVTGGKEAPTAAQKAELIGGVTELLARVLGKNPETTVVVIDEVDTDNWGIGGK

SEQ ID NO: 312 - protein, 72 aa, Niastella koreensis
MPFVKILVTDEGVTREQKRQLIQGVTDVISDVLNKDPQLTHIAIEEVNTDNWGWAGEQISILREQGITADKK

SEQ ID NO: 313 - protein, 62 aa, Nitrosomonas eutropha
MPYVNIRLAGTLTNEQKQQIATEITDTLERIAHKPKSYVYIAFDEMPHESWAIGGRLLGSDK

SEQ ID NO: 314 - protein, 62 aa, Nitrosomonas sp. AL212
MPYVNIRVAGTLTREQKQQIATELTDTLERVANKPKSYTYISFDELPDENWAVAGKLLGGED

SEQ ID NO: 315 - protein, 73 aa, Phaeobacter gallaeciensis
MPYVNIKVTQEGGPNGIGPSAEEKAQLVEGVTDLLQRVLNKDPSTTFVVIDEVPLDNWGIQGRPVSHIRAAQS

SEQ ID NO: 316 - protein, 73 aa, Phycisphaera mikurensis
MPYVNVRITEGATRGQKAAVIADVTRALVEHRGKKPEHCHVVIDEVAEANWGFAGQLTDDWKREPQAEDAGGS

SEQ ID NO: 317 - protein, 61 aa, Planococcus donghaensis
MPYVTVKMLEGRTEDQKRALVEKVSEAVSETTGAPIENVTVFIEDMKKSNYGTKGKLFSDQ

SEQ ID NO: 318 - protein, 66 aa, Pseudanabaena sp. PCC 7367
MPYVNVQITRGASRSQKAEIVKQITNTLVSTLGKQPEHIHIVIQEIEEEDWGFAGLLTDDWKKQNQ

SEQ ID NO: 319 - protein, 70 aa, Rhodopirellula baltica SH 1
MPYINVQITKGATREHKSRLVRRMTDALVEELGKKPEYIHIVIQEVDDEDWGYAGMLTDEWKRQQQSQQQ

SEQ ID NO: 320 - protein, 73 aa, Roseobacter denitrificans
MPYVNIKVTQEGGPQGTGPSAQEKARLVAGVTELLFEVLGKNPATTFVVIDEVSLDNWGMDGKQVRTIRAAQG

SEQ ID NO: 321 - protein, 68 aa, Ruegeria pomeroyi
MPYVNIKITREGATPAQKAELIAGVTDLLKQVLNKNPETTFVVIDEVEMEHWGIAGLPVAEYRQRKAG

SEQ ID NO: 322 - protein, 73 aa, Ruegeria sp. TM1040
MPYVNIKVTQEGGPNGTGPSTEEKQKLVAGVTDLLASVLGKNPATTFVVIDEVPLENWGLEGRQVSTIRAEQG

SEQ ID NO: 323 - protein, 75 aa, Serratia marcescens
MPFVNVQTVKGMLDERQKSELLSRITDLLVEIEGRGDPAFRQSVWVRIDEHAPEQWSLGGVKPTAAMIAAKFPPR

SEQ ID NO: 324 - protein, 69 aa, Shewanella denitrificans
MPYINVKITDEGVSNEQKQAIIRGCTELMVKVLNKNPATTFVVIEEVSTDNWGIGFDQVTELRKRASQT

SEQ ID NO: 325 - protein, 76 aa, Sphingobium sp. SYK-6
MPYVKIEVIEGISREQKAGLVADVTRSLVDRLGKRPEQVFVVIQEVSADDWGYAGALISERTTPRREDASAAVPSQ

SEQ ID NO: 326 - protein, 73 aa, Sphingopyxis alaskensis
MPYVNIRITREGATAAQKAELIAGVTDLLQEVLAKNPATTVVNIDEVDLDNWGVGGLPVAAYRARQKSKEPET

SEQ ID NO: 327 - protein, 62 aa, Staphylococcus sciuri
MMPIVNIKLIEGRSDEQLKGLVKDVTEAVHQNAKAPKENIHVIIEEMKPEHYGVAGTRKSDS

SEQ ID NO: 328 - protein, 61 aa, Streptomyces pristinaespiralis; identical to SEQ ID NO: 73

SEQ ID NO: 329 - protein, 61 aa, Sulfuricella denitrificans
MPYVNIRIAGSLTREQKQKIAEEVTDTLERIANKPRSYTVISFDEMPYENWAFAGELLDNN

SEQ ID NO: 330 - protein, 67 aa, Terriglobus roseus; identical to SEQ ID NO: 88

SEQ ID NO: 331 - protein, 66 aa, Turneriella parva
MPYVNIQITKGATREQRSELVKDVTASLVRILGKKPEHTHIVIQEIEEDHWGFAGMLTEDYKKRNP

SEQ ID NO: 332 - protein, 64 aa, Vibrio fluvialis; identical to SEQ ID NO: 24

SEQ ID NO: 333 - protein, 71 aa, Vibrio orientalis
MPYINVKVTDEQVTVEQKQEIIQAMTQVMIDVLDKDPATTFVVIDEVNTDNWGIGFDQVSQLRQHASLHKQ

SEQ ID NO: 334 - protein, 67 aa, Wolinella succinogenes
MPYVNIKITQEGATPEQKERLIQGVTELLQKELGKNPATTVVVIEEVPTDNWGVGGESITKRRAKGV

SEQ ID NO: 335 - protein, 78 aa, Achromobacter piechaudii
MPYVNIKVTREGTAPGATATTPEQKRALIKGVSDLLFEVLGKPHASTFVVIDEVDMENWGVGGVTVPEYRAQQAKVDK

SEQ ID NO: 336 - protein, 78 aa, Acidovorax sp. CF316
MPYVNIKVTREGTAPGASATTPEQKRALIQGVSDLLYEVMGKPQDSTFVVIDEVDLDNWGVGGLTVPEWRARSKAQPG

SEQ ID NO: 337 - protein, 67 aa, Acidovorax sp. CF316
MPYVNIQVTREGVTREQKAELIAGVTELLVRVLHKNPATTHVVIDEIELDNWGVGGLPVAEFRKRQP

SEQ ID NO: 338 - protein, 68 aa, Acinetobacter sp. NIPH 298
MMPYILIQVTDEGVTVAQKQQLIRGATDLVVDVLNKDPATTFVVIDEVATENWGVAGESVQQMRQRIR

SEQ ID NO: 339 - protein, 69 aa, Alicyclobacillus acidocaldarius
MPLVQVTMVEGRSPEQKRALMEKLTDAVVETLGAKRETVRVILYEVPKAHWGVGGVSKAEQDAARGESP

SEQ ID NO: 340 - protein, 71 aa, Anaerotruncus sp. G3(2012); identical to SEQ ID NO: 61

SEQ ID NO: 341 - protein, 62 aa, Azospirillum lipoferum
MPTINIQLFEGRTVEQKRDYAKALTEATVKVLGCPPSAVDIIFQDVKKSDWASGGELWSDKS

SEQ ID NO: 342 - protein, 61 aa, Bacillus sp. B14905
MPYVTVKMLEGRTEEQKRALVKEVTEAVSRTVNAPAENVTVFIEEMSKNHYGVAGIRLSDK

SEQ ID NO: 343 - protein, 61 aa, Bacillus pseudofirmus
MPIVTVQLLEGRTDDQKRALVEKVTDAVSETTGAPAERISVIIDEMKPTNFAVNGKRLSDQ

SEQ ID NO: 344 - protein, 69 aa, Beggiatoa alba
MPYINIQVIQGITREQKQQLVQEITDSLVRILGKKPEHTHIVIQEIAEENWGFAGCLTDDWKKQQLNKQ

SEQ ID NO: 345 - protein, 59 aa, beta proteobacterium KB13
MPYINVKLAGSINKEQKAEIAKEITDTMSRIANKPASYTYVVFDEVATEDWAIGGNLLG

SEQ ID NO: 346 - protein, 72 aa, Brevundimonas diminuta
MPYVNIRITREGATREQKAQLIAGVTDLLYEVLDKPPAVTFVVIDEVPLEDWGVGGLPVDEYRQSLAAAPKS

SEQ ID NO: 347 - protein, 64 aa, Burkholderia gladioli
MPTFHVDLFEGRSAEQKRRFIEAVTRVTCETLDCPPSAVDIILHEVKKENWATGGKLWSDADGG

SEQ ID NO: 348 - protein, 62 aa, Burkholderia nodosa
MPTFHIEMFEGRSVEQKRQLAAALTETTCKVLGVEPQSVDIIFHDVKPENWATAGKLWSDPR

SEQ ID NO: 349 - protein, 77 aa, Burkholderia nodosa
MPIVTIQVTREGTRPGADSVTAEEKAQLIKGVSQLMLDVLNKPLEATFVVIEEVPTENWGWGGLPTDVYRRQKGLIK

SEQ ID NO: 350 - protein, 69 aa, Campylobacter gracilis
MPFVNIKVAAPEPTKEQKKQIIAEITNTLARVLGKDPAAILVMIETLGMDSIGKSGLSLEEIKQNKEKK

SEQ ID NO: 351 - protein, 70 aa, Catenovulum agarivorans
MPIVTIQVTDEGVSKAQKAELIKRSTEMLSQVLDKDPQTTFVVIEEVNLDNWGVAGLPVMQFRQQHLLEK

SEQ ID NO: 352 - protein, 76 aa, Cedecea davisae
MPHIEIKCFPRELSEEQKQAIASEMCDVLKKHFGSKDESLSVALKMIEQARWKEEVWDTQLAPEMDSLLKKPGYSL

SEQ ID NO: 353 - protein, 68 aa, Clostridium ultunense
MPFITIKMLEGRSPELKKELVEKVTAVVAETLAVPKDRIHLFLEDMKPDTYGHGGVLASERNKTPETK

SEQ ID NO: 354 - protein, 70 aa, Coleofasciculus chthonoplastes; identical to SEQ ID NO: 46

SEQ ID NO: 355 - protein, 76 aa, Cronobacter pulveris
MPHVDVKFFPRGLSEEAQQALAAELSDVIVKHLQSKESSVSVALEEVQPDDWKAQVWDKEIAPNLDKLAKKPGYSM

SEQ ID NO: 356 - protein, 71 aa, Cyanobium sp. PCC 7001
MPYVNVQITKGATREQKAQLVKDITDSLVAVLGKKPEHTHVVIQEIEYEDWGFSGLLTDEWNKGQVTAADA

SEQ ID NO: 357 - protein, 66 aa, Cyanothece sp. ATCC 51142
MPYINIQITTGATKEQKAQLVREVTDSLVRILGKKPEHIHIVIQEIEEENWGFCGLLTDEWKKQQN

SEQ ID NO: 358 - protein, 70 aa, Cystobacter fuscus; identical to SEQ ID NO: 91

SEQ ID NO: 359 - protein, 76 aa, Cystobacter fuscus
MPYINVKFTKGSATPEQKARIIEGMTQVLREVIDKAPSATFVVIDEVEYEDWGIGGLPVLEYWKRTGKTASASEES

SEQ ID NO: 360 - protein, 73 aa, delta proteobacterium MLMS-1; identical to SEQ ID NO: 77

SEQ ID NO: 361 - protein, 67 aa, Desulfococcus multivorans
MPYVNIKVTREEVTAEQKAGLIKGVTTLLTEILGKNPETTVVVIEEIDTDNWGIGGEPVTVRRRRGQ

SEQ ID NO: 362 - protein, 67 aa, Desulfovibrio oxyclinae
MPYVNIRITREGATAEQKQQLMDGVTALLRDVLGKNPETTFVIIDEVDTDNWGIAGESVTNRRKRGR

SEQ ID NO: 363 - protein, 67 aa, Dickeya zeae
MPFVNIRITKDGASAEQKKQLIAGVTQLLVDTLGKNPATTVVIIDEVETDNWGIGGQSVTERRQPQS

SEQ ID NO: 364 - protein, 60 aa, Enterobacter lignolyticus; identical to SEQ ID NO: 92

SEQ ID NO: 365 - protein, 61 aa, Exiguobacterium oxidotolerans
MPYVTVKMLSGRTVEQKRALIEKVTEAVSETVNAPKENITVFIEEMEKTDYGQAGVMFADK

SEQ ID NO: 366 - protein, 67 aa, Fulvivirga imtechensis
MPYINIKVTDELVTREQKQKLIEGITRLMVEVLNKNPDTTHVVIDEVPIDNWGLSGRQYSEIKKYKK

SEQ ID NO: 367 - protein, 66 aa, Gallaecimonas xiamenensis 3-C-1
MPYVNVQITAGATREQKTQLVQDITASLSRVLGKKPEHTHIVIQEIAEENWGYAGQLTDDWKKQQP

SEQ ID NO: 368 - protein, 68 aa, Hahella chejuensis
MPYVNIKITREGATAEQKAALIEGVTRLLQETLNKNPATTFVVIDEVDTDNWGIGGESVTVRRQRDTG

SEQ ID NO: 369 - protein, 64 aa, Halomonas boliviensis
MPIVTIQQFPRDVEQKRELAKRITTAFTEVYGTDPQSVQVFFQEINGENWSKGGELGIGRTKPK

SEQ ID NO: 370 - protein, 63 aa, Halomonas anticariensis
MPIVTIQQFPRPLEQKRELARRITEAFVEVYGTKEEAVQVFFSEIDGEGWAKGGVMGCDKTDK

SEQ ID NO: 371 - protein, 63 aa, Halomonas sp. BC04
MPIVTIQQFPRDLAQKRELARRITEAFAEVYGAKPESVQLFFSEVDGQSWAKGGVMGCDSTPG

SEQ ID NO: 372 - protein, 70 aa, Helicobacter fennelliae
MPFVNIKITRENGEPTQTQKQQLISGVTELLSKVLNKNPQTTIVVIDEVETDNWGIGAESITERRKRQKK

SEQ ID NO: 373 - protein, 58 aa, Helicobacter fennelliae
MPLVTIKLAKPELDKAQKEQLIADITELLSTKYGKAKERIVVCLESIEPSDIGFGGIE

SEQ ID NO: 374 - protein, 71 aa, Kutzneria albida
MPFANLKVPAGLLTAEQKEDLITKVTDLYAETFGDRVRDNTMVLVDEVADGGWGIRGEVLTLAKLQRHGES

SEQ ID NO: 375 - protein, 70 aa, Labrenzia alexandrii DFL-11
MPYVHIQVTDEGVSKEQKAELIKGATDLLVDVLNKEPATTFVVIEEVPLENWGIAGVPVTEFRARRKTGT

SEQ ID NO: 376 - protein, 62 aa, Lactococcus raffinolactis
MPFVHVDLFEGRTDEQKIALAHDITEVVVKNTGAPKSAIHVFINDMKEGTYFPQGEIKKANQ

SEQ ID NO: 377 - protein, 68 aa, Leptolyngbya sp. Heron Island J
MPYVNIRITREGATPQQKTALIKGVTDLLVTVLNKSPNTTFVVIDEVATEDWGIAGMPVHEFRNPTQK

SEQ ID NO: 378 - protein, 66 aa, Leptonema illini
MPYINLQITKGASRDQKKKIVEEFTDTLVRVLGKKPEHTHIVIQEIEEEDWGFAGMLTDDYRKQHQ

SEQ ID NO: 379 - protein, 59 aa, Leptospira kmetyi
MPYVNLQVAGPLTRQQKEEIVKEFSETLHRVAGKPPAVTYIVIEEVAKENWAVGGKLLE

SEQ ID NO: 380 - protein, 66 aa, Lonsdalea quercina; identical to SEQ ID NO: 93

SEQ ID NO: 381 - protein, 72 aa, Marinomonas sp. MED121
MPYINVKITDENVTREQKQAIIKGCTQIMVDVLDKDPQVTFVVIDEVKLDDWGIGFDQVSKLRENTLKSPLK

SEQ ID NO: 382 - protein, 70 aa, Martelella sp. AD-3
MPFVNIKTPEAALTNAQKAEIGHRVTDMLVEYFTEAARVHTMVLIEEVKDGGYYRADETFVIPEDYRQKE

SEQ ID NO: 383 - protein, 64 aa, Mesorhizobium amorphae
MPFVNIRILEGHSQKRKDEISRRVTDAISEVVELPREAIWVVFEDVAASDWFVGGTAVGKPKSQ

SEQ ID NO: 384 - protein, 71 aa, Mesorhizobium sp. LNHC232B00; identical to SEQ ID NO: 94

SEQ ID NO: 385 - protein, 78 aa, Mesorhizobium sp. LSHC422A00
MPIVTIQVTREGTKPGTTSVTAEEKAALIKGASYLLRDVLNKSLDSTFVVIEEVDTENWGWGGLPTLDYRRTRAATGV

SEQ ID NO: 386 - protein, 59 aa, Methylophilales bacterium HTCC2181
MPFINVKLAGALNKDQKQQIAKEITEMMQRVANKPASYTYIVFEEVKTEDWAIGGALLG

SEQ ID NO: 387 - protein, 67 aa, Microscilla marina
MPYINIKVTDENVTKEQKQQLIAGATRLVVEVLNKNPATTHVVIDEVHIDNWGVKGKQYSELINSQN

SEQ ID NO: 388 - protein, 65 aa, Mycobacterium intracellulare; identical to SEQ ID NO: 13

SEQ ID NO: 389 - protein, 63 aa, Neisseria wadsworthii; identical to SEQ ID NO: 51

SEQ ID NO: 390 - protein, 65 aa, Neptuniibacter caesariensis
MPYVNIKITDDGVTQAEKKALIQGTTKLLQEVLNKDPQSTFVVIDEVDTDNWGIGYETVTEIRKG

SEQ ID NO: 391 - protein, 61 aa, Paenisporosarcina sp. TG-14
MPYVTVKMLEGRTDDQKRALVEKVTAAVVETTNAPIENVVVFIEDMPKNHYAVAGKLFSDN

SEQ ID NO: 392 - protein, 61 aa, Pantoea sp. At-9b
MPEIIVHMVSGRTPEQKKKLMDGIYQAVKDALPVKEEYIVISLIETDKEHKSRGGVLFKDL

SEQ ID NO: 393 - protein, 64 aa, Pelosinus fermentans; identical to SEQ ID NO: 95

SEQ ID NO: 394 - protein, 69 aa, Providencia alcalifaciens PAL-1
MPYINIKVTDEGVTNDQKKRLIEGATTLLNQVLGKPKSSVFVVIDEVKTDNWGVGGEIVTDLRKNENSR

SEQ ID NO: 395 - protein, 67 aa, Pseudoalteromonas rubra
MPYIHIRVTDEGVTNAQKQSLIKGTTQLMVDVLGKSPETTFVVIDEVSTDNWGIGYEQVTAIRQRQT

SEQ ID NO: 396 - protein, 76 aa, Pseudoalteromonas spongiae
MPYVNIQVTKEGGTHNVGPTDEQKRLLIAGVTELLQNVLNKDPKTTHVVISEVALANWGVGGVPVIDYRAQNKESI

SEQ ID NO: 397 - protein, 70 aa, Pseudoalteromonas sp. NJ631
MPIINIKITKEGVTPAQKQALIKGATQLMVDVLDKAPQVTFVVIEEVETDDWGIGFDQVSMLRQQHLSPK

SEQ ID NO: 398 - protein, 66 aa, Pseudogulbenkiania ferrooxidans
MPYVNVQITAGATREQKAALVKDITDSRVRHQGKKPELTHIVIQEIAEEDWGYAGQLIDDWKRGQG

SEQ ID NO: 399 - protein, 66 aa, Pseudomonas sp. M1; identical to SEQ ID NO: 96

SEQ ID NO: 400 - protein, 71 aa, Pseudomonas brassicacearum
MPFISVRITRDGVTQEQKAQVIAEITRTMQTVLDKDPRLTHIVIDEVDTDSWGYAGMTTTQYRKQLAESKS

SEQ ID NO: 401 - protein, 69 aa, Rhizobium sp. CF142
MPFANYKFPEGTLTAEQKEDIILRTTAMFVDFFGEGVRPYTMVLVDEVVDGGYGRAGETLTIAKMQKGR

SEQ ID NO: 402 - protein, 78 aa, Sphingobium japonicum
MPFVDIRLAGAVTRAQKAALVADITQSLVERLGKPAAAVQINITELSLENYAAGGQLLADRAPAPSVREDAIAADTPR

SEQ ID NO: 403 - protein, 77 aa, Sphingobium ummariense
MPIVTIQVTREGTVAGADRTTSEQKAAIHKGIADLLQDVLGKDPEDTFVIFQEVELEDWGRGGLAVPAWRAARNTPG

SEQ ID NO: 404 - protein, 76 aa, Sphingomonas wittichii RW1
MPFVDIRIAGTATRAQKSAIVADVTKALGAHLGKPASAVQVNIIEVSTENYGAGGILIADRPASPAQEVASADGKR

SEQ ID NO: 405 - protein, 73 aa, Sphingopyxis sp. MC1
MPYVHIRITREGASAEAKRELIAGVTDLLQRVLGKNPATTVVTIDEVDMDNWGIGGLPVGEFRAAQAQQETQR

SEQ ID NO: 406 - protein, 69 aa, Sporosarcina newyorkensis; identical to SEQ ID NO: 97

SEQ ID NO: 407 - protein, 64 aa, Staphylococcus caprae
MLYMPIINVKLLEGRTDEQLKNLVTEVTHAVEKTTGANKEAIHVVIEEMEKSHYAVGGVRKSDQ

SEQ ID NO: 408 - protein, 62 aa, Streptomyces turgidiscabies; identical to SEQ ID NO: 98

SEQ ID NO: 409 - protein, 70 aa, Streptomyces sp. Mg1
MPVITVNWWKGNDRAARGDLVAGITDVVTRVSGCPRESVTVIVRDVELGHWGKGGRLADDRGPAGPRSSQ

SEQ ID NO: 410 - protein, 72 aa, Streptomyces coelicoflavus ZG0656
MPFAHFKVPAGTLSAEDKKKIVERTTDLYAEIYGERARPTTVVLVDEVPDGGWGVAGHVLTAAMLNGESPGD

SEQ ID NO: 411 - protein, 59 aa, Thaumarchaeota archaeon SCGC AB-539-E09; identical to SEQ ID NO: 99

SEQ ID NO: 412 - protein, 72 aa, Thiobacillus thioparus
MPFINIKIIEGVFTDAQKQEIIKKMTDTMVSIEGENMRGVTWTVIEEVKSGDWGIGGKGLTTEDVKSLAAGK

SEQ ID NO: 413 - protein, 68 aa, Thiorhodospira sibirica
MPYVNIKITREGVTQAQKKQLIAGATQLLVDVLGKNPATTVVVIDEVEMENWGVGGIPVDERRAQERR

SEQ ID NO: 414 - protein, 60 aa, uncultured bacterium esnapd10
MPYIDVKLFESRLTEATEKELIDRITAAVVDTFGEDIREQTWVSLTGIPAHRWGVGGIRH

SEQ ID NO: 415 - protein, 61 aa, uncultured Desulfofustis sp. PB-SRB1
MPYVNIRVAGTLTTAQKATISKGVTEVISQAAGKPPESVLIFIDEATRDNVAKAGKLLSQS

SEQ ID NO: 416 - protein, 71 aa, Vibrio coralliilyticus
MPVVTIQQSPGRSKAQKQLLLQRITEAFEEAYGTAPEAVTVFFQNFDEEHWGKGGQLHADRATSPQSNVKT

SEQ ID NO: 417 - protein, 73 aa, Xenococcus sp. PCC 7305
MPFVTIQIAKGHSLEKKRELAQAVTDTLVSNLGTKPEWVTIHIDEFDRENWAVGGQLHVDKHSGRHSEKVISN

SEQ ID NO: 418 - protein, 70 aa, Acinetobacter haemolyticus
MPFVSVKVTGGNEAPTVEQKKLIIEGITQLLSTVLNKNPATTMVIIDEIEMDNWGVAGKTTTERRQEAQK

SEQ ID NO: 419 - protein, 70 aa, Acinetobacter baumannii 1437282
MPYVNIKITDENVTVENKRALIEGVTELLYQVLAKPHATTFVVIDEINTDNWGVGGQSVTEFRQKTSRKE

SEQ ID NO: 420 - protein, 71 aa, Actinokineospora inagensis; identical to SEQ ID NO: 100

SEQ ID NO: 421 - protein, 67 aa, Actinokineospora enzanensis; identical to SEQ ID NO: 70

SEQ ID NO: 422 - protein, 67 aa, Actinomycetospora chiangmaiensis; identical to SEQ ID NO: 89

SEQ ID NO: 423 - protein, 76 aa, Advenella mimigardefordensis; identical to SEQ ID NO: 101

SEQ ID NO: 424 - protein, 74 aa, Aestuariimicrobium kwangyangense
MPFINVKVIENVFTPEQTQQIVRGLTDAMVAIEGENMRPVTWCVVEEVKSGSWGIAGNPLSTEDVKALAKGGPA

SEQ ID NO: 425 - protein, 63 aa, Afifella pfennigii; identical to SEQ ID NO: 102

SEQ ID NO: 426 - protein, 67 aa, Agrobacterium tumefaciens; identical to SEQ ID NO: 90

SEQ ID NO: 427 - protein, 63 aa, Alicyclobacillus acidoterrestris
MPLIQMSILEGRSKEAKKAAMKEVTDAVVRTLGVPPETVRVLIYEMPKEHWGVGGISKADESN

SEQ ID NO: 428 - protein, 63 aa, Alicyclobacillus contaminans
MPLVQISILEGRPQEAKKRLIEAVTEAVVHTLDANPGAVRVLLYEVPAAHWGVGGVTKADSSK

SEQ ID NO: 429 - protein, 63 aa, Alicyclobacillus herbarius; identical to SEQ ID NO: 78

SEQ ID NO: 430 - protein, 61 aa, Amphibacillus jilinensis
MPYVTVKMLEGRTDQQKKELVEKVTDAVVETTGANREKVVVFIEDMKHDHYAVGGKRLSDQ

SEQ ID NO: 431 - protein, 61 aa, Amycolatopsis nigrescens; identical to SEQ ID NO: 79

SEQ ID NO: 432 - protein, 67 aa, Aquimarina latercula
MPYINIKVTDEQVTKEQKRLLIVGATQLLADVLDKNPKTTHVVIEEIPIDNWGVDAKQYSNSEKFKS

SEQ ID NO: 433 - protein, 64 aa, Aquimarina macrocephali
MPYINIKVTDEGLTKRQKQQLIAGATQLVVDILNKNPETTHVVIDEVPQDNWGVRGKQYSELNK

SEQ ID NO: 434 - protein, 63 aa, Arcobacter sp. L
MPIATINIIEGRSDEKKEKLIEKVSLAIAESLDAPLESVRVIINEMPKQHFGIGGKSVKKLGK

SEQ ID NO: 435 - protein, 69 aa, Arcobacter cibarius
MPLINVKMTHEDGGATKEQKEELAKELTNAFVKIFNRGQNSTVVIIDEVNTDNYAIGGKTITKIREEQK

SEQ ID NO: 436 - protein, 71 aa, Arcobacter sp. AF1581
MPVINVKITKEDGGATIEQKEELSKGITELFSKIFNRSASNAVVLIEEFNTDNYYIGGKSITKIREEQKSK

SEQ ID NO: 437 - protein, 62 aa, Azospirillum brasilense
MPLINVQLFEGRTLEQKRAYAKALTDASVAVLGCSPEAVDVIFHDVKKSDWASGGKLWSDPE

SEQ ID NO: 438 - protein, 61 aa, Bacillus cellulosilyticus
MPIVTVKLLEGRSDDQKRALVEKVTAAVSETTNAPAEKVAVIIEEMSPNHYGVAGKRVSDS

SEQ ID NO: 439 - protein, 64 aa, Bacillus clausii
MPIVTVQLLEGRTDEQKRALVEKVTDAVSETTGAPKERVSIIISEMKPNDYGVAGKRPSDQHEQ

SEQ ID NO: 440 - protein, 63 aa, Bacillus aurantiacus
MPYVTVKMLEGRTDDQKRELVQKVTDAVSESTGAPREKVAVIIEEMSPAHYGVNGERISDIMP

SEQ ID NO: 441 - protein, 61 aa, Bacillus halodurans
MPIVTVKFLEGRSDEQKRALVERVTEAVAETIQANPEKIHVVLEEMRKDHYGVAGKRISDQ

SEQ ID NO: 442 - protein, 65 aa, Bacillus sp. URHB0009
MPIIHVHLLEGRSKQLKQQLIAEVTSAVSKTLGNSPESVRVLLHEVPEENWGVAGSSISQRKRES

SEQ ID NO: 443 - protein, 61 aa, Bacillus sp. ZYK; identical to SEQ ID NO: 62

SEQ ID NO: 444 - protein, 61 aa, Bacillus sp. L1(2012)
MPIVTVKMLEGRSDEQKRALVEKVTEAVSETIQAPPERISVVIEEMKKTNFGVAGKRASDV

SEQ ID NO: 445 - protein, 61 aa, Bacillus sp. UNC438CL73TsuS30
MPIITVKMLEGRSDEQKRALVEKVTEAVSETAGAPKESISIIIEEMSKNHYAVAGVRAIDK

SEQ ID NO: 446 - protein, 61 aa, Bacillus sp. UNC41MFS5
MPYVTVKMLEGRTDDQKRALVTKVTDAVSETTGAPKEAIHVMIEEMSKSNLGIAGVRKSDQ

SEQ ID NO: 447 - protein, 67 aa, Bacillus sp. L1(2012)
MPFIQINILEGRTPEKKERLIREVSDLVADVLDAPIESVRILIQEMPSEHWGIAGESVKKRKVNAKG

SEQ ID NO: 448 - protein, 62 aa, Bacillus smithii; identical to SEQ ID NO: 103

SEQ ID NO: 449 - protein, 61 aa, Bacillus chagannorensis
MPIVTVKMLEGRTDEQKRELVEKMTEAVTSTTGADAERVSVVIEEMQPQNFGKAGVRASDQ

SEQ ID NO: 450 - protein, 69 aa, Bacillus chagannorensis
MPYVNIKVTHEHGGLTKEQKHELIEGVTDLLSEKLQKNPASTVVVIDEIDTDNYGLGGEQITERRKKES

SEQ ID NO: 451 - protein, 67 aa, Balneatrix alpica
MPYVNIKVTREGVSREQKQALISGVTQLLVEVLGKNPATTVVVIDEVELDNWGVAGELVSDRRAQGR

SEQ ID NO: 452 - protein, 68 aa, Balneatrix alpica
MPYVNIKVTKEGLSKAKKEALIKGTTQLLVEVLDKNPATTFIVIDEVDTDNWGIGYDQVTTLRQAKQD

SEQ ID NO: 453 - protein, 62 aa, Bordetella sp. FB-8; identical to SEQ ID NO: 4

SEQ ID NO: 454 - protein, 69 aa, Bordetella trematum
MPYVNIQITREGVTTTQKAELIRRTTAMLEEVLNKNPATTFVVIQEIDTDDWGVGGETVTVRRARDAQA

SEQ ID NO: 455 - protein, 78 aa, Bradyrhizobium elkanii
MPIVTIQVTREGSAPGRDSVSSEEKAELIAGVSQLLLKVLNKPLEATFVIIEEVETENWGWGGLPVPEFRKRRAAGGT

SEQ ID NO: 456 - protein, 63 aa, Burkholderia sp. BT03
MPIIQVYMMEGRSDEQKTKLIEALTSATVEQVGAPRESVRVLISEMPKTHWGIGGKTAKELGR

SEQ ID NO: 457 - protein, 76 aa, Burkholderia acidipaludis
MPIVTIQITREGTTPGTDHVTAEEKAKLIHGVSRLLLDVLNKPLDATFVVIEEVEMENWGWGGLPVPEYRKQRAMK

SEQ ID NO: 458 - protein, 78 aa, Burkholderia kururiensis
MPIVTIQVTREGTKPGRESVTAEEKARLIHGVSHLLLDVLNKPLESTFVVIEEVDTANWGWGGLPVQAYRRQRAAQQP

SEQ ID NO: 459 - protein, 78 aa, Burkholderia sp. UYPR1.413
MPIVTIQVTREGNTPGTSAVTPEEKAQLIKGASELLLNVLNKPLEATFVVIQEVEKENWGWGGLPVDEYRKLKAQKKD

SEQ ID NO: 460 - protein, 67 aa, Campylobacter mucosalis
MPYVNIKLAGPEPTDEQKEQVIAEVTEVMERVLGKKKERVMVMIETLSPQSIGIGGKSVKTITKESK

SEQ ID NO: 461 - protein, 70 aa, Campylobacter mucosalis; identical to SEQ ID NO: 104

SEQ ID NO: 462 - protein, 70 aa, Campylobacter cuniculorum
MPIVTIKLAKPAMSKEQKAALISDITELLNKKYNKAKERIVIMIEESEPFDIGFGGESVESIKAKEAKNE

SEQ ID NO: 463 - protein, 67 aa, Campylobacter sp. CIT045; identical to SEQ ID NO: 105

SEQ ID NO: 464 - protein, 71 aa, Candidatus Competibacter denitrificans Run A D11
MMPYVNIKITREGGTTAAQKAALIEGATELLVRVLGKSPATTVVVIDEVDMENWGIGGLPVEDYRKRHAGQ

SEQ ID NO: 465 - protein, 71 aa, Castellaniella defragrans 65Phen
MPYVNIKITREGATAEQKARLIEGATQLLVDVLGKNPKTTFVVIEEVDTDSWGIAGETVTTIRARAKASGQ

SEQ ID NO: 466 - protein, 58 aa, Catellicoccus marimammalium
MPFIHVELLEGRTPEQKKALIEDITEVVSKDADVPKERIHVIIRDMKHGDYGCGGEVK

SEQ ID NO: 467 - protein, 68 aa, Catelliglobosispora koreensis
MPYVNVKITREGATAAQKAEIIKGVTQLLVDVLNKNPATTFVVINEVELEDWGVGGLPTVGYRKSLES

SEQ ID NO: 468 - protein, 78 aa, Caulobacter sp. URHA0033
MPMVTIQVTRQGSAPGRDAVTAEEKAALIAGASQLLLEVLGKPLETTFVVIEEVPLENWGVGGLPVAEFRRRRASHGE

SEQ ID NO: 469 - protein, 73 aa, Chrysiogenes arsenatis
MPYVNIKVTKEGLTVAKKEELIRGVTQLLVDTLGKNPATTVVVIDEVDMDNWGVGGERVSVIRERQQLQQSQQ

SEQ ID NO: 470 - protein, 69 aa, Clostridium ultunense Esp
MPYVNIKVTKEKIPVTAEQKERLIQGVTRLLQEVLGKNPKTTVVVIDEVDTDNWGIGGESVTIRRKRGE

SEQ ID NO: 471 - protein, 61 aa, Cobetia crustatorum
MPMIRAEFISGKSEEQKRELIEALTRETARVLGVREESVWVVLQEVEAENWGVAGKRLSDR

SEQ ID NO: 472 - protein, 63 aa, Comamonas testosteroni
MPFAQIYILEGRTPEQKKAVIEKVTQALHEATDAKKETIRVWIHEMPKENWGIAGVSAKDLGR

SEQ ID NO: 473 - protein, 62 aa, Comamonas testosteroni
MPTYRIELFEGRSVEQKRKLAEEITRVTTEVLGSAPESVDIIITDVPRHNWATGGKLWSEQS

SEQ ID NO: 474 - protein, 59 aa, Corynebacterium sputi; identical to SEQ ID NO: 59

SEQ ID NO: 475 - protein, 73 aa, Cucumibacter marinus
MPYVNIKITREGGPTGTGPSAEQKARLIEGVTALLQDVLGKDPKTTVVVIDEVEMDNWGIGGLPVDDFRKQSS

SEQ ID NO: 476 - protein, 63 aa, Dasania marina
MPVITIEMFEGRSLEQKKALVKEITETMERTCNAKPDTVHIIIEDKKMENWGFKGELVSELTK

SEQ ID NO: 477 - protein, 63 aa, Dechloromonas aromatica
MPFAQIYLIEGRTDEQKKLLIEKVSAAMSEALGAPLENTRVWIQDVPKAQWGIGGKTAKDLGR

SEQ ID NO: 478 - protein, 68 aa, Dechloromonas agitata
MPYVNVQITKGASRQQKAEIVKDITDSLVRVLGKKPEHTHVVIQEIADEDWGFAGMLTDDWKAAHSKQ

SEQ ID NO: 479 - protein, 62 aa, Deefgea rivuli
MPYINLRIAGELSREQKQQIVAEFTETMVRVANKPAEYVIVTIDESPLENWGWGGALLDSEE

SEQ ID NO: 480 - protein, 71 aa, Desulfobacter curvatus
MPIVSIKIAKGRTIEQKRNLVQAITDSVVSTLDVKREWVTVLIDELDREDWATGGELHIDKFGAGVGKESK

SEQ ID NO: 481 - protein, 69 aa, Desulfomicrobium baculatum; identical to SEQ ID NO: 106

SEQ ID NO: 482 - protein, 67 aa, Desulfonatronovibrio hydrogenovorans
MPYVNIRITREGATAEQKKELIRGVTKLLQDVLGKNPGSTFVIIDEVDTDNWGIGGEAVTELRKKAD

SEQ ID NO: 483 - protein, 69 aa, Desulfonatronum lacustre
MPYVNIKITREGATAEQKDLLIRGVTRLLADVLGKNPETTVVVIDEVETDNWGIGGVPVTALRRKRKEG

SEQ ID NO: 484 - protein, 65 aa, Desulfospira joergensenii
MPYVNIKITDEGVTKEQKTALIQGATDLLQKVLGKNPKTTFVIIEEVNTDNWGIGGETVTSIRKR

SEQ ID NO: 485 - protein, 65 aa, Desulfovibrio desulfuricans
MPFVNIRITNEGATTEQKQRLIKGVTDLLADVLGKNPKTTFVIIDEVDTDNWGIGGESVTEIRKK

SEQ ID NO: 486 - protein, 67 aa, Desulfovibrio aminophilus
MPFVNIRITRDGATAEQKARLIKGVTDLLVEVLDKNPATTVVIIDEVDTDNWGIGGESVTKRRELAR

SEQ ID NO: 487 - protein, 72 aa, Desulfovibrio piezophilus
MPFVNIRITKEGATADQKQLLIAGVTDLLANVLGKNPKTTFVIIDEVDTDNWGIGGDPVTLLRKDKQHSQDS

SEQ ID NO: 488 - protein, 68 aa, Desulfovibrio desulfuricans
MPFVNIKVTGGAEAPTAEQKAELISGVTELLQRVLHKNPATTVVLIEELSTDNWGIGGESVTVRRRKQ

SEQ ID NO: 489 - protein, 67 aa, Desulfovibrio inopinatus
MPMVNIKITNEGATAEQKAELIKGATELLQRVLGKNPATTIVLIEEIETDNWGIGGESVTVRRNRGQ

SEQ ID NO: 490 - protein, 66 aa, Desulfurispirillum indicum
MPFVNIRITREGATREQKEQLIEGVTNLLVDVLGKNPKTTFVIIDEVETDNWGVGGESVTTLRART

SEQ ID NO: 491 - protein, 72 aa, Desulfuromonas sp. TF
MPFVNVKITSGATAEQKEALIEGITELLSRVLDKNPASTHVVIDEVPPESWGVRGKTVAELRAAGAPGVSKK

SEQ ID NO: 492 - protein, 65 aa, Desulfuromonas sp. TF; identical to SEQ ID NO: 107

SEQ ID NO: 493 - protein, 71 aa, Dolichospermum circinale
MPFVTVQIGKGHSIEKKRKLAQAVTEALVSALGTKPEWITIHIDEFERDNWAVGGVLHSDKHAGRHEETGR

SEQ ID NO: 494 - protein, 66 aa, Dongia sp. URHE0060; identical to SEQ ID NO: 85

SEQ ID NO: 495 - protein, 77 aa, Dongia sp. URHE0060
MPIVNIQVTREGTSPERKSVTAEEKAKLIAGVSQLLLEVLNKPLDSTFVVIQEVELENWGWGGLPVPELRRRKASGS

SEQ ID NO: 496 - protein, 72 aa, Dyadobacter beijingensis
MPFIQIDVTREGVTRAQKQALISGVTDLITEVLDKDPHLTHVVIREVEPDDWGYAGEQVSVLREKGITAQRK

SEQ ID NO: 497 - protein, 78 aa, Dyella japonica; identical to SEQ ID NO: 57

SEQ ID NO: 498 - protein, 78 aa, Dyella jiangningensis
MPFVNIKVTREGTAPGRESVTPEEKAALIKGVSELLLQVLNKPLDSTFVVIDEVEMENWGWGGLPAAEFRKQRSAGKS

SEQ ID NO: 499 - protein, 70 aa, Ensifer adhaerens
MPYVNIKITREGATPEQKATLIKGVTDLLYTVLNKDPATTFVVIDEVALEDWGVGGLPVDAYRRKVGEKS

SEQ ID NO: 500 - protein, 75 aa, Enterobacter aerogenes
MPHVDIKCFPRELSEEQKNALAAEITEVLIRHLSTKDSSVSVALNQVQQSDWQQVWDNEIAPQMDSLIKKPGYRM

SEQ ID NO: 501 - protein, 76 aa, Enterobacter cloacae
MPHIDIKCFPRDLNDEQKSALAADITEVIIRHLNSKESSISVALTEIAPEEWQVNAWDKEIAPQMEKLIKKPGYSM

SEQ ID NO: 502 - protein, 76 aa, Enterobacter lignolyticus
MPHIDIKCFPRGLDDEQKAALAEDIAEVIIRHLNSKDRSISVALNEVAESEWKSQVWDSEIAPQLESLIKKPGYAM

SEQ ID NO: 503 - protein, 76 aa, Enterobacteriaceae bacterium LSJC7
MPHIDIKYFPRELSEEQKQALANDVCEVLKKHLQSKDSALSVALTEVAPEDWKSEVYDPVIKPALDNLVKKPGYTM

SEQ ID NO: 504 - protein, 75 aa, Enterobacteriaceae bacterium strain FGI 57
MPHVDIKSFPHELNAEETEALAADITEVIIRHLHSKNSSVSVAWTQVQPEAWKEVWDSQIGPHMNELIKKPGYSM

SEQ ID NO: 505 - protein, 76 aa, Erwinia toletana
MPHIDIKYFPRELSDEEKSAIAEAMADVLKQHFGSKDSSLSVAMTEIPPEQWKTEVYDPLLKPHLDTLVKKPGYEM

SEQ ID NO: 506 - protein, 75 aa, Erwinia toletana; identical to SEQ ID NO: 108

SEQ ID NO: 507 - protein, 76 aa, Escherichia hermannii
MPHVDIKYFPRELTEEQRQAFADDIAKVVIKHLDSKPTSVSVALEEVQPDNWKQQVWDTDIAPHLDTLAKKPGYSM

SEQ ID NO: 508 - protein, 66 aa, Flexithrix dorotheae
MPYINIKVTDENVTRDQKKKLIEGVTDLMVEVLQKNPATTHVVIDEVPVDNWGVNSKMYSEKFKKS

SEQ ID NO: 509 - protein, 62 aa, Gallionella sp. SCGC AAA018-N21
MPYVHIRLAGTLTREQKAKIAEEITATLERVALKPKEYTYLTFEEVAEENWAIGGKLLDEME

SEQ ID NO: 510 - protein, 64 aa, Geobacillus sp. GHH01
MMMPYVTIKMLAGRTDEQKKALVEKVTDAVVETTGAKRESVVVFIEEMTKDHYAIAGKRLSDTE

SEQ ID NO: 511 - protein, 68 aa, Geobacillus caldoxylosilyticus
MPFIQVYLLEGRSPEKKETLIYELTETVCKVLDAPKENVRVMIQEMPLEHWGIAGESVKKRREYQRSE

SEQ ID NO: 512 - protein, 67 aa, Geopsychrobacter electrodiphilus
MPFVNIKITREGATTAQKAELIKRTTQMLVDVLGKNPARTVVVIEEVETDNWGIGGESITSRRLNGN

SEQ ID NO: 513 - protein, 63 aa, Glaciecola polaris
MPIAQINIIEGRTDEQKEALISEVTDAIVRAIGAPKPSVRIIINEMPKQHFGIAGESVKKLGK

SEQ ID NO: 514 - protein, 60 aa, Gracilibacillus lacisalsi
MPYVTVQMLEGRTDEQRKALIEKVTDAVVETTGASREKVVVFVEDIDKRNYGVGGKRLID

SEQ ID NO: 515 - protein, 68 aa, Hahella ganghwensis
MPYVNIKITNETVSREQKSQLIKGVTKLLVEVLNKKPETTFVVIDEVDLDNWGIGFDQVSELRKVPSD

SEQ ID NO: 516 - protein, 65 aa, Halomonas sp. KM-1
MPIVTIQQFPRDLTQKRELAERITQAFVEVYGVAEESVQVFYNEVDGENWAKAGKMGCDRQQGQG

SEQ ID NO: 517 - protein, 64 aa, Hamadaea tsunoensis
MPMITVDMCAGRTDEQKKALVRQLTDVFLETCGGDRSGVWIVLRDVTTDNWAIGGRLQSDTDPS

SEQ ID NO: 518 - protein, 74 aa, Helicobacter rodentium
MPYVNIKITRENGTPTTEQKQRIIQGVTELLGEVLGRNPASLVVVIDEIDCDNYGVGGITITQKRKLQNPKGIQ

SEQ ID NO: 519 - protein, 70 aa, Helicobacter heilmannii ASB1.4
MPLINIKLAKEETPVTPEQKKALIEGATDLLVKVLNKNRATTVVLIEELTPDSWGIGGQSVCEIRQNAKK

SEQ ID NO: 520 - protein, 63 aa, Herminiimonas sp. CN
MPICQIHLLEGRTVEQKRLLIAKITEAMHEAIGAKPESVRVILAEMPKEHFGIGGKSAGELGR

SEQ ID NO: 521 - protein, 69 aa, Hirschia maritima
MPYVNIKITREGDVTPEQKAKLIQGATDLLSDVLNKDPRTTVVVIDEIKLENWGMGGLPALEYRKTLAD

SEQ ID NO: 522 - protein, 72 aa, Hyphomonas johnsonii MHS-2
MPYVNIRVTREGVTPEQKAELIRGATDLLVRVLNKSPATTVVVIDEVEMENWGIAGLPAEAYREKHRTPASS

SEQ ID NO: 523 - protein, 67 aa, Ignatzschineria larvae
MPYINVKITDKAVTTEQKAAIIDGMTQVLVDVLGKNPATTVVVIDEVPLDNWGIAGIPVVEYRNKSK

SEQ ID NO: 524 - protein, 73 aa, Janthinobacterium sp. CG3
MPYVNIRITRDGVTAEQKQQLIAGTTELLVRVLNKNPDHTFVLIDEVDTDNWGVGYETTTAYRARAAAAKAAG

SEQ ID NO: 525 - protein, 77 aa, Janthinobacterium agaricidamnosum NBRC 102515 = DSM 9628
MPYVNIRITKEGVTPEQKLALIEGATDLLTRVLNKNPATTFVIIDEVETDSWGLNRENITTLRAREARAKAGDKPQD

SEQ ID NO: 526 - protein, 68 aa, Kaistia granuli; identical to SEQ ID NO: 47

SEQ ID NO: 527 - protein, 69 aa, Kiloniella laminariae
MPYVNIKITNEGATKAQKAELIKGVTELLQKVLNKNPATTFVVIDEVEMEHWGVGGLPVEDYRKKISGN

SEQ ID NO: 528 - protein, 64 aa, Kiloniella laminariae
MPIIRVEMFQGRSLEQKRALVKTLTEETARITGCNRDAVYVVIDDVNKENWGAGGILCTDKARH

SEQ ID NO: 529 - protein, 73 aa, Klebsiella pneumoniae
MFHIFAGKPRSRAQKQGLYRRLCARLDDEMAVSPQDVMVVIQFNQLEDWSFTQGRWRMNETGGAARGAAFQMI

SEQ ID NO: 530 - protein, 79 aa, Klebsiella pneumoniae; identical to SEQ ID NO: 109

SEQ ID NO: 531 - protein, 77 aa, Klebsiella sp. MS 92-3
MQTIKGIMTAEQKSELLRRMTDLLVEIEGQGDPQFRQSVWIRIDEHDPEQWSLGGVQPTAAMIAAKFAAARRDGSPE

SEQ ID NO: 532 - protein, 69 aa, Kosakonia radicincitans
MFPRDLNDEQKTALAEDIAAVIMRHLNSKESSISVALNEYAPEEWKAQVWDTEIAPQMEQLIKKPGYSM

SEQ ID NO: 533 - protein, 72 aa, Kyrpidia tusciae
MPLVQLHILEGRSAEVKKRAIEEVTEALHRSLGAPRENIRVVIYEIPKSHWAVGGVTMEEKQPEHVTTSGGS

SEQ ID NO: 534 - protein, 64 aa, Lactococcus garvieae
MPFVHVELVEGRSTEVKKALAKEITESVMKHTGAPREAIYVIYNEMSKENFYPHGEPKNNKKLR

SEQ ID NO: 535 - protein, 73 aa, Lactococcus lactis; identical to SEQ ID NO: 110

SEQ ID NO: 536 - protein, 64 aa, Leptolyngbya sp. Heron Island J; identical to SEQ ID NO: 80

SEQ ID NO: 537 - protein, 63 aa, Leptothrix cholodnii
MPFAQIYLLEGRTEDQKRAVIEKVTAALVEALDAPQANVRVWIHDMPKENWGIAGKSAKDLGR

SEQ ID NO: 538 - protein, 62 aa, Longispora albida
MPMITVEMYPGRTPEQKQALAARLTEAFMETCGSPGQSPDGVWVVIREVPRENWAIGGKMHA

SEQ ID NO: 539 - protein, 67 aa, Marinimicrobium sp. LS-A18
MPVVRVTWFEGKDANAKKAVAKEITESIVKNTQTDPNYIYVIFEDVAQSNWAGAGNLFGEEGEGLKE

SEQ ID NO: 540 - protein, 63 aa, Marinobacterium stanieri
MPIAQLHIMEGRSDEQKETLIKEVTQAIARSLDAPEESVRVLIDEMPKQHFGIGGQSAKKLGR

SEQ ID NO: 541 - protein, 63 aa, Marinobacterium jannaschii
MPIAQINIIEGRSDEQKEALIREITEAIVRAIDAPPQAVRVIINEMPKQHFGIGGESAKALGR

SEQ ID NO: 542 - protein, 68 aa, Marinobacterium jannaschii
MPYVNIKVTNDGVTPQQKQRLIEGTTKLLVEVLDKDPATTFVVIDEVDTENWGIGYDQVSELRKNKGD

SEQ ID NO: 543 - protein, 63 aa, Marinospirillum minutulum
MPIAQIYIMEGRSNEKKEKLIEKITDAMVEALDAPRDVVRVIITEMPKQHFGIGGSSAKKLGR

SEQ ID NO: 544 - protein, 67 aa, Maritalea myrionectae
MPYVHIEVTDEGVTAAQKKALIKGTTDLLQQVLNKDPATTFVVIDEVPLENWGIGGLPVTAFRAQQK

SEQ ID NO: 545 - protein, 70 aa, Martelella mediterranea
MPFVNIKTPEAALTRAQKEDIIHRVTDMLTEYFSEAARPHTMVLIEEVRDGGYGRADEVYVIPEAWRAEG

SEQ ID NO: 546 - protein, 73 aa, Massilia niastensis
MPLVQISIPAGSLDDAKKADMIKRVTDAVVEAEGFPAVRAFTWVHINEVPDGGWGMGGQVVTLDDVAAVLKGS

SEQ ID NO: 547 - protein, 73 aa, Massilia niastensis
MPYILIQVTDEGVTKQQKEQLIAGATDLVVKVLDKDPESTFVVIDEVNTDNWGHGGEMVTLRRQRAAARKQAG

SEQ ID NO: 548 - protein, 61 aa, Methanoculleus sp. MH98A; identical to SEQ ID NO: 111

SEQ ID NO: 549 - protein, 67 aa, Methylophilaceae bacterium 11
MPYINIKVTKEGVTPKQKAELIQGATDLVVKVLNKDPATTFVIIDEVETDNWGVAGETTTARRLRGH

SEQ ID NO: 550 - protein, 67 aa, Methylopila sp. 73B
MPVVRVSWFEGKDHGQKQKVAAEITESIVRNTGTDANYIYVIFDDVAPNDWAGAGKLFGEPTAASST

SEQ ID NO: 551 - protein, 72 aa, Moraxella bovoculi 237
MPYVNIKVTGGTEAPTTGQKAELIQGVTELLGRVLNKNPATTVVVIDEIDMDNYGLGGRSITEVRKAAKTKD

SEQ ID NO: 552 - protein, 68 aa, Morganella morganii
MPFINIRITKEGATAEQKQKLIEGATQLLVDVLGKNPASTFVIIDEVETDNWGVGGQSVTKLRADAKK

SEQ ID NO: 553 - protein, 78 aa, Mycobacterium sp. URHB0044; identical to SEQ ID NO: 112

SEQ ID NO: 554 - protein, 71 aa, Mycobacterium tusciae; identical to SEQ ID NO: 113

SEQ ID NO: 555 - protein, 62 aa, Neisseria meningitidis 2004032; identical to SEQ ID NO: 114

SEQ ID NO: 556 - protein, 70 aa, Nocardia asteroides NBRC 15531
MPYVNIKVTDEGVTPEQKGQLIKGVTDLLFDVLGKAPASTYVVIDEVPLTDWGVGGVNVAEYRAQRAAEG

SEQ ID NO: 557 - protein, 63 aa, Paenibacillus sp. PAMC 26794; identical to SEQ ID NO: 115

SEQ ID NO: 558 - protein, 78 aa, Paenibacillus durus
MPIVTIQVTREGTAPDRQSVTAEEKAALIKGASELLLNVLNKPLDSTYVIIEEVEPENWGWGGLPALEYRRQRAAKTT

SEQ ID NO: 559 - protein, 67 aa, Paenisporosarcina sp. TG20
MPYVNIKITNEHVTPEKKAKLIEGVTQLLVDVLDKNPETTVVVIDEVDTDNWGIGGRTVTERRGTTK

SEQ ID NO: 560 - protein, 69 aa, Pandoraea sp. B-6
MPFVSIRITREGNTAEQKAQVIREVTETLERVLHKPPHLTHIVIEEIDTDNWGYAGQTTTVVRRQSSQG

SEQ ID NO: 561 - protein, 61 aa, Pantoea sp. A4
MPEVVVFAAEGRSDAQKRTLLKKITEAVVESFGVDKESVIVSIQETPKKHKSRGGVPFDER

SEQ ID NO: 562 - protein, 67 aa, Pantoea sp. A4
MPMIRVEMLTGRTEAQRIELVEALTRETARIAKCPAADIQVIVSEVERTHWAVGGVLNKVSSESNVV

SEQ ID NO: 563 - protein, 61 aa, Pantoea sp. A4
MPEVVVHMASGRTREQKEQLMQKLYLAVKESLSVADEYIVVSLVETPKENKSRGGVPFTAL

SEQ ID NO: 564 - protein, 73 aa, Pelobacter seleniigenes; identical to SEQ ID NO: 116

SEQ ID NO: 565 - protein, 77 aa, Phaseolibacter flectens; identical to SEQ ID NO: 117

SEQ ID NO: 566 - protein, 63 aa, Polaromonas glacialis
MPTYHVEMLEGRTPEQKKKLVEAITRVSVEILGGQPDSVDVLITDIQPGNWATGGKLWSEPRN

SEQ ID NO: 567 - protein, 68 aa, Ponticaulis koreensis
MPFVNIKITKDGATAEQKATLIRGVTDLLVDTLDKNPATTVVVIEEVETDNWGIGGLSVTERRKHPGA

SEQ ID NO: 568 - protein, 69 aa, Ponticaulis koreensis; identical to SEQ ID NO: 38

SEQ ID NO: 569 - protein, 68 aa, Pseudoalteromonas ruthenica
MPYVNIKVTQEGVTQAQKSALIKGVTELLVEVLNKSPATTMVVIDEVELDSWGIGGLPVEQYRRAALE

SEQ ID NO: 570 - protein, 74 aa, Pseudoduganella violaceinigra
MPYVNIRVTKEGVTAEQKLELIKGATELLQRVLNKNPATTFVIIDEVETDNWGIGYNNVTTLRANAAAAKADQR

SEQ ID NO: 571 - protein, 71 aa, Pseudomonas aeruginosa; identical to SEQ ID NO: 16

SEQ ID NO: 572 - protein, 65 aa, Pseudomonas extremaustralis
MPIITINLLEGRTDEQKERLIHEVTEACHRAIDAPRDSVRIIITDMCKQHYGVGGISKLKSQSQQ

SEQ ID NO: 573 - protein, 64 aa, Pseudomonas tolaasii
MPIVKIELFAGRSDEQKARLAKAITQQFVEQLASRPEEVTVLFQDVPPTDWFVAGCSLEKPVVD

SEQ ID NO: 574 - protein, 77 aa, Pseudomonas agarici; identical to SEQ ID NO: 118

SEQ ID NO: 575 - protein, 71 aa, Pseudomonas sp. PH1b
MPYVKIQVTREGVTAEHKRALIEGATELLQRVLNKPPASTFVVIEEIPTDNWGVGGETVTALRQRERDGQG

SEQ ID NO: 576 - protein, 61 aa, Pseudomonas sp. P482
MTDEGVTREQKQQLIEQTTEMLERVLNKPPASTFVVIEEVNTDNWGVGGETVTTLRKREKG

SEQ ID NO: 577 - protein, 62 aa, Ralstonia sp. AU12-08
MPTFHIEMFEGRTVEQKKKLVEEVTRVTCETLGCVPGAVDIIINDVKRENWATGGVLWSEQK

SEQ ID NO: 578 - protein, 75 aa, Rhizobium sp. AP16; identical to SEQ ID NO: 119

SEQ ID NO: 579 - protein, 78 aa, Rhizobium sp. 42MFCr.1
MPIVNVQVTREGTAPGRQSVTEEEKARLIAGVSQVLLEVLNKPLESTYVVIEEVDLDNWGWGGLPTAEYRKLAAAKGT

SEQ ID NO: 580 - protein, 71 aa, Rhodanobacter denitrificans
MPFLNVKLIEGVFTQAQKQEMIRKLTDTMVSIEGENMRPVTWVVIEEVNSGDWGIGGNPLTTSDVKALAKG

SEQ ID NO: 581 - protein, 74 aa, Rudaea cellulosilytica
MPYVNIRITREGATEAQKAELIQGATELLQRVLGKNPKTTIVVIDEVPMENWGIAGLPAEEFRKQNPGSAPKSG

SEQ ID NO: 582 - protein, 63 aa, Saccharopolyspora spinosa
MPFIDVKIFEERLAPETERALISALTDAVAGVLGEKTRPNTWVVLDGTPAQRWGIGGEPGEAK

SEQ ID NO: 583 - protein, 69 aa, Salsuginibacillus kocurii
MPYVNIKVTKENGGLSKEEKQKLIEGSTRLLQETLGKNPNTTVVTIDEIDMDNWGIGAEQISDRRKKGQ

SEQ ID NO: 584 - protein, 76 aa, Schlesneria paludicola
MPIVTIQITREGTTPDQKAALIKGATNLLADVLNKLPSLTFVIIQEVEMEDWGVNGLPAAEHRRLIAAGSLDMPTH

SEQ ID NO: 585 - protein, 77 aa, Serratia sp. M24T3; identical to SEQ ID NO: 120

SEQ ID NO: 586 - protein, 61 aa, Serratia sp. M24T3; identical to SEQ ID NO: 65

SEQ ID NO: 587 - protein, 76 aa, Serratia fonticola; identical to SEQ ID NO: 121

SEQ ID NO: 588 - protein, 74 aa, Serratia sp. S4; identical to SEQ ID NO: 122

SEQ ID NO: 589 - protein, 76 aa, Serratia marcescens; identical to SEQ ID NO: 123

SEQ ID NO: 590 - protein, 69 aa, Serratia liquefaciens FK01
MPYVNIKVTDEAVSAEHKRLLIEGVSKLLEQVLNKPRHTTFVVIDEVATDNWGVGGQSVTELRKSQLQQ

SEQ ID NO: 591 - protein, 74 aa, Shimwellia blattae
MPHIDVKYFPREVSSEEKEAIAAELCAVIQKHFQSKEGALSVTMGTVAPDNWKADVYDRIIHSPDLVKKPGYQM

SEQ ID NO: 592 - protein, 62 aa, Solirubrobacter soli
MPFANLKVPAGLLSEEQKAELVTAVTDLYAVVFGVEARANTMVLVDEVADGGWGIGGTVLTF

SEQ ID NO: 593 - protein, 68 aa, Sphaerotilus natans subsp. natans DSM 6575
MPVVRVTWFDGKDARAKKAVAADITESIQRHTGTDPKWVYVIFEDVAASDWAGSGRLFKEEADDVQPS

SEQ ID NO: 594 - protein, 80 aa, Sphingomonas sp. Mn802worker
MPFVSIRLAGSATRDQKAGIVADVTSSLVTRLGKNPAAVQVVIEEVSPENYGAGGQLIADRDSPATPTTAKGDAHAEPRP

SEQ ID NO: 595 - protein, 76 aa, Sphingomonas sp. PAMC 26617
MPFVSIRLSGTATRDQKSGIVADVTRSLVERLGKNPAAVQIVIEEVSPENYGAGGILIADRDAPKLQEDAHAESSQ

SEQ ID NO: 596 - protein, 63 aa, Sphingopyxis macrogoltabida
MPYINVQFTKGATKEAKREIVKGVTDLMVEHLGKRPEHVHVVIQEVEFEDWGFAGKLVAELSD

SEQ ID NO: 597 - protein, 61 aa, Sporosarcina newyorkensis
MPFVTVKMFEGRTDEQKRALCEKVTEAVAETTGAPSENVVVYIEEMPKNHMSKGGVRASDQ

SEQ ID NO: 598 - protein, 61 aa, Sporosarcina ureae
MPYVTVKMLEGRTDDQKRALCEKVTQAVVETTGATTDSVTVFIEEMPKNHLFKEGKLMSDQ

SEQ ID NO: 599 - protein, 61 aa, Streptacidiphilus jeojiense
MPLIQVKQLTGRTDEQKRALVRELTDAYVRVMGNKPESVWVTIDELPAENWATGGTLLSDR

SEQ ID NO: 600 - protein, 74 aa, Streptococcus pneumoniae
MVKWKKSKLVEEAIMPFVRIDLFEGRTLEQKKALAKEVTEAVVRNTGAPQSAVHVIINDMPEGTYFPQGEMRTK

SEQ ID NO: 601 - protein, 61 aa, Streptococcus pseudoporcinus
MPFITIDLFEGRSQEQKNELAREVTEVVARIAQAPKGNIHVFINDLPEGTYYPQGEMKRKS

SEQ ID NO: 602 - protein, 78 aa, Streptococcus parauberis
MPFVTIDLFEGRTQEQKIQLAREVTDVVSRIAKAPKENIHIMINDMPEGTYFPAGEMKLKTKIFGGFYAPFIYSGHLN

SEQ ID NO: 603 - protein, 61 aa, Streptococcus minor
MPFVTIELFEGRTDDQKTELAREVTEVVSRVAKAPKEAIHVFIHDLPEGTYFPAGEEKKKN

SEQ ID NO: 604 - protein, 61 aa, Streptococcus urinalis
MPFVHIDLFEGRTQEQKNQIAKEVTEVISRVAEAPKENIHVIINDLKEGTYFPQGEMKKKS

SEQ ID NO: 605 - protein, 60 aa, Streptomyces roseoverticillatus; identical to SEQ ID NO: 124

SEQ ID NO: 606 - protein, 66 aa, Streptomyces achromogenes
MPLIRVTLLEGRSPQEVAALGEALTAAAHETLGTPVEAVRVIVEETPPERWFVGGRSVAERRASPS

SEQ ID NO: 607 - protein, 72 aa, Streptomyces pyridomyceticus
MPFIEVKIFEQRLTEQTERELVERLTQATVDVFGESIREQTWVVLTPVPAHRWGIGGSTHPRPAAQEQEPRS

SEQ ID NO: 608 - protein, 68 aa, Streptomyces sp. HmicA12
MPIVTVQQGPRDVEAKRELVKKITDAFVDAYKLPAETVQVWIHEFPADSWGAAGTLTADKQGQKQGQK

SEQ ID NO: 609 - protein, 60 aa, Streptomyces rimosus subsp. rimosus ATCC 10970
MPVVTVQQGPRSTELKRELVRQITDAFVDALHVPAESVQVWIHEVPTDSWGSAGKLTADK

SEQ ID NO: 610 - protein, 68 aa, Sulfitobacter mediterraneus
MPYVNIKVTREGVTPDQKAELISGVTDLLVRVLDKSPATTFVVIDEVALEDWGIGGLPVEQYRNALAR

SEQ ID NO: 611 - protein, 72 aa, Sulfurospirillum arcachonense
MPYVNIKVTNEGVTSEQKQQLIEGATKLLQDVLGKNPATTFVIIEEVETDNWGIAGEQVTKIRKRKENEKDI

SEQ ID NO: 612 - protein, 77 aa, Tatumella saanichensis
MPHIDIKAFPRELTEQQKSELAAEMTQLMQKYLQSREGSVSVALSFVPQEQWKTTVWETEIAPQLETLIKQPDYRMS

SEQ ID NO: 613 - protein, 61 aa, Tatumella saanichensis
MPEVVVFAVEGRSSEQKKKLMKDITDAVVSNFKVDPEVVVVSVVETPKQHKSRGGIPFDEQ

SEQ ID NO: 614 - protein, 64 aa, Thalassobacillus devorans
MPFIQVNILEGRAPATKERFIREVTDLATEVFDAPEQSVRVMINELPPEHWGIAGESVKKRREK

SEQ ID NO: 615 - protein, 64 aa, Thalassobium sp. R2A62
MPIIRVEMLSGRSADQKKALVRELTEAFVKTAGGNPDAIHVVISEVEPSDWAAGGTLYSDKTGH

SEQ ID NO: 616 - protein, 73 aa, Thalassospira lucentensis
MPYVNIKVTREGGPDGTGPTPEEKTQLIKGVTDLLQSVLNKSPATTFVVIDEVSLDDWGISGRSVRDIRATES

SEQ ID NO: 617 - protein, 63 aa, Thauera terpenica
MPFAQIHILEGRTEEQKRAVIEKVTQALIEALDAPQQAVRVLISEVPKTNWGIGGVSAKDLGR

SEQ ID NO: 618 - protein, 64 aa, Thermus igniterrae; identical to SEQ ID NO: 125

SEQ ID NO: 619 - protein, 59 aa, unclassified Cloacimonetes; identical to SEQ ID NO: 127

SEQ ID NO: 620 - protein, 64 aa, uncultured bacterium
MPFVNIKITGKATVAQKNEVIKQVTQTLETVLGKDPKLTHIVIDEVALENWGNCGISAADKEKK

SEQ ID NO: 621 - protein, 75 aa, uncultured microorganism
MPIVNVKLIEGVFSPRQKRDLIEKLTDAVIQVEGENLRSVTWVYIEEVNQGELAIGGRPLSASDVHAMQRGEVAA

SEQ ID NO: 622 - protein, 61 aa, Ureibacillus thermosphaericus
MPVVTIKMLEGRTDEQKKAMVEKVTEALVETINAKPEAVTIIIEEMSKNHYATGGVRASDK

SEQ ID NO: 623 - protein, 78 aa, Variovorax paradoxus
MPFVSIRITREGNTAEQKAQVIREVTDTLERVLHKPPEWTHIVIEEVDTDNWGFGGHTTTAVRQGLAAVPGSRAKTDG

SEQ ID NO: 624 - protein, 64 aa, Vibrio coralliilyticus; identical to SEQ ID NO: 58

SEQ ID NO: 625 - protein, 75 aa, Woodsholea maritima
MPYVNIQVTKEGGPDGTGPSKEEKAALIKGVTGVLHDVLGKDPAKTFVVIEEVDLDNWGCGGLQVREYRALKAKG

SEQ ID NO: 626 - protein, 70 aa, Xylella fastidiosa 32
MPHIIVKIAEGRSQPLKQELADRLAATMMDVLGLDSSAVSVAVEDVPMQDCRPKSQPHRTGHHELRRADR

SEQ ID NO: 627 - protein, 75 aa, Yokenella regensburgei
MPHIDIKCFPRDLNDEQKEALAAEITEVIVRHLNSKDRSVSIALNQVQEADWQQVWEREIAPQMETLIKKPGYSM

(19) United States
(12) Patent Application Publication
(10) Pub. No.: US 2019/0255167 A1
(43) Pub. Date: Aug. 22, 2019

(54) FUSION POLYPEPTIDES

(71) Applicant: Oxford University Innovation Limited, Oxford (GB)

(72) Inventors: David WYLLIE, Oxfordshire (GB); Pauline VAN DIEMEN, Oxfordshire (GB); Christine ROLLIER, Oxfordshire (GB)

(21) Appl. No.: 16/347,209

(22) PCT Filed: Nov. 2, 2017
(86) PCT No.: PCT/GB2017/053300

§ 371 Date: May 3, 2019

(30) Foreign Application Priority Data
Nov. 3, 2016 (GB) 1618536.5

(51) Int. Cl. A61K 39/085; C12N 15/62; C07K 14/31; C12N 15/86; C12N 9/90

(52) U.S. Cl. CPC A61K 39/085; C12N 15/62; C07K 14/31; A61K 2039/64; C12N 9/90; C12Y 503/02006; C12N 15/86

(57) ABSTRACT

The present invention relates to fusion polypeptides comprising (a) a 4-oxalocrotonate tautomerase (4-OT)-based polypeptide scaffold which is capable of forming multimers, and (b) a polypeptide antigen; and homo- and hetero-multimers thereof. The invention also provides nucleic acid molecules and vectors encoding the fusion polypeptides and multimers; and methods of using the fusion polypeptides, multimers, nucleic acid molecules and vectors to produce an immunogenic response against the polypeptide antigen.

The present invention relates to fusion polypeptides comprising (a) a 4-oxalocrotonate tautomerase (4-OT)-based polypeptide scaffold which is capable of forming multimers, and (b) a polypeptide antigen; and homo- and hetero-multimers thereof.

The invention also provides nucleic acid molecules and vectors encoding the fusion polypeptides and multimers; and methods of using the fusion polypeptides, multimers, nucleic acid molecules and vectors to produce an immunogenic response against the polypeptide antigen.

Modern vaccinology approaches use highly-purified protein antigens which often have limited innate stimulatory activity and so may be poorly immunogenic. To address this, multiple strategies have been developed, including incorporating protein antigens into oil emulsions, formulating with aluminium salts, and addition of, or fusion to, Toll-like receptor agonists [1]. Alternatively, by using DNA or recombinant viral vectors, innate immune stimulation can be achieved by the vector while exploiting the host's translational machinery to provide in vivo expression of the antigen [2].

A separate strategy, which has a good safety profile, involves alteration of the size and multimerisation of the antigen either by attachment of the protein to a self-assembling bacteriophage or by fusion to part of a viral capsid [3, 4, 5] or other similar protein [6]. Such virus-like particles (VLPs), which are typically 20-200 nm in diameter, include commercially-deployed human papilloma virus and hepatitis B vaccines [4] as well as numerous products at earlier stages in development [5, 7]. The varied immunological mechanism(s) behind VLP-induced enhanced immunogenicity include ready access to the lymphatic system, rapid dendritic cell uptake and activation, and arrayed-antigen mediated B-cell receptor cross-linking [4]. In some cases, particle entry into cells mediated by specific host receptors has been demonstrated [8, 9]. VLP immunogenicity is likely a result of a combination of these mechanisms.

A limited number of non-viral antigen multimerisation domains have been described. One such domain, IMX313, is derived from the multimerisation domain of a vertebrate complement C4 binding protein (C4 bp) [2], and extensively re-engineered to minimise cross-reactivity with human C4 bp. Marked improvements in immunogenicity to some antigens have been observed with this strategy [10, 11]. Other related technologies include fusion to ferritin or encapsulin molecules [12] or fusion with the highly-multimerising protein lumazine synthetase [13].

Vaccine development is continuing for a range of bacterial pathogens, including S. aureus, pathogenic Neisseria species [14], M. tuberculosis [15], E. coli [14] and against Apicomplexa (e.g. P. falciparum [10]). Multi-antigen vaccines are under development, and there are presently far more candidate antigens than antigen scaffolding strategies. This is potentially problematic, since prior immunity to a scaffold may inhibit immune responses to the antigen-scaffold combination, as was observed with circumsporozoite protein-hepatitis B surface antigen fusions in human adults [16]. It is at present unclear how many molecules exist biologically which are capable of enhancing immunogenicity when fused to other antigens, what the required biophysical properties are, and whether multimerisation is necessary for the adjuvanting effect. Nevertheless, if such proteins exist, pro-immunogenic domains unrelated both to mammalian proteins and to existing viral-like particle components, including Hepatitis B surface antigen, might have utility in a range of vaccines which are currently being developed.

A range of S. aureus proteins which were previously reported to be capable of multimerising (Dps, QacR, SA1388) have now been tested for pro-immunogenic activity using a DNA vaccination system. None displayed a pro-immunogenic effect in this system. This is surprising given the structural similarities between Dps and ferritin, a self-multimerising molecule which is successful at increasing immunogenicity to some antigens when fused to their C-terminus [12].

One S. aureus protein (SAR1376), however, was found to adopt a pro-immunogenic, multimeric, structure in vivo when produced by a DNA vaccination system. This protein is a 4-oxalocrotonate tautomerase (4-OT).

4-Oxalocrotonate tautomerases (4-OTs) are typically 60-80 amino acids in length, placing them among the smallest enzymes known. They have an unusual mechanism of action, involving the proline at residue 1 (after the initiator methionine) and are involved in the catalytic breakdown of polycyclic compounds into tri-carboxylic acid (Krebs') cycle precursors in a variety of bacteria [21]. A range of other enzymatic activities have been described in proteins with 4-OT-like structures [21, 26], but all depend on the initial proline.

SAR1376 was found to be pro-immunogenic in mice when fused to a range of pathogen antigens from S. aureus and from P. falciparum, whether delivered by DNA vaccination, viral vectored vaccines or as protein-in-adjuvant formulations. It is also demonstrated herein by mutagenesis that the adjuvant effect does not depend on enzymatic activity, but is abrogated by mutations unfolding the hexameric structure of the protein.

It is therefore proposed that 4-OT proteins represent a class of pro-immunogenic proteins which can be used as scaffolds and fused to a range of antigens, thus enhancing immune responses against such antigens.

It is an object of the invention, therefore, to provide a fusion polypeptide comprising a polypeptide antigen which is presented on a 4-OT-based polypeptide scaffold. Such fusion polypeptides are capable of forming multimers which can enhance the immunogenicity of the presented antigen.

It is a further object of the invention to provide a nucleic acid molecule, such as a recombinant viral vector, which encodes the fusion polypeptide of the invention, to exploit the host's translational machinery in order to provide in vivo expression of the antigen.

The invention therefore provides a fusion polypeptide, wherein the fusion polypeptide comprises:
(a) a 4-oxalocrotonate tautomerase (4-OT)-based polypeptide scaffold which is capable of forming multimers; and
(b) a polypeptide antigen.

The fusion polypeptide comprises at least two parts.

Part (a) is a 4-oxalocrotonate tautomerase (4-OT)-based polypeptide scaffold which is capable of forming multimers.

The scaffold is based on a 4-oxalocrotonate tautomerase (4-OT) polypeptide. As used herein, the term “4-oxalocrotonate tautomerase (4-OT)-based polypeptide scaffold” means that the scaffold is a 4-oxalocrotonate tautomerase (4-OT) polypeptide or a variant or derivative thereof.

The function of the scaffold is to present the polypeptide antigen in such a manner that the polypeptide antigen elicits an immunogenic response in a subject into which the fusion polypeptide is administered. The scaffold enhances the immunogenicity of the polypeptide antigen. The scaffold may also be said to have an adjuvanting effect on the polypeptide antigen.

The scaffold is capable of multimerising and of presenting the polypeptide antigen in order to elicit an immunogenic response in a subject.

It will be appreciated that the 4-OT polypeptide or a variant or derivative thereof does not have to be enzymatically active in order for it to function as a scaffold.

4-OT-like enzymes are common in bacteria [21]. Using a protein sequence-based search strategy, 2780 discrete family members (with a modal length of 63 amino acids) were found across Eubacteria, with examples in Archaea also. The 2780 sequences were found by using the method described in Example 1.

At least 20% sequence identity was observed in the primary protein sequences between the most diverse members of the family (see FIG. 4), and many members exhibited significantly higher levels of sequence identity.

Furthermore, examination of eleven bacterial crystal structures of 4-OT enzymes showed very similar crystal structures in the family members, despite huge evolutionary distances (see FIG. 4B).

Additionally, conserved motifs, including a highly-conserved initial proline, exist within the primary sequences of 4-OT enzymes from genera known to be pathogenic to man (see FIG. 4C).

The sequences of 26 preferred 4-OT enzymes are given herein as SEQ ID NOs: 1-26.

4-OT enzymes may be tested for by their capability to convert 2-hydroxymuconate to the αβ-unsaturated ketone, 2-oxo-3-hexenedioate. As mentioned above, however, the scaffold polypeptide of the invention does not necessarily have to have such activity; but the presence of such activity may be used to identify 4-OT enzymes from which variants and/or derivatives may be produced.

Taken together, the above information may be used by the skilled artisan to readily identify 4-OT polypeptide and variants and derivatives thereof.

Preferably, the 4-OT polypeptide is from or derived from a bacterium.

More preferably, the 4-OT polypeptide is from or derived from a eubacterium.

Preferably, the 4-OT polypeptide is from of derived from a bacterium of one of the following genera: Acinetobacter, Bacillus, Bartonella, Bordetella, Burkholderia, Campylobacter, Citrobacter, Enterobacter, Escherichia, Haemophilus, Helicobacter, Leptospira, Mycobacterium, Neisseria, Pseudomonas, Rhodobacter, Salmonella, Staphylococcus, Streptococcus, Thermoanaerobacter, Vibrio and Yersinia.

Preferably, the 4-OT polypeptide is from or derived from one of the following bacteria: Acinetobacter baumannii OIFC021, Bacillus cereus, Bartonella elizabethae Re6043vi, Bordetella sp. FB-8, Burkholderia sp. RPE67, Campylobacter jejuni, Citrobacter freundii, Enterobacter cloacae, Escherichia coli, Haemophilus parainfluenzae, Helicobacter pylori, Leptospira interrogans serovar Djasiman str. LT1649, Mycobacterium intracellulare, Neisseria meningitidis, Neisseria gonorrhoeae, Pseudomonas aeruginosa, Pseudomonas putida, Pseudomonas aeruginosa, Rhodobacter sphaeroides, Salmonella enterica, Staphylococcus aureus, Streptococcus pneumoniae, Streptococcus agalactiae, Streptococcus suis, Thermoanaerobacter ethanolicus, Vibrio fluvialis, Yersinia pseudotuberculosis and Yersinia enterocolitica.

In some preferred embodiments, the 4-OT polypeptide is from S. aureus.

More preferably, the 4-OT polypeptide comprises or consists of the amino acid sequence as given in any one of SEQ ID NOs: 1-29 or a variant or derivative thereof.

Most preferably, the 4-OT polypeptide comprises or consists of the amino acid sequence as given in one of SEQ ID NOs: 27-29 or a variant or derivative thereof.

SEQ ID NO: 27 is the wild-type amino acid sequence of the S. aureus 4-OT polypeptide. SEQ ID NO: 28 is the amino acid sequence of the S. aureus 4-OT polypeptide which has a proline→alanine mutation at position 1 (P1A). This mutation disrupts enzymatic activity but does not affect multimerisation. SEQ ID NO: 29 is the amino acid sequence of the S. aureus 4-OT polypeptide which has an arginine→alanine mutation at position 35 (R35A).

In a further embodiment, the invention provides a polypeptide comprising or consisting of an amino acid sequence of SEQ ID NO: 28 or 29.

In other embodiments, the 4-OT polypeptide comprises or consists of one of the amino acid sequences as given in SEQ ID NOs: 38-627, or a variant or derivative thereof.

In some preferred embodiments, the 4-OT polypeptide comprises or consists of an amino acid sequence as given in any one of SEQ ID NOs: 38-127, or a variant or derivative thereof having at least 50% sequence identity thereto, more preferably at least 60%, 70%, 80%, 90% or 95% sequence identity thereto.

In some preferred embodiments, the 4-OT polypeptide comprises or consists of an amino acid sequence as given in any one of SEQ ID NOs: 128-627, or a variant or derivative thereof having at least 80% sequence similarity thereto, more preferably at least 90% or 95% sequence similarity thereto.

In some preferred embodiments, the 4-OT polypeptide comprises or consists of an amino acid sequence as given in any one of SEQ ID NOs: 128-627, or a variant or derivative thereof having at least 80% sequence identity thereto, more preferably at least 90% or 95% sequence identity thereto.

The scaffold of part (a) is a 4-oxalocrotonate tautomerase (4-OT)-based polypeptide. As used herein, this means that the scaffold is a 4-oxalocrotonate tautomerase (4-OT) polypeptide or a variant or derivative thereof.

Any variants or derivatives of the 4-OT polypeptide need to retain their ability to multimerise and their ability to present the antigen in order to elicit an immunogenic response against the antigen in a subject to whom the fusion polypeptide is administered.

As used herein, the term “variants”, when applied to a polypeptide or amino acid sequence, refers to polypeptides having at least 30%, 40%, 50%, 60%, 70%, 80%, 90%, 95% or 99% amino acid sequence identity to the reference SEQ ID NO.

As used herein, the term “derivatives”, when applied to a polypeptide or amino acid sequence, includes fragments of the reference SEQ ID NO which are at least 70%, 80%, 90%, 95% or 99% of the length of the reference SEQ ID NO.

As used herein, the term “variants”, when applied to a nucleotide sequence, refers to polynucleotides having at least 30%, 40%, 50%, 60%, 70%, 80%, 90%, 95% or 99% nucleotide sequence identity to the reference SEQ ID NO.

As used herein, the term “derivatives”, when applied to a nucleotide sequence, includes fragments of the reference SEQ ID NO which are at least 50%, 60%, 70%, 80%, 90%, 95% or 99% of the length of the reference SEQ ID NO.

The polypeptide variants and derivatives must still retain the essential properties of the scaffold, e.g. being capable of multimerising and of presenting the bacterial or viral antigen in order to elicit an immunogenic response in a subject against that antigen.

Variants and derivatives of the nucleotide sequences must encode polypeptides which retain these essential properties.

The polypeptide variants may comprise one or more amino acid substitutions, deletions or insertions compared to the reference sequence.

Preferably, the polypeptide variants comprise one or more amino acid substitutions compared to the reference sequence, most preferably one or more conservative amino acid substitutions.

As used herein, the term “conservative sequence modifications” is intended to refer to amino acid modifications that do not significantly affect or alter ability of the fusion polypeptide to multimerise and to present the bacterial or viral antigen in order to elicit an immunogenic response in a subject against that antigen.

Conservative amino acid substitutions are ones in which the amino acid residue is replaced with an amino acid residue having a similar side chain. Families of amino acid residues having similar side chains have been defined in the art. These families include amino acids with basic side chains (e.g. lysine, arginine, histidine), acidic side chains (e.g. aspartic acid, glutamic acid), uncharged polar side chains (e.g. glycine, asparagine, glutamine, serine, threonine, tyrosine, cysteine, tryptophan), nonpolar side chains (e.g. alanine, valine, leucine, isoleucine, proline, phenylalanine, methionine), beta-branched side chains (e.g. threonine, valine, isoleucine) and aromatic side chains (e.g. tyrosine, phenylalanine, tryptophan, histidine). Thus, one or more amino acids of the scaffold polypeptides may be replaced with another amino acid from the same side chain family, and the modified amino acid sequence may be tested to evaluate its ability to multimerise and to present the bacterial or viral antigen in order to elicit an immunogenic response in a subject.

In some embodiments, the 4-OT polypeptide-based scaffold comprises 1-20, 1-10 or 1-5 or 1-2 conservative amino acid substitutions compared to a 4-OT polypeptide SEQ ID NO disclosed herein.

Percentage amino acid sequence identities and nucleotide sequence identities may be obtained using the BLAST methods of alignment (Altschul et al. (1997), “Gapped BLAST and PSI-BLAST: a new generation of protein database search programs”, Nucleic Acids Res. 25:3389-3402; and http://www.ncbi.nlm.nih.gov/BLAST). Preferably the standard or default alignment parameters are used.

Standard protein-protein BLAST (blastp) may be used for finding similar sequences in protein databases. Like other BLAST programs, blastp is designed to find local regions of similarity. When sequence similarity spans the whole sequence, blastp will also report a global alignment, which is the preferred result for protein identification purposes. Preferably the standard or default alignment parameters are used. In some instances, the “low complexity filter” may be taken off.

BLAST protein searches may also be performed with the BLASTX program, score=50, wordlength=3. To obtain gapped alignments for comparison purposes, Gapped BLAST (in BLAST 2.0) can be utilized as described in Altschul et al. (1997) Nucleic Acids Res. 25: 3389. Alternatively, PSI-BLAST (in BLAST 2.0) can be used to perform an iterated search that detects distant relationships between molecules (see Altschul et al. (1997) supra). When utilizing BLAST, Gapped BLAST, PSI-BLAST, the default parameters of the respective programs may be used.

With regard to nucleotide sequence comparisons, MEGABLAST, discontiguous-megablast, and blastn may be used to accomplish this goal. Preferably the standard or default alignment parameters are used. MEGABLAST is specifically designed to efficiently find long alignments between very similar sequences. Discontiguous MEGABLAST may be used to find nucleotide sequences which are similar, but not identical, to the nucleic acids of the invention.

The BLAST nucleotide algorithm finds similar sequences by breaking the query into short subsequences called words. The program identifies the exact matches to the query words first (word hits). The BLAST program then extends these word hits in multiple steps to generate the final gapped alignments. In some embodiments, the BLAST nucleotide searches can be performed with the BLASTN program, score=100, wordlength=12.

One of the important parameters governing the sensitivity of BLAST searches is the word size. The most important reason that blastn is more sensitive than MEGABLAST is that it uses a shorter default word size (11). Because of this, blastn is better than

MEGABLAST at finding alignments to related nucleotide sequences from other organisms. The word size is adjustable in blastn and can be reduced from the default value to a minimum of 7 to increase search sensitivity.

A more sensitive search can be achieved by using the newly-introduced discontiguous megablast page (www.ncbi.nlm.nih.gov/Web/Newsltr/FallWinter02/blastlab.html). This page uses an algorithm which is similar to that reported by Ma et al. (Bioinformatics. 2002 March; 18(3): 440-5). Rather than requiring exact word matches as seeds for alignment extension, discontiguous megablast uses non-contiguous word within a longer window of template. In coding mode, the third base wobbling is taken into consideration by focusing on finding matches at the first and second codon positions while ignoring the mismatches in the third position. Searching in discontiguous MEGABLAST using the same word size is more sensitive and efficient than standard blastn using the same word size. Parameters unique for discontiguous megablast are: word size: 11 or 12; template: 16, 18, or 21; template type: coding (0), non-coding (1), or both (2).

In some embodiments, the BLASTP 2.5.0+ algorithm may be used (such as that available from the NCBI) using the default parameters.

In other embodiments, a BLAST Global Alignment program may be used (such as that available from the NCBI) using a Needleman-Wunsch alignment of two protein sequences with the gap costs: Existence 11 and Extension 1.

In some embodiments, the NCBI RefSeq database may be queried using BLASTp and delta-BLAST [14] using default parameters. Alignments may be prepared using the NCBI Cobalt multiple alignment engine [35] with default parameters.

Sequences are most preferably aligned pairwise using the Needleman-Wunsch global sequence alignment algorithm (Needleman, Saul B. & Wunsch, Christian D. (1970), “A general method applicable to the search for similarities in the amino acid sequence of two proteins”, Journal of Molecular Biology. 48 (3): 443-53. doi:10.1016/0022-2836(70)90057-4. PMID 5420325) as implemented in the nwalign 0.3.1 python package https://pypi.python.org/pypi/nwalign. Alignments are made with a gap opening penalty of 10 and an extension penalty of 4 using the BLOSUM62 matrix (Henikoff, J. G. Amino acid substitution matrices from protein blocks. Proc. Natl. Acad. Sci. USA 89, 10915-10919 (1992)) obtained from the National Centres for Biotechnology Information. Preferably, for sequence “identity”, at each position in the alignment, a score of 1 is given if the amino acids are identical; zero is assigned in all other positions, including those with gaps. Preferably, for sequence “similarity”, at each position in the alignment, as score of 1 is given if the amino acids in each position are (i) identical, or (ii) come from the same group, where the groups used are: polar positive {Histidine, Lysine, Arginine}; polar negative: {Aspartic acid, Glutamic acid}; polar neutral: {serine, threonine, asparagine, glutamine}; non-polar aliphatic {alanine, valine, leucine, isoleucine, methionine}; {proline and glycine}. This classification is well accepted by those skilled in the field.

The fusion polypeptide also comprises (b) a polypeptide antigen.

The polypeptide may be a “polypeptide-based” antigen, i.e. the antigen is based on a (poly) amino acid sequence which may or may not be glycosylated.

The polypeptide antigen may, for example, be 1-1000, 1-500, 1-200, 1-100 or 1-70 amino acids in length.

The polypeptide antigen may, for example, be one which is presented on a microbe, parasite or neoplasm, or a variant or derivative thereof.

The polypeptide antigen may, for example, be a viral, bacterial, protozoan, animal, mammalian or human antigen, or a variant or derivative thereof.

In some embodiments, the antigen is from a disease-causing bacteria, disease-causing parasite or disease-causing virus, or a variant or derivative thereof.

For example, the antigen may be from a malaria-causing parasite or an influenza-causing virus.

Preferably, the bacterial or parasite antigen is from or derived from Staphylococcus, pathogenic Neisseria, Mycobacteria, Escherichia or from Apicomplexa (e.g. Plasmodium) or helminths.

More preferably, the bacterial or parasite antigen is from or derived from S. aureus, pathogenic Neisseria species, M. tuberculosis, E. coli or P. falciparum.

The polypeptide antigen may, for example, be an polypeptide selected from the group consisting of ClfA, ClfB, FnBPA, FnBP, SdrC, SdrD, SdrE, SasA, SasB, SasC, SasD, SasX, SasF, SasG/AAp, MntC, IsdA, IsdB, IsdH, FhuD2, EsxA, EsxB, Spa, Coa, vWbp, HIa, HIgA, HIgB, HIgC, LukA, LukB, LukD, LukE, EpiP, Can, Csa1A, Csa1B, Csa1C, Csa1D, CsA2A, Csa3A, Csa3B, CsA3C, Csa3D, Csa3E, Csa3G, Csa3H, Csa31, Csa3J, Csa4A, Csa4B, Csa4c, scn, efb, efbc, or a variant or derivative thereof which maintains the immunogenic potential of the antigen.

In one embodiment, the bacterial antigen is S. aureus BitC, a cell surface lipoprotein [19], with accession number NP_370379.

In another embodiment, the bacterial antigen is the extracellular domain of the S. aureus Clumping factor B precursor [20] (ClfB, with accession YP_001333563).

In another embodiment, the bacterial antigen is the S. aureus alpha toxin or a truncated form thereof (e.g. amino acids 1-75 or tHIa75).

In another embodiment, the bacterial antigen is the P. falciparum protein Pfs25.

Preferably, the polypeptide antigen is not a 4-OT polypeptide, or a variant or derivative thereof.

The fusion polypeptide comprises at least two parts, i.e. the scaffold and the antigen. The scaffold and antigen may be linked in any suitable way.

More preferably, however, the scaffold and antigen are joined in the orientation {N-terminus}-antigen-scaffold-{C-terminus}. Preferably, the C-terminus of the scaffold is unmodified.

In some embodiments, the amino acid sequences of the scaffold and antigen are contiguous (i.e. with no intervening amino acids).

In other embodiments, the scaffold and antigen are joined by a linker molecule.

The linker may, for example, be a peptide linker comprising e.g. 1-20 amino acids, or a non-peptide linker.

If present, the linker should not significantly affect (i.e. significantly reduce) the ability of the fusion polypeptide to elicit protective immunity in a subject (e.g. a human subject).

A short amino acid linker may be placed between the antigen and the scaffold sequences. For example, a linker consisting of 1-20, 1-10, 1-5 or 1-3 amino acids may be used.

Preferred linkers include GSG, SGS and SGSG (SEQ ID NO: 36). Most preferably, the linker is GSG. Examples of non-peptide linkers include —(CH₂)ₙ—, wherein n is 1-10.

The fusion polypeptide may comprise a leader sequence. The leader sequence is preferably the human tissue plasminogen activator (tPA) signal sequence.

In some embodiments, the fusion polypeptide may additionally comprise an epitope tag, e.g. the V5 epitope tag (-GKPIPNPLLGLDST-, SEQ ID NO: 37). This may be used to monitor protein expression.

The 4-oxalocrotonate tautomerase (4-OT)-based polypeptide scaffold is capable of forming multimers.

As used herein, the term “capable of forming multimers” means that the scaffold polypeptide is capable of forming n-mers (e.g. dimers, trimers), wherein n=2, 3, 4, 5, 6, 7, 8, 9, 10, 12, 16 or 20, or more.

The invention also provides a multimer formed of two or more fusion polypeptides of the invention. The multimer may be a homo-multimer or hetero-multimer. Preferably, the multimer is a homo-multimer, wherein the fusion polypeptides are all the same.

In one preferred embodiment, the fusion polypeptide is capable of forming hexamers, most preferably homo-hexamers.

In a particularly-preferred embodiment, the fusion polypeptide comprises a 4-OT polypeptide which is capable of forming homo-hexamers.

In other embodiments, the multimer is a hetero-multimer, wherein the fusion polypeptides are not all the same.

In particular, the invention provides a hetero-multimer comprising two or more (e.g. 2, 3, 4, 5, or 6) different fusion polypeptides of the invention.

For example, the fusion polypeptides in the hetero-multimer may comprise the same polypeptide scaffold but different polypeptide antigens. In this way, immunity against a number of different antigens may be achieved using a single hetero-multimer.

For example, the hetero-multimer may comprise two or more different fusion polypeptides of the invention, wherein the polypeptide antigens are derived from different S. aureus antigens.

The fusion polypeptides of the invention may be produced using recombinant methodology. For example, such techniques are described in “Molecular Cloning: A Laboratory Manual” (Fourth Edition), by Michael R. Green and Joseph Sambrook.

Alternatively, the nucleotide sequence encoding the polypeptides may be produced by chemical synthesis. Such a nucleotide sequence may then be ligated into an appropriate vector for host cell transformation or transfection. The polypeptides may then be expressed in such host cells.

For modifications of existing 4-OT genes, CRISPR-based techniques may also be used, such as those described in “CRISPR-Cas: A Laboratory Manual” (2016), edited by Jennifer Doudna (University of California, Berkeley) and Prashant Mali (University of California, San Diego). TALENs-based techniques may also be used.

Alternatively, the polypeptides of the invention may be synthesised using standard chemical peptide synthesis techniques. Solid phase synthesis of peptides in which the C-terminal amino acid of the sequence is attached to an insoluble support followed by sequential addition of the remaining amino acids may, for example, be used.

In a further embodiment, the invention provides a nucleic acid molecule which codes for one or more fusion polypeptides of the invention. Preferably, the nucleic acid molecule encodes two or more, preferably 2, 3, 4, 5 or 6 of the fusion polypeptides of the invention.

Preferred nucleotide sequences include those encoding one or more of SEQ ID NOs: 1-29, and nucleotide sequences having at least 80%, 85%, 90%, or 95% sequence identity thereto, encoding polypeptides which are capable of multimerising and of presenting the bacterial or viral antigen in order to elicit an immunogenic response in a subject.

The nucleic acid molecule may encode two or more different fusion polypeptides of the invention which are capable of forming a hetero-multimer of the invention.

In other preferred embodiments, the nucleotide sequence has or comprises the sequence as given in any one of SEQ ID NOs: 30-32, or a nucleotide sequence having at least 80%, 85%, 90%, or 95% sequence identity thereto, encoding a polypeptide which is capable of multimerising and of presenting the bacterial or viral antigen in order to elicit an immunogenic response in a subject.

As used herein, the terms “nucleic acid sequence”, “nucleic acid molecule” and “polynucleotide” are used interchangeably and do not imply any length restriction. These include DNA (including cDNA) and RNA sequences.

The nucleic acid molecules of the present invention include isolated nucleic acid molecules, i.e. molecules that have been removed from their naturally-occurring environment, recombinant or cloned DNA isolates, and chemically-synthesized analogues or analogues which have been synthesized biologically by heterologous systems.

The nucleic acid molecules of the present invention may be prepared by any means known in the art. For example, large amounts of the polynucleotides may be produced by replication in a suitable host cell. The natural or synthetic DNA fragments coding for a desired fragment may be incorporated into recombinant nucleic acid constructs, typically DNA constructs, capable of introduction into and replication in a prokaryotic or eukaryotic cell. Usually the DNA constructs will be suitable for autonomous replication in a unicellular host, such as yeast or bacteria, but may also be intended for introduction to and integration within the genome of a cultured insect, mammalian, plant or other eukaryotic cell lines.

The nucleic acid molecules of the present invention may also be produced by chemical synthesis, e.g. by the phosphoramidite method or the tri-ester method, and may be performed on commercial automated oligonucleotide synthesizers. A double-stranded fragment may be obtained from the single-stranded product of chemical synthesis either by synthesizing the complementary strand and annealing the strand together under appropriate conditions or by adding the complementary strand using DNA polymerase with an appropriate primer sequence.

The original (e.g. wild-type) codons in a nucleic acid molecule may be optimised for expression in a desired cell line, for example, using an online tool such as that available at http://genomes.urv.es/OPTIMIZER/.

In one embodiment of the invention, therefore, the nucleic acid molecule is codon-optimized for expression in a host cell, preferably a human cell.

The invention also provides a vector or plasmid comprising a nucleic acid molecule of the invention. Preferably, the vector is an expression vector.

The vector and/or plasmid may comprise one or more regulatory sequences which are operably linked to the nucleotide sequence which encodes the fusion polypeptide, e.g. one or more enhancer, promoter and/or transcriptional terminator sequences.

Preferably, the promoter is a CMV promoter, most preferably the CMV IE94 promoter.

Preferably, the vector or plasmid includes a nucleotide sequence which encodes a polyA tail.

Examples of such nucleotide sequences include the bovine growth hormone (BGH) polyadenylation sequence.

In a preferred embodiment, the vector or plasmid comprises elements which encode:

(i) a human tPA leader sequence;

(ii) a bacterial or viral antigen;

(iii) a GSG linker;

(iv) a 4-oxalocrotonate tautomerase (4-OT)-based polypeptide scaffold which is capable of forming multimers; and

(v) a BGH polyA tail.

In some embodiments, the vector is viral vector, e.g. a poxvirus vector. In other embodiments, the vector is an adenoviral vector or a Modified Vaccinia Ankara (MVA) viral vector. Preferably, the vector is a non-replicating vector.

Non-replicating poxviruses and adenoviruses represent groups of viruses which may be used as vectors for the delivery of genetic material into a target cell. Viral vectors serve as antigen delivery vehicles and also have the power to activate the innate immune system through binding cell surface molecules that recognise viral elements. A recombinant viral vector can be produced that carries a nucleic acid encoding a given antigen. The viral vector can then be used to deliver the nucleic acid to a target cell, where the encoded antigen is produced by the target cell's own molecular machinery. As “non-self”, the produced antigen generates an immune response in the target subject.

Without wishing to be bound by any one particular theory, the inventors believe that antigen delivery using the vectors of the invention stimulates, amongst other responses, a T-cell response in the subject. Thus, the inventors believe that one way in which the present invention produces an immunogenic response is by stimulating T-cell responses and the cell-mediated immunity system. In addition, humoral (antibody) based protection can also be achieved.

The vector of the invention may be a non-replicating poxvirus vector. As used herein, a non-replicating (or replication-deficient) viral vector is a viral vector which lacks the ability to productively replicate following infection of a target cell. Thus, a non-replicating viral vector cannot produce copies of itself following infection of a target cell. Non-replicating viral vectors may therefore advantageously have an improved safety profile as compared to replication-competent viral vectors.

In one embodiment, the non-replicating poxvirus vector is selected from a Modified Vaccinia virus Ankara (MVA) vector, a NYVAC vaccinia virus vector, a canarypox (ALVAC) vector, and a fowlpox (FPV) vector. MVA and NYVAC are both attenuated derivatives of vaccinia virus. Compared to vaccinia virus, MVA lacks approximately 26 of the approximately 200 open reading frames.

In one embodiment, the non-replicating poxvirus vector is an MVA vector.

The vector of the invention may be an adenovirus vector. In one embodiment, the adenovirus vector is a non-replicating adenovirus vector (wherein non-replicating is defined as above). Adenoviruses can be rendered non-replicating by deletion of the E1 or both the E1 and E3 gene regions. Alternatively, an adenovirus may be rendered non-replicating by alteration of the E1 or of the E1 and E3 gene regions such that said gene regions are rendered non-functional. For example, a non-replicating adenovirus may lack a functional E1 region or may lack functional E1 and E3 gene regions. In this way the adenoviruses are rendered replication-incompetent in most mammalian cell lines and do not replicate in immunised mammals. Most preferably, both E1 and E3 gene region deletions are present in the adenovirus, thus allowing a greater size of transgene to be inserted. This is particularly important to allow larger antigens to be expressed, or when multiple antigens are to be expressed in a single vector, or when a large promoter sequence, such as the CMV promoter, is used. Deletion of the E3 as well as the E1 region is particularly favoured for recombinant Ad5 vectors. Optionally, the E4 region can also be engineered.

In one embodiment, the adenovirus vector is selected from a human adenovirus vector, a simian adenovirus vector, a group B adenovirus vector, a group C adenovirus vector, a group E adenovirus vector, an adenovirus 6 vector, a PanAd3 vector, an adenovirus C3 vector, a ChAdY25 vector, an AdC68 vector, and an Ad5 vector.

The viral vector of the invention, as described above, can be used to deliver a single antigen to a target cell. Advantageously, the viral vector of the invention can also be used to deliver multiple (different) antigens, homo-multimers or hetero-multimers to a target cell.

In one embodiment, the vector of the invention further comprises a nucleic acid sequence encoding an adjuvant (for example, a cholera toxin, an E. coli lethal toxin, or a flagellin).

The nucleic acid sequence encoding a vector (as described above) may be generated by the use of any technique for manipulating and generating recombinant nucleic acid known in the art. In one aspect, the invention provides a method of making a vector (as described above), comprising providing a nucleic acid, wherein the nucleic acid comprises a nucleic acid molecule encoding a vector of the invention; transfecting a host cell with the nucleic acid molecule; culturing the host cell under conditions suitable for the propagation of the vector; and obtaining the vector from the host cell.

As used herein, “transfecting” may mean any non-viral method of introducing nucleic acid molecules into a cell. The nucleic acid molecule may be any nucleic acid molecule suitable for transfecting a host cell. Thus, in one embodiment, the nucleic acid molecule is a plasmid. The host cell may be any cell in which a vector (i.e. a non-replicating poxvirus vector or an adenovirus vector, as described above) may be grown. As used herein, “culturing the host cell under conditions suitable for the propagation of the vector” means using any cell culture conditions and techniques known in the art which are suitable for the chosen host cell, and which enable the vector to be produced in the host cell. As used herein, “obtaining the vector”, means using any technique known in the art that is suitable for separating the vector from the host cell. Thus, the host cells may be lysed to release the vector. The vector may subsequently be isolated and purified using any suitable method or methods known in the art.

The invention also provides a host cell comprising a nucleic acid molecule, vector or plasmid of the invention.

Preferably, the host cell is a eukaryotic host cell. Examples of eukaryotic host cells include yeast and mammalian cells.

The host cell is preferably a cell in which a vector (e.g. a non-replicating poxvirus vector or an adenovirus vector, as described above) may be grown or propagated. The host cell may be selected from a 293 cell (also known as a HEK, or human embryonic kidney, cell), a CHO cell (Chinese Hamster Ovary), a CCL81.1 cell, a Vero cell, a HELA cell, a Per.C6 cell, a BHK cell (Baby Hamster Kidney), a primary CEF cell (Chicken Embryo Fibroblast), a duck embryo fibroblast cell, or a DF-1 cell.

In other embodiments, the host cell is a human cell (e.g. an isolated human cell).

As used herein, the term “product of the invention” refers to the fusion polypeptides of the invention, multimers of the invention, nucleic acids of the invention and vectors and plasmids of the invention, inter alia.

The invention provides a composition comprising a product of the invention.

In particular, the invention provides a composition comprising one or more fusion polypeptides of the invention, one or more multimers of the invention, one or more nucleic acid molecules of the invention, and/or one or more vectors of the invention, optionally together with one or more pharmaceutically-acceptable carriers, excipients or diluents.

The composition is an immunogenic composition.

Substances suitable for use as pharmaceutically-acceptable carriers are known in the art. Non-limiting examples of pharmaceutically-acceptable carriers include water, saline, and phosphate-buffered saline. In some embodiments, however, the composition is in lyophilized form, in which case it may include a stabilizer, such as bovine serum albumin (BSA). In some embodiments, it may be desirable to formulate the composition with a preservative, such as thiomersal or sodium azide, to facilitate long term storage. Examples of buffering agents include, but are not limited to, sodium succinate (pH 6.5), and phosphate buffered saline (PBS; pH 7.4).

In addition to a pharmaceutically-acceptable carrier, the composition of the invention can be further combined with one or more of a salt, excipient, diluent, adjuvant, immunoregulatory agent and/or antimicrobial compound.

In one embodiment, the products of the invention may contain 5% to 95% of active ingredient (i.e. fusion polypeptide, multimer, nucleic acid, vector), such as at least 10% or 25% of active ingredient, or at least 40% of active ingredient or at least 50%, 55%, 60%, 70% or 75% active ingredient.

The products of the invention may be administered in a manner compatible with the dosage formulation, and in such amount as will be prophylactically and/or therapeutically effective.

Administration of the products of the invention is generally by conventional routes, e.g. intravenous, subcutaneous, intraperitoneal, or mucosal routes. The administration may be by parenteral administration; for example, a subcutaneous or intramuscular injection.

Accordingly, the products of the invention may be prepared as injectables, either as liquid solutions or suspensions. Solid forms suitable for solution in, or suspension in, liquid prior to injection may alternatively be prepared. The preparation may also be emulsified, or the peptide encapsulated in liposomes or microcapsules. The active ingredients are often mixed with excipients which are pharmaceutically acceptable and compatible with the active ingredient. Suitable excipients are, for example, water, saline, dextrose, glycerol, ethanol, or the like and combinations thereof. In addition, if desired, the products of the invention may also contain minor amounts of auxiliary substances such as wetting or emulsifying agents, and/or pH buffering agents.

Additional formulations which are suitable for other modes of administration include oral formulations or formulations suitable for distribution as aerosols. Oral formulations include such normally employed excipients as, for example, pharmaceutical grades of mannitol, lactose, starch, magnesium stearate, sodium saccharine, cellulose, magnesium carbonate, and the like. These compositions take the form of solutions, suspensions, tablets, pills, capsules, sustained release formulations or powders.

It may be desired to direct the products of the present invention to the respiratory system of a subject. Efficient transmission of a therapeutic/prophylactic composition or medicament to the site of infection in the lungs may be achieved by oral or intra-nasal administration.

Formulations for intranasal administration may be in the form of nasal droplets or a nasal spray. An intranasal formulation may comprise droplets having approximate diameters in the range of 100-5000 μm, such as 500-4000 μm, 1000-3000 μm or 100-1000 μm. Alternatively, in terms of volume, the droplets may be in the range of about 0.001-100 μl, such as 0.1-50 μl or 1.0-25 μl, or such as 0.001-1 μl.

Alternatively, the composition may be an aerosol formulation. The aerosol formulation may take the form of a powder, suspension or solution. The size of aerosol particles is relevant to the delivery capability of an aerosol. Smaller particles may travel further down the respiratory airway towards the alveoli than would larger particles. In one embodiment, the aerosol particles have a diameter distribution to facilitate delivery along the entire length of the bronchi, bronchioles, and alveoli. Alternatively, the particle size distribution may be selected to target a particular section of the respiratory airway, for example the alveoli. In the case of aerosol delivery of the medicament, the particles may have diameters in the approximate range of 0.1-50 μm, preferably 1-25 μm, more preferably 1-5 μm.

Aerosol particles may be for delivery using a nebulizer (e.g. via the mouth) or nasal spray. An aerosol formulation may optionally contain a propellant and/or surfactant.

Preferably, the composition of the invention is a vaccine composition, e.g. suitable for parenteral administration, optionally together with one or more adjuvants.

As used herein, a vaccine is a formulation that, when administered to an animal subject such as a mammal (e.g. a human, bovine, porcine, ovine, caprine, equine, cervine, canine or feline subject; in particular a human subject), stimulates a protective immune response against an infectious disease. The immune response may be a humoral and/or a cell-mediated immune response. Thus, the vaccine may stimulate B cells and/or T cells.

Examples of suitable adjuvants include those which are selected from the group consisting of:
metal salts such as aluminium hydroxide or aluminium phosphate,
oil in water emulsions,
toll like receptors agonist, (such as toll like receptor 2 agonist, toll like receptor 3 agonist, toll like receptor 4 agonist, toll like receptor 7 agonist, toll like receptor 8 agonist and toll like receptor 9 agonist),
saponins, for example Quil A and its derivatives such as QS7 and/or QS21,
CpG containing oligonucleotides,
3D-MPL,
(2-deoxy-6-o-[2-deoxy-2-[(R)-3-dodecanoyloxytetra-decanoylamino]-4-o-phosphono-β-D-glucopyranosy]]-2-[(R)-3-hydroxytetradecanoylamino]-α-D-glucopyranosyldihydrogenphosphate),
DP (3S, 9 R)-3-[(R)-dodecanoyloxytetradecanoylamino]-4-oxo-5-aza-9(R)-[(R)-3-hydroxytetradecanoylamino] decan-1, 10-diol, 1,10-bis(dihydrogenophosphate), and
MP-Ac DP (3S-,9R)-3-[(R)-dodecanoyloxytetradecanoylamino]-4-oxo-5-aza-9-[(R)-3-hydroxytetradecanoylamino]decand, 10-diol, 1-dihydrogenophosphate 10-(6-aminohexanoate),
   or combinations thereof.

Preferably, the adjuvant is selected from the group comprising:
a saponin associated with a metallic salt, such as aluminium hydroxide or aluminium phosphate
3D-MPL, QS21 and a CpG oligonucleotide, for example as an oil in water formulation,
saponin in the form of a liposome, for example further comprise a sterol such as QS21 and sterol, and
ISCOM.

In some particularly preferred embodiments, the adjuvant comprises a saponin. Saponins are steroid or triterpenoid glycosides, which occur in many plant species. Saponin-based adjuvants act in part by stimulating the entry of antigen-presenting cells into the injection site and enhancing antigen presentation in the local lymph nodes.

Preferably, the adjuvant comprises saponin, cholesterol and a phospholipid, e.g. ISCOM Matrix-M™ (Isconova, Novavax).

In Matrix-M, purified saponin fractions are mixed with synthetic cholesterol and a phospholipid to form stable particles than can be readily formulated with a variety of vaccine antigens. Matrix-M™ induces both a cell-mediated and an antibody mediated immune response.

In some other preferred embodiments, the adjuvant comprises a squalene-oil-in-water nano-emulsion emulsion, e.g. AddaVax™ (InvivoGen).

Squalene is an oil which is more readily metabolized than the paraffin oil used in Freund's adjuvants. Squalene oil-in-water emulsions are known to elicit both cellular (Th1) and humoral (Th2) immune responses. This class of adjuvants is believed to act through recruitment and activation of APC and stimulation of cytokines and chemokines production by macrophages and granulocytes.

The composition may further comprise a surfactant. Examples of suitable surfactants include Tween (such as Tween 20), briji and polyethylene glycol.

Vaccine preparation is generally described in New Trends and Developments in Vaccines, edited by Voller et al., University Park Press, Baltimore, Md., U.S.A., 1978. Encapsulation within liposomes is described, for example, by Fullerton, U.S. Pat. No. 4,235,877.

The amount of the polypeptide, multimer, nucleic acid molecule or vector of the present invention present in each vaccine dose is selected as an amount which induces an immunoprotective response without significant, adverse side effects in typical vaccines. Such amount will vary depending upon which specific immunogen is employed and whether or not the vaccine is adjuvanted. Generally, it is expected that each dose will comprise 1-1000 μg of protein, for example 1-200 μg, such as 10-100 μg, and more particularly 10-40 μg. An optimal amount for a particular vaccine can be ascertained by standard studies involving observation of antibody titres and other responses in subjects. Following an initial vaccination, subjects will preferably receive a boost in about 4 weeks, followed by repeated boosts every six months for as long as a risk of infection exists. The immune response to the products of this invention is enhanced by the use of adjuvant and or an immunostimulant.

The amount of saponin for use in the adjuvants of the present invention may be in the region of 1-1000 μg per dose, generally 1-500 μg per dose, more such as 1-250 μg per dose, and more specifically between 1 to 100 μg per dose (e.g. 10, 20, 30, 40, 50, 60, 70, 80 or 90 μg per dose).

The invention also provides a combined preparation comprising two or more components selected from two or more fusion polypeptides of the invention, two or more multimers of the invention, two or more nucleic acids of the invention, two or more vectors of the invention and two or more compositions of the invention as a combined preparation in a form suitable for simultaneous, separate or sequential use, preferably for use in producing an immunogenic response to the polypeptide antigen in a subject.

In yet another aspect, the invention provides an antibody against a fusion polypeptide of the invention.

In yet further embodiments, the invention provides a fusion polypeptide of the invention, a multimer of the invention, a nucleic acid of the invention, a vector of the invention or a composition of the invention for use in therapy or for use as a medicament.

In a further aspect, the invention provides a fusion polypeptide of the invention, a multimer of the invention, a nucleic acid of the invention, a vector of the invention or a composition of the invention for use in producing an immunogenic response to the polypeptide antigen in a subject.

In a further aspect, the invention provides a fusion polypeptide of the invention, a nucleic acid of the invention, a vector of the invention or a composition of the invention for use in a method of inducing a T-cell or B-cell response to the polypeptide antigen in a subject.

In particular, a non-replicating poxvirus vector of the invention can be used to stimulate a protective immune response via the cell-mediated immune system. In one embodiment, the T-cell is a T-helper cell (Tₕ-cell). In one embodiment, the T-cell is a Tₕ17-cell.

In further embodiments, the invention provides the use of a fusion polypeptide of the invention, a multimer of the invention, a nucleic acid of the invention, a vector of the invention or a composition of the invention in the manufacture of a medicament for use in producing an immunogenic response to the polypeptide antigen in a subject.

In further embodiments, the invention provides the use of a fusion polypeptide of the invention, a multimer of the invention, a nucleic acid of the invention, a vector of the invention or a composition of the invention in the manufacture of a medicament for use in a method of inducing a T cell or B-cell response to the polypeptide antigen in a subject.

The invention also provides a method of producing an immunogenic response to a polypeptide antigen in a subject, the method comprising administering an effective amount of a fusion polypeptide of the invention, a multimer of the invention, a nucleic acid of the invention, a vector of the invention or a composition of the invention to the subject.

The invention also provides a method of inducing a T-cell or B-cell response to a polypeptide antigen in a subject, method comprising administering an effective amount of a fusion polypeptide of the invention, a multimer of the invention, a nucleic acid of the invention, a vector of the invention or a composition of the invention to the subject.

A polypeptide of the invention, a multimer of the invention, a nucleic acid of the invention, a vector of the invention or a composition of the invention may also be used in similar uses and methods to produce neutralising antibodies in vivo against the polypeptide antigens.

The efficacy of the uses and methods to produce an immunogenic response in a subject may be tested (e.g. by ELISA) by establishing the presence or absence of neutralising antibodies against the polypeptide antigen in the subject's blood.

Also provided is an immunogenic composition comprising two or more fusion polypeptides, two or more nucleic acid molecules or two or more vectors or plasmids as defined herein as a combined preparation in a form suitable for simultaneous, separate or sequential use for stimulating an immune response in a subject against the polypeptide antigen.

Preferably, the fusion polypeptide (or other product of the invention) increases the antibody response in a subject (e.g. mouse or human) against the polypeptide antigen (e.g. as measured by a LIPS assay) compared to the antibody response produced by a control polypeptide which encodes the polypeptide antigen alone (i.e. in the absence of the polypeptide scaffold).

Preferably, the fusion polypeptide (or other product of the invention) increases the number or concentration of IFN-γ producing T-cells in a subject (e.g. as measured by IFN-γ ELISpot assay) compared to the number or concentration of IFN-γ producing T-cells produced by a control polypeptide which encodes the polypeptide antigen alone (i.e. in the absence of the polypeptide scaffold).

The subject is preferably a mammal, e.g. a human, pig, cow or horse, more preferably a human.

As used herein, the term “preventing” includes preventing the initiation of a bacterial or viral infection and/or reducing the severity of intensity of a bacterial or viral infection.

Thus, “preventing” encompasses vaccination.

As used herein, the term “treating” embraces therapeutic and preventative/prophylactic measures (including post-exposure prophylaxis) and includes post-infection therapy and amelioration of a bacterial or viral infection. Each of the above-described methods and uses can comprise the step of administering to a subject an effective amount, such as a therapeutically-effective amount, of a fusion polypeptide of the invention, a multimer of the invention, a nucleic acid of the invention, a vector of the invention or a composition of the invention.

As used herein, an effective amount is a dosage or amount that is sufficient to achieve a desired biological outcome. As used herein, a therapeutically-effective amount is an amount which is effective, upon single or multiple dose administration to a subject (such as a mammalian subject, in particular a human subject) for treating, preventing, curing, delaying, reducing the severity of, ameliorating at least one symptom of a disorder or recurring disorder, or prolonging the survival of the subject beyond that expected in the absence of such treatment.

Accordingly, the quantity of active ingredient to be administered depends on the subject to be treated, capacity of the subject's immune system to generate a protective immune response, and the degree of protection required. Precise amounts of active ingredient required to be administered may depend on the judgement of the practitioner and may be particular to each subject. Administration to the subject can comprise administering to the subject a fusion polypeptide of the invention, a multimer of the invention, a nucleic acid of the invention, a vector of the invention or a composition of the invention (i.e. a product of the invention) wherein the product of the invention is sequentially administered multiple times (for example, wherein the composition is administered two, three or four times). Thus, in one embodiment, the subject is administered a fusion polypeptide of the invention, a multimer of the invention, a nucleic acid of the invention, a vector of the invention or a composition of the invention and is then administered the same product of the invention (or a substantially similar product) again at a different time.

In one embodiment, administration to a subject comprises administering a fusion polypeptide of the invention, a multimer of the invention, a nucleic acid of the invention, a vector of the invention or a composition of the invention to a subject, wherein said product of the invention is administered substantially prior to, simultaneously with, or subsequent to, another immunogenic composition.

The invention also extends to prime-boost regimes. For example, priming and/or boosting may be effected using one or more products of the invention. The products may be administered to a subject sequentially, simultaneously or separately. The first and subsequent products of the invention may be the same or different.

The fusion polypeptides may be in the form of a pharmaceutical composition, preferably a vaccine composition, optionally together with one or more pharmaceutically-acceptable carriers, diluents, excipients and adjuvants.

In one embodiment, the first and second products of the invention are administered as part of a prime-boost administration protocol. Thus, the first product may be administered to a subject as the “prime” and the second product subsequently administered to the same subject as the “boost”.

In one preferred embodiment, the first product is an adenovirus vector of the invention prime (e.g. AdHu5), and the second product is a non-replicating poxvirus vector of the invention boost (e.g. MVA).

In one embodiment, each of the above-described methods further comprises the step of administration to the subject of a product of the invention.

In one embodiment, the polypeptide of the invention is administered separately from the administration of a viral vector of the invention. Preferably the fusion polypeptide and a viral vector are administered sequentially, in any order. Thus, in one embodiment, the viral vector (“V”) and the fusion polypeptide (“P”) may be administered in the order V-P, or in the order P-V.

In certain embodiments, the above-described methods further comprise the administration to the subject of an adjuvant. Adjuvant may be administered with any of the products of the invention.

The products of the invention may be given in a single dose schedule (i.e. the full dose is given at substantially one time). Alternatively, the products of the invention may be given in a multiple-dose schedule.

A multiple-dose schedule is one in which a primary course of treatment (e.g. vaccination) may be with 1-6 separate doses, followed by other doses given at subsequent time intervals required to maintain and or reinforce the immune response, for example (for human subjects), at 1-4 months for a second dose, and if needed, a subsequent dose(s) after a further 1-4 months.

The dosage regimen will be determined, at least in part, by the need of the individual and be dependent upon the judgment of the practitioner (e.g. doctor or veterinarian).

Simultaneous administration means administration at (substantially) the same time.

Sequential administration of two or more products of the invention means that the products are administered at (substantially) different times, one after the other.

For example, sequential administration may encompass administration of two or more products of the invention at different times, wherein the different times are separated by a number of days (for example, 1, 2, 5, 10, 15, 20, 30, 60, 90, 100, 150 or 200 days).

For example, in one embodiment, the vaccine of the present invention may be administered as part of a ‘prime-boost’ vaccination regime.

In one embodiment, the products of the invention can be administered to a subject such as a mammal (e.g. a human, bovine, porcine, ovine, caprine, equine, cervine, canine or feline subject) in conjunction with (simultaneously or sequentially) one or more immunoregulatory agents selected from, for example, immunoglobulins, antibiotics, interleukins (e.g. IL-2, IL-12), and/or cytokines (e.g. IFN-γ).

In yet further embodiments, the invention provides a process for the production of one or more of fusion polypeptides of the invention, which process comprises expressing one or more nucleic acid molecules coding for one or more of said fusion polypeptides in a suitable host, and recovering the polypeptide product(s).

Preferably, the polypeptide products are recovered as multimers.

Preferably, the host is a human cell.

The disclosure of each reference set forth herein is specifically incorporated herein by reference in its entirety.

### BRIEF DESCRIPTION OF THE FIGURES

FIG. 1. Crystal structures of four multimerizing domains examined (PDB references are shown beneath each image pair).

FIG. 2. Antibody responses to BitC measured by LIPS after vaccination with different scaffolds fused to the c-terminus of BitC. Panel A) Design of pMono2 DNA vaccination vectors; CMV: CMV IE94 promoter, TPA: human tissue plasminogen activator signal sequence, Antigen: antigen being tested, V5: epitope tag and linker sequence, Scaffold: molecule under test, BGH pA: Bovine growth hormone polyadenylation sequence. Panel B) BitC-specific antibody response after prime-boost immunisations of groups of 4-5 BALB/c mice with 50 μg BitC-scaffold DNA as measured by LIPS assay. Sera were collected 3 weeks post boost. Each symbol represents a single mouse. Dotted line: threshold for background response. *p<0.05.

FIG. 3. SAR1376 scaffold enhances immune responses to BitC and ClfB in BALB/c mice. Antigen specific antibody responses against BitC (panel A) and ClfB (panel C) as measured by LIPS assay, and number of IFN-γ producing T-cells induced by BitC (panel B) and ClfB (panel D) after vaccination with different scaffolds fused to the c-terminus of the antigens. Sera were collected 3 weeks post boost. Each symbol represents a single mouse. Dotted line: threshold for background response. **p<0.01; ***p<0.001.

FIG. 4. 4-OT family members. A) Family tree, 2780 discrete family members (modal length of 63 amino acids) were found across Eubacteria. B) Published crystal structures of 4-OT members from diverse genera, including SAR1376, whose positions are indicated in A. C) Cobalt alignment of twenty-four 4-OT like sequences from selected genera which include human pathogens.

FIG. 5. SAR1376 mutant P1A scaffold enhances immune responses to ClfB in BALB/c and CD1 mice. Antigen specific antibody responses against ClfB in BALB/c (panel A) and CD1 (panel C) mice as measured by LIPS assay, and number of IFN-γ producing T-cells induced by ClfB in BALB/c (panel B) and CD1 (panel D) mice after vaccination with mutant scaffolds fused to the c-terminus of the antigen. Sera were collected 3 weeks post boost. Each solid symbol represents a single mouse. Dotted line: threshold for background response. *p<0.05; **p<0.01.

FIG. 6. SAR1376 specific antibodies measured after vaccination with (mutant) scaffold fused to the c-terminus of the antigen. Panel A) antibody response against SAR1376 in BALB/c mice immunised with BitC-SAR1376 fusion protein; Panel B) anti-SAR1376 antibody levels after vaccination of BALB/c mice with ClfB fused to SAR1376, SAR1376-P1A or SAR1376-R35A, Panel C) anti-SAR1376 antibodies in CD1 mice immunised with ClfB-SAR1376 mutant PIA scaffold, as measured by LIPS assay. Each solid symbol represents a single mouse. ***p<0.001; ****p<0.0001.

FIG. 7. SAR1376 mutant PIA fused to truncated HIa increases (functional) antibody response when expressed in Viral Vectors. Panel A) Anti α-hemolysin antibody levels, Panel B) anti SAR1376 antibody levels after vaccination of BALB/c mice with tHIa75-PIA, tHIa75-No scaffold, or empty vectors (controls) as assessed respectively by ELISA and LIPS assay. Sera were collected pre boost (day 56 post prime), and post boost (day 70 post prime). Panel C) α-Toxin neutralizing activity of the antibodies was assessed in day 70 serum by Neutralisation Assay. Minimum and Maximum level as determined with mAb 8B7 as described in Methods. **p<0.01.

FIG. 8. Fusion of SAR3176-P1A to Pfs25 improves functional activity.

FIG. 8A) Plasmid construct for expression of pfs25-SAR1376 P1A in Pichia pastoris.

FIG. 8B) Antibody response after vaccination of BALB/c mice with Pfs2525 fused to SAR1376-P1A. Sera were collected at day 14, 28 and day 61 post prime and IgG responses measured using a standardised ELISA. Each solid symbol represents a single mouse.

FIG. 8C) Pooled-purified IgG mixed with malaria infected blood was fed to Anopheles stephensi mosquitos through a membrane. Midguts were dissected 9 days post-feed and the number of oocysts per gut were counted. Each solid symbol represents the count from one mosquito. AU=antibody units. *p<0.05.

### EXAMPLES

The present invention is further illustrated by the following Examples, in which parts and percentages are by weight and degrees are Celsius, unless otherwise stated. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions. Thus, various modifications of the invention in addition to those shown and described herein will be apparent to those skilled in the art from the foregoing description. Such modifications are also intended to fall within the scope of the appended claims.

### Example 1: Methods

Scaffolds Used and their Production

Chosen scaffold sequences were human codon optimised and DNA synthetized (GeneArt, Life Technologies Ltd) (SEQ ID NOs: 38-127). Scaffolds were fused to the C-terminus of the S. aureus antigens in a mammalian expression vector (pMono2) using a restriction enzyme based strategy. Antigens fused were S. aureus BitC, a S. aureus cell surface lipoprotein (accession NP_370379); the extracellular domain of the S. aureus Clumping factor B precursor [20] (ClfB, accession YP_001333563); S. aureus α-hemolysin (accession YP_111574996, amino acids 1-75, designed tHIa75 here); and P. falciparum protein Pfs25 (accession AAN35500) [10]. Sequences for all these were synthesised by Life Technologies Ltd.

S. aureus tHIa75 was ligated into the pMono2 vector from which the construct was subcloned into shuttle vectors and transfected into replication-deficient adenovirus human serotype 5 (AdHu5) and Modified Vaccinia Ankara (MVA) as described elsewhere [29, 30].

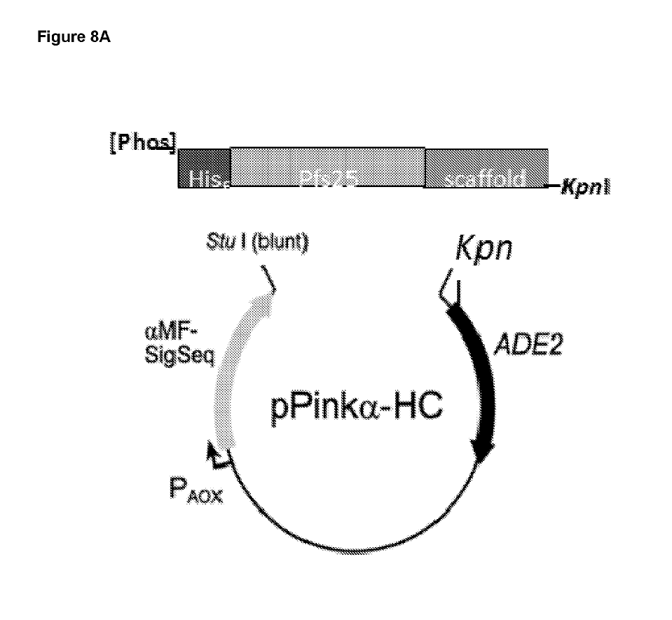

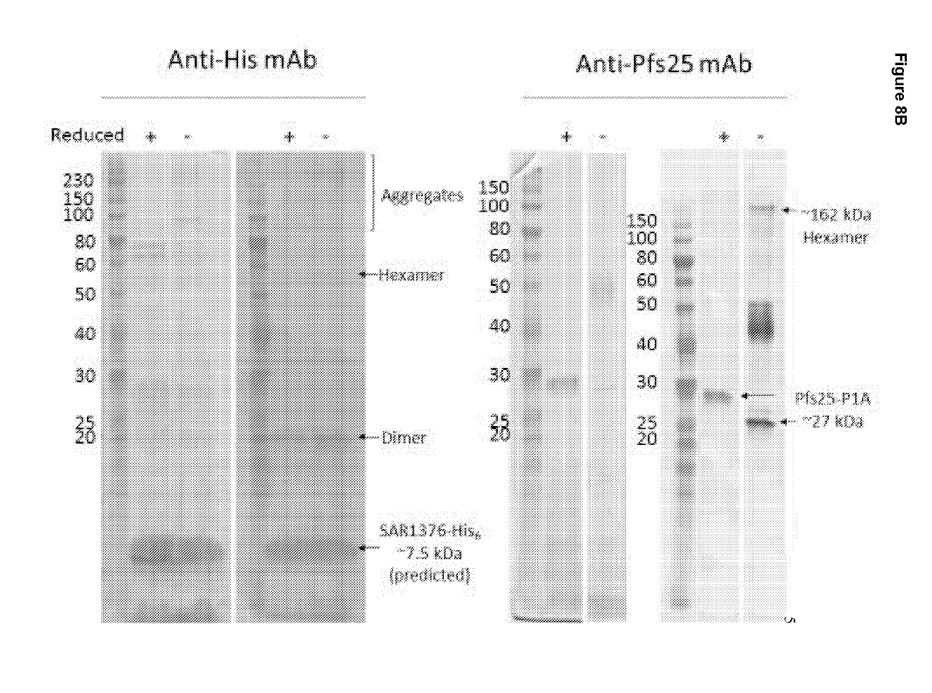

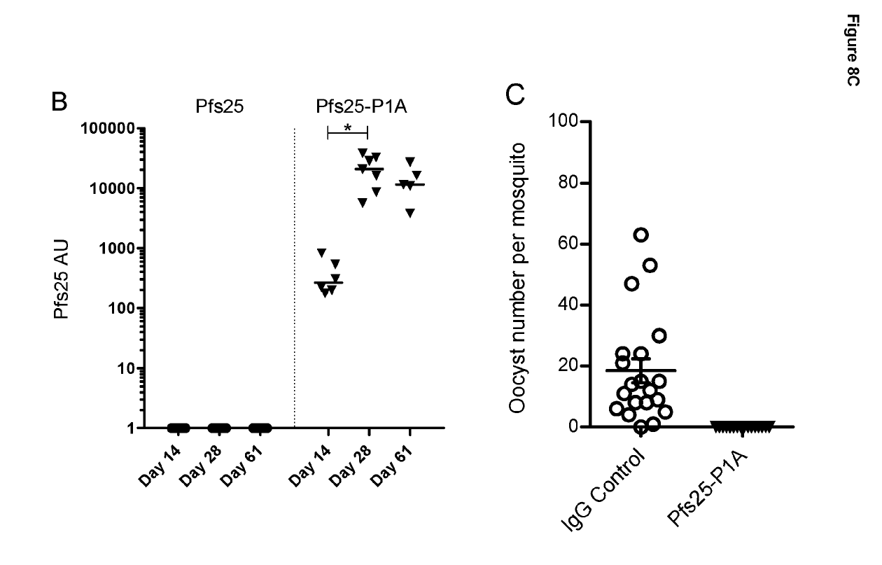

The gene coding for Plasmodium falciparum transmission-blocking antigen, Pfs25, with a 6-his tag (His₆-Pfs25) was fused 5′ of the gene coding for SAR1376-PIA and ligated into the pPinka-HC plasmid (FIG. 8A) which puts protein expression under methanol-inducible control and allows secretion of the expression product from Pichia pastoris via the α-mating factor secretion signal. Electrocompetent P. pastoris were transformed with the expression plasmid. Colonies were screened for optimal expression and the highest expressing clone selected for scale up. A one litre shake flask culture of the selected clone was grown under inducing conditions and the supernatant harvested. Supernatant containing the secreted expression product was harvested by centrifugation and the product purified by nickel-chelate affinity chromatography.

Vaccination Experiments

All mouse procedures were conducted in accordance to the Animal (Scientific Procedures) Act 1986 (Project licence 30/2825) and were approved by the University of Oxford Animal Care and Ethical Review Committee. Six to eight week old female BALB/c or CD1 mice from Harlan Laboratories UK were used.

In DNA vaccination experiments, groups of 4-12 mice (BALB/c or CD1) were immunised intramuscularly with 50 μg vector DNA in 50 μl PBS (25 μl/hind leg). Immunisation was repeated 2 weeks later. On day 35, blood samples were taken from all animals under terminal anaesthesia (heart bleeds) for immune assays (IFN-gamma secreting T-cell numbers (ELISpot), and antibody levels by Luciferase ImmunoPrecipitation System (LIPS) assay) [31-32].

For viral vector immunization, groups of 6 mice were immunised intramuscularly with 10⁹ i.u. AdHu5 in 25 μl PBS followed at least 8 weeks later by 10⁷ pfu MVA as prime-boost sequence, a regime we refer to as AM7. Venous blood samples were taken from the tail vein of all animals pre boost and 2 weeks post boost.

For immunization with recombinant proteins, groups of 6 BALB/c mice were immunised intramuscularly with 50 μl aliquots (25 μl/hind leg) of protein-in-Alhydrogel formulations, containing 2.5 μg of either Pfs25-P1A or monomeric Pfs25 twice at 2 week intervals.

Blood was collected from the tail vein on day 14 (2 weeks post prime) and day 28 (2 weeks post boost).

Assessment of Immune Responses Against BitC and ClfB

A Luciferase ImmunoPrecipitation System (LIPS) assay was used to detect specific serum anti-S. aureus BitC and ClfB antibodies as described [32]. Briefly, recombinant BitC and ClfB fusion protein with Renilla luciferase were produced in 293 cells as described [32]. Serially diluted sera were incubated with Renilla luciferase-BitC or ClfB fusion proteins. The mix was added to filter plates loaded with A/G beads (Thermo Fisher). After incubation and subsequent washings, chemiluminescence was measured in a Luminometer (ClarioStar, BMG Labtech) after adding substrate (Renilla luciferase assay system, Promega UK Ltd.). Log transformation was applied to luminescence data prior to statistical analysis. Specific luminescence was generated by subtracting the assay background, which was considered to be the luminescence observed in the absence of any sera. The assay limit of detection was considered to be four standard deviations above the specific luminescence in the control groups.

Anti-Alpha Toxin Immune Responses

The anti-tHIa antibody levels and functional activity (neutralizing activity, NA) of the antibodies in serum were assessed respectively by ELISA and Toxin Neutralisation Assay (TNA) as described by Oscherwitz and Cease [33]. In brief, the ability of antibody to block recombinant alpha toxin (AT) cytotoxicity in vitro was assessed using the Jurkat T cell line (TIB-152, ATCC, Manassas, Va.). Mouse anti-Staphylococcal alpha hemolysin mAb (8B7) (IBT Bioservices #0210-001) was used as standard positive to obtain minimum and maximum levels for neutralisation of AT (H9395, Sigma Biologicals).

Anti-Pfs25 Immune Responses

Antibody levels in serum were assessed by standardised anti-Pfs25 ELISA, as described [10]. A serially-diluted standard reference serum with a known antibody titre was used to determine the antibody titre of individual samples. Total IgG was purified from the pooled serum of the mice immunised with Pfs25-SAR1376-P1A and assessed by functional assay by Standard Membrane Feeding Assay (SMFA). This assay involves feeding malaria infected blood mixed with purified IgG to Anopheles stephensi mosquitos through a membrane [34]. If the IgG has functional activity, it will block development of the malaria sexual stage in the mosquito midgut; and at 9 days post feed there will be a reduction in the number of oocysts observed in the gut compared to a non-functional IgG control.

Statistical Analysis

Data on antibody response and IFNγ-specific spots were statistically analysed for effect of added scaffold by means of an F-test after a log₁₀ transformation and correction for background. Log(number of IFN-γ secreting cells) was used, because of the approximate log-normal distribution of ELISpot counts in the animals (not shown). Specific antibody levels from the LIPS assay were generated by subtracting the assay luminescence background, which was considered to be the luminescence observed in the absence of any sera, from the luminescence observed with serum dilutions added. The assay limit of detection was considered to be four standard deviations above the background. Post-hoc pairwise comparisons were performed using Dunnett's Multiple Comparison Test. Differences were considered significant when p<0.05. The statistical packages used were R 2.15 (http://www.cran.org), and GraphPad Prism version 5.04 (GraphPad Software, Inc.).

Bioinformatic Identification of 4-OT-Like Proteins.

The NCBI RefSeq database was queried using BLASTp and delta-BLAST [14] using default parameters with the S. aureus 4-OT enzyme (YP_040781.1) as a query. Further searches were performed using distant hits and results pooled, and then filtered using custom R scripts to include hits encoding proteins of 55 to 85 amino acids. Manual curation was performed, and the sequence start of predicted proteins was trimmed to begin with MP, as a proline is present in position 2 of all canonical family members [21], i.e. any amino acids purported to originate from upstream initiator codons were removed. A single sequence was selected per genus; using genus-specific sequences, an alignment was prepared using the NCBI Cobalt multiple-alignment engine [35] with default parameters. Additionally, a tree was constructed using PhyML [36] using default parameters, and visualised using Archeopterix [37] software.

Presentation of Crystal Structures

Crystal structures of proteins of interest were downloaded from the Protein data bank. A single hexameric structure was isolated from each set of crystal data using Pymol v.1.8.2 for Windows. For comparison of multiple 4-OT crystals, structures were aligned using CEAlign (Pymol) using default parameters. Pymol was also used to render images.

### Example 2: Selection of Scaffolding Tags

Study of crystal structures within the Protein Data Bank revealed a number of bacterial proteins which form self-multimers of various orders. A subset was selected based on absence of intra- or inter-chain disulphide bonding, absence of transmembrane regions and absence of toxicity and oncogenic activity, with a view to increasing the probability of efficient expression.

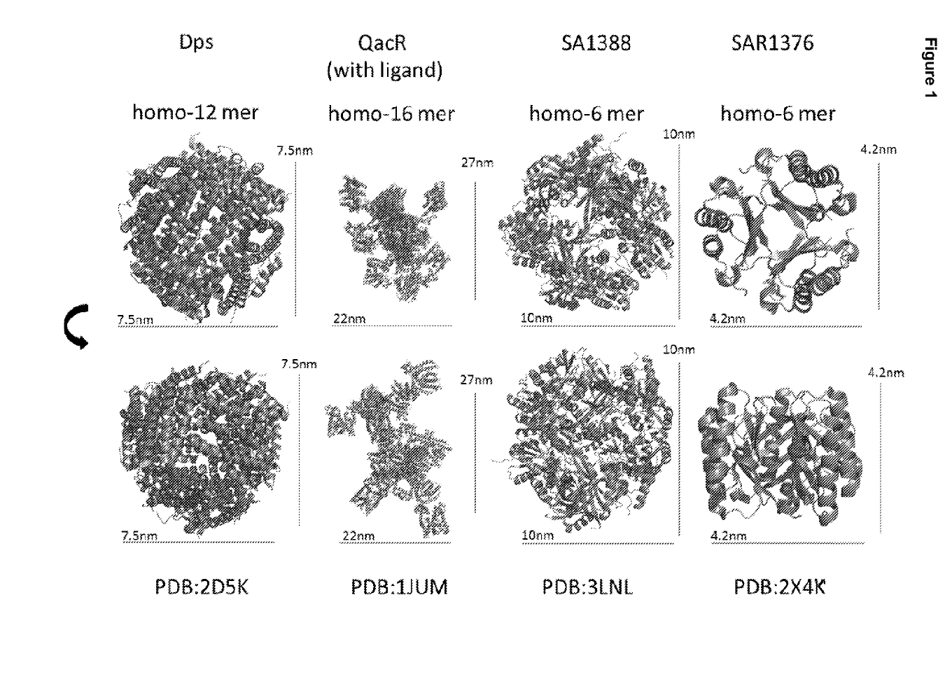

In the first instance, a number of self-multimerising proteins were selected from S. aureus, a pathogenic microbe causing disease that is controlled by both T-cell and antibody-mediated mechanisms [17]. FIG. 1 shows the structures of the four proteins which were chosen: Dps-like Peroxide Resistance Protein Dpr, with a structure similar to that of ferritin [12]; QacR, a multidrug binding transcriptional repressor; SA138818, a protein of unknown function annotated as a homologue of E. coli ygbL aldolase class 2-like gene; and SAR1376, a 4-oxalocrotonate tautomerase (4-OT). The size of the crystallising unit varied from more than 20 nm (for QacR) to less than 5 nm (for SAR1376) (FIG. 1).

### Example 3: Fusion of SAR1376 to S. aureus Antigens Enhances their Immunogenicity in Mice

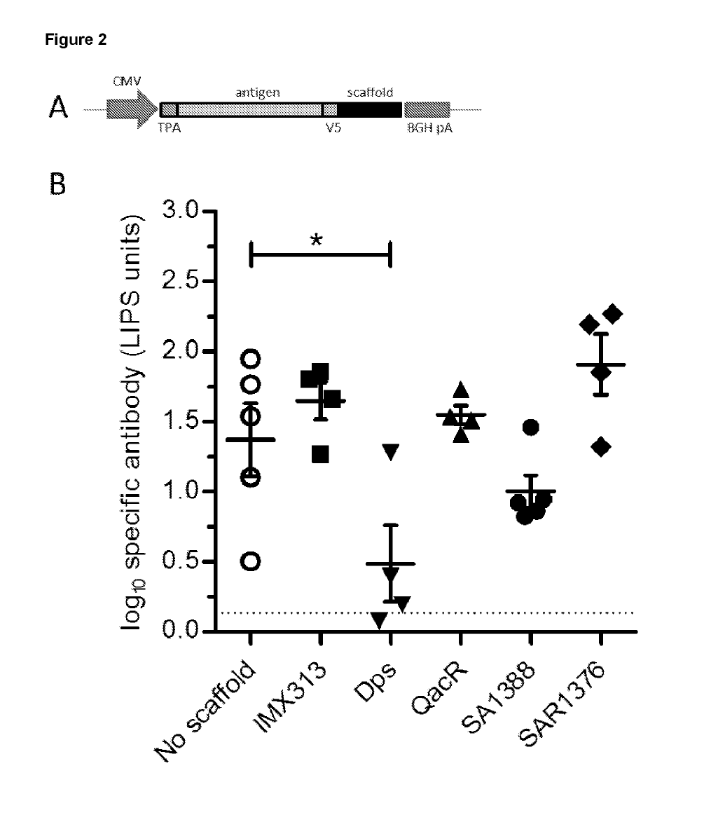

Expression vectors producing fusions of the four proteins (see Sequences herein) with a series of S. aureus antigens were constructed. The expression cassette was composed of a human tissue plasminogen activator leader sequence, the antigen of interest, an epitope tag (V5) used to monitor protein expression, and the scaffolding domain via a GSG linker (FIG. 2A).

We studied scaffold fusions to two S. aureus antigens: BitC [19], a cell surface lipoprotein (accession NP_370379), and the extracellular domain of the Clumping factor B precursor [20] (ClfB, accession YP_001333563). As comparators, constructs expressing antigen without scaffold were constructed (FIG. 2A). Additionally, in some experiments, the multimerising tag IMX313 [2] fused to the antigen C-terminus was used as a positive control.

Groups of BALB/c mice were immunised intramuscularly with a mammalian expression DNA vector expressing BitC fused to one of the four test scaffolding domains. A priming and boosting immunisation was administered, separated by two weeks (FIG. 2). Measurement of the antibody response against BitC three weeks post-boost indicated that fusion of SAR1376 might increase immunogenicity of BitC relative to the no-scaffold comparator (p=0.16), while the mice vaccinated with the Dps scaffold responded poorly against BitC (FIG. 2B).

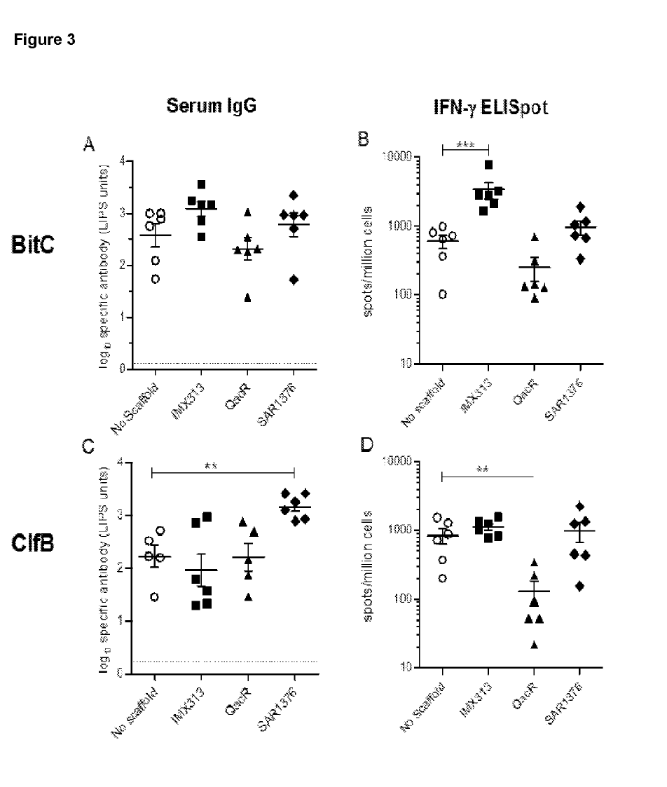

We further tested the SAR1376 and QacR scaffolds with both ClfB S. aureus antigen and with BitC, in the same 2-week prime-boost vaccination regimen. Humoral and cellular (IFN-γ ELISpot) responses were measured following the second immunisation. Comparator constructs containing the QacR domain were included in order to assess the specificity of the observed response. The immunogenicity results supported the previous experiment, in which a small increase was observed in antibody responses to BitC but not QacR (FIG. 3A; p=0.06 for BitC-SAR1376 when pooling data from experiments shown in FIGS. 2 and 3). A significantly higher antibody response to ClfB-SAR1376 was observed compared to ClfB without a scaffold (p=0.002, FIG. 3C). The SAR1376 scaffold did not enhance IFN-γ producing T-cell numbers with either of these two antigens (FIGS. 3B and 3D), although the IMX313 tag, used here as a positive control, did increase the T cell responses to BitC (FIG. 3B, p=0.01).

### Example 4: The 4-OT Family

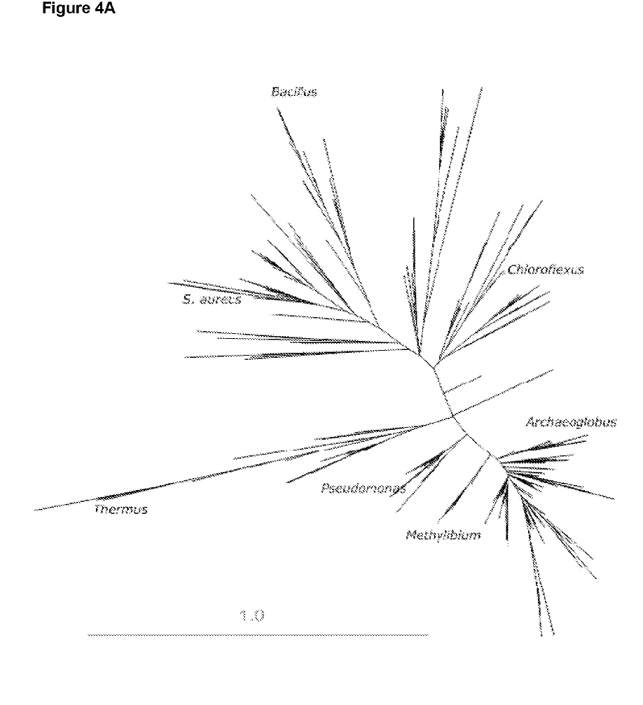

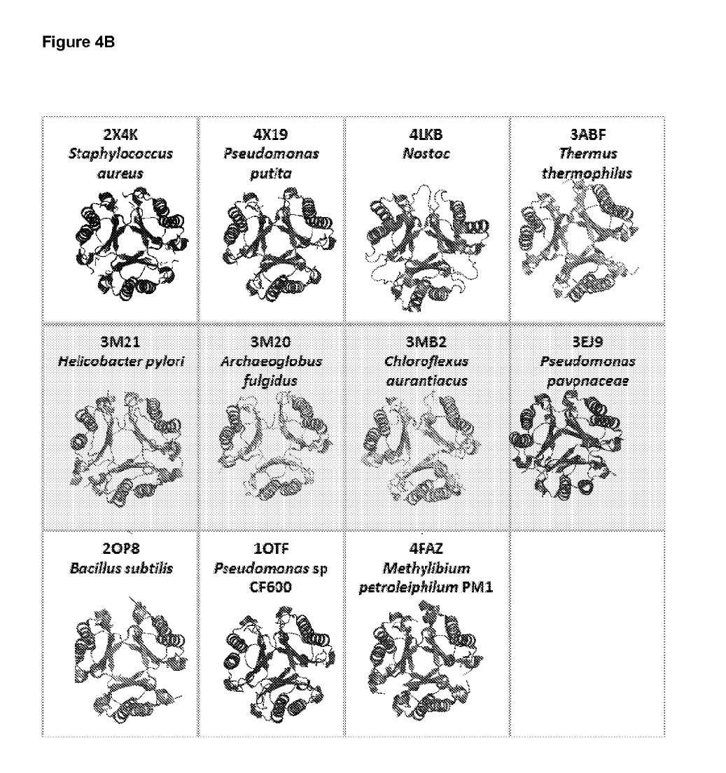

4-OT-like enzymes are common in bacteria [21]. Using a protein-based search strategy, 2780 discrete family members (modal length of 63 amino acids) were found across Eubacteria, with examples in Archaea also noted (see Example 1). One example was chosen randomly from the 342 different genera identified. Extensive diversity is observed within the protein family, with only 20% identity in primary protein sequences between diverse members of the family (FIG. 4A). Examination of eleven bacterial crystal structures, however, showed a very similar crystal structures in family members despite huge evolutionary distance (FIG. 4B). Conserved motifs, including a highly conserved initial proline, do exist within the primary sequences from genera known to be pathogenic to man (FIG. 4C).

The crystal structure of SAR1376 reveals a hexamer forming an approximately spherical structure of about 5 nm diameter. It further suggests that the amino terminus of a short linker attached to the N-terminus of SAR1376 is surface accessible. Three such amino termini are present on each side of the sphere. This suggests a model in which fusion of antigens to the N-terminus of SAR1376 generates a small sphere with six antigens displayed outwards (FIG. 1).

### Example 5: Inactivated SAR1376 Enzyme Retains the Adjuvant Activity

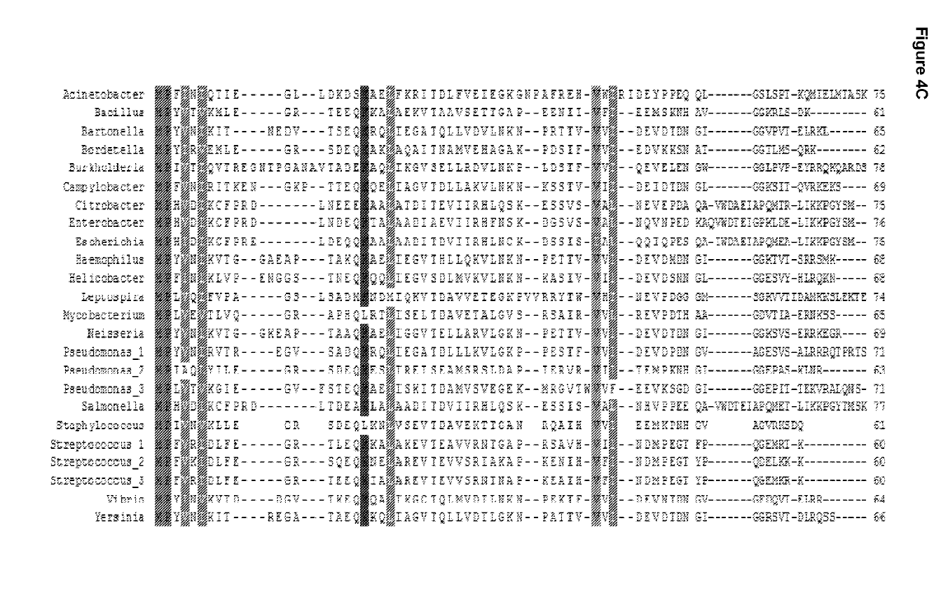

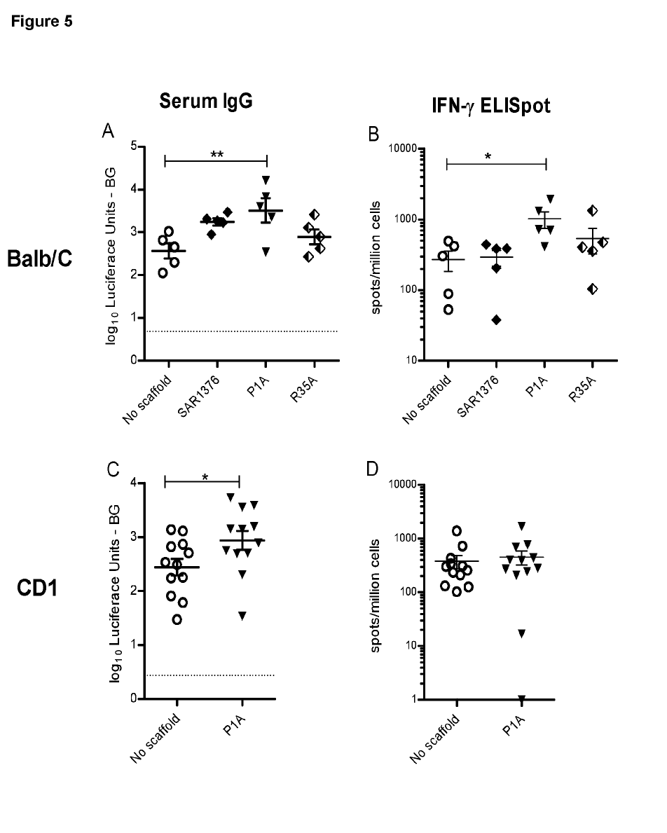

Since the mechanism of 4-OT catalysis has been heavily investigated, we mutated two critical residues involved in the active site: proline-1 (P1) and arginine-35 (R35), a site corresponding to R39 in other crystallised family members [21]. P1A mutations disrupt enzymatic activity, but leave the protein structure intact, whereas R39A or Q mutations disrupt catalysis and impair protein multimerisation [21]. The immunogenicity of ClfB fused to these variants was compared (FIG. 5). The SAR1376 mutant P1A enhanced both the antibody and T-cell responses to ClfB significantly compared to no scaffold (p=0.021) (FIGS. 5A and 5B). By contrast, the immune response to ClfB with the scaffold containing the R35A mutation [21] was not significantly different from ClfB without a scaffold (FIGS. 5A and 5B). The effect of SAR1376 mutant P1A on the ClfB antibody response was similar in both BALB/c (FIG. 5A) and outbred CD1 mice (FIG. 5C). Enhancement on IFN-γ producing T-cell numbers by P1A was observed in one mouse strain (FIGS. 5B and 5D). Taken together, this suggests that protein multimerisation contributes to the enhanced immunogenicity, but catalytic activity does not.

### Example 6: SAR1376 and the PIA Variant are Immunogenic

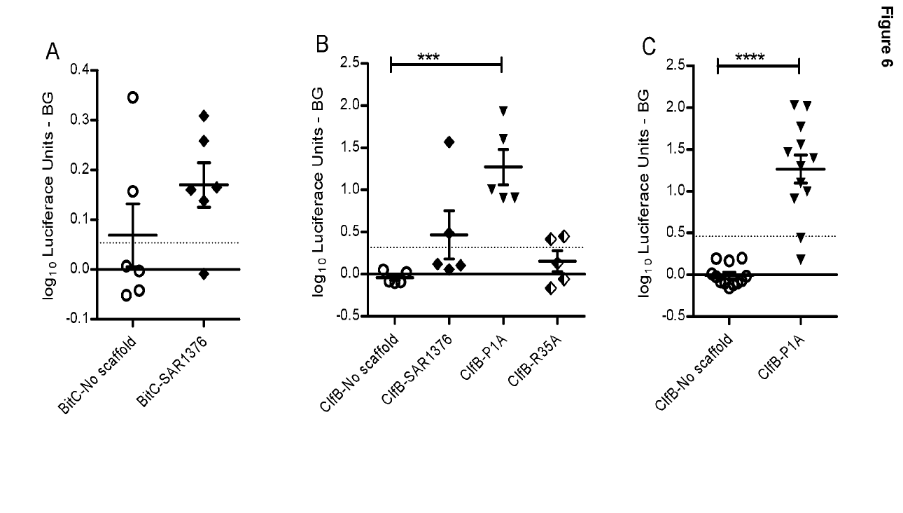

We next investigated the capacity of SAR1376 fusion proteins to raise a humoral response against SAR1376 itself. Antibody responses against SAR1376 fused to either BitC (FIG. 6A) or ClfB (FIG. 6B) in immunised BALB/c mice were not significantly raised, as compared to the no-scaffold groups. By contrast, immunization with ClfB fusion constructs containing the SAR1376 mutant PIA scaffold raised a significant antibody response to SAR1376 in both BALB/c (FIG. 6B) and CD1 outbred (FIG. 6C) mice whereas the R35A mutation did not (FIG. 6B). In both strains, serological responses against SAR1376 in the absence of SAR1376 immunisation were indistinguishable from the assay background (FIG. 6), suggesting that SAR1376 homologues, if present in colonising murine microbiota, do not generate detectable cross reactivity with SAR1376.

In summary, fusion of two S. aureus antigens to SAR1376-PIA significantly increased antibody responses to the antigens, when expressed from DNA vaccine vectors. The enhancement was observed in two different mouse strains, and was abrogated by a mutation known to disrupt multimerisation of SAR1376. Baseline immune responses against SAR1376 were not detected in the two mouse strains, but SAR1376-P1A variant is itself immunogenic.

### Example 7: Viral Vectored Vaccines Expressing SAR1376-PIA Fused to Truncated HIa Increase Immunogenicity

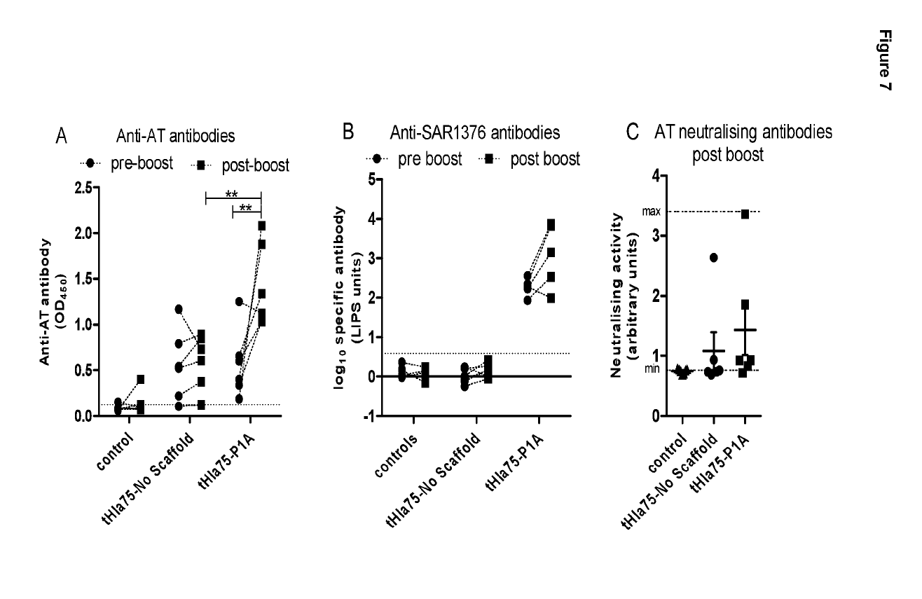

We investigated whether the pro-immunogenic effect of SAR1376 fusion was restricted to DNA vaccination. Because the effect of SAR1376 fusion appeared most marked on antibody induction, we elected to study the S. aureus alpha toxin (AT), a haemolytic multimeric β-pore forming toxin which is a critical virulence factor in S. aureus [22] and is encoded by the H/a gene. AT can be neutralised by antibody [22]. We designed a truncated form of AT, designated tHIa75, comprising amino acids 1-75, the portion of the molecule reported to contain the receptor ADAM10 (A disintegrin and metalloproteinase 10) binding domain [23, 24]. Recombinant adenoviral (AdH5) and MVA vectors expressing SAR1376 fused to tHIa75 were constructed. BALB/c mice were vaccinated with AdH5-tHIa75 followed eight weeks later by MVA-tHIa, a prime-boost regime known to be highly immunogenic [2]. Analysis of the immune response against alpha toxin showed that SAR1376 fusion did not increase the immunogenicity of adenovirally expressed proteins (FIG. 7A), but two weeks post MVA boost, a significantly-enhanced antibody response was observed in the tHIa75-PIA group relative to the tHIa75-no scaffold group (FIG. 7A). However, the tHIa75 construct did not induce substantial functional (neutralising) antibody titres (FIG. 7C) in either configuration.

As expected, antibodies against SAR3176 were raised and boosted in tHIa75-PIA vaccinated group animals (FIG. 7B).

### Example 8: Fusion of SAR1376-PIA to Pfs25 Recombinant Protein Improves Immunogenicity in BALB/c Mice

We investigated whether the pro-immunogenic effect of SAR1376 fusion extended to recombinant protein antigens by studying the effect SAR1376 fusion on immunogenicity of a P. falciparum protein, Pfs25. Pfs25 is a candidate antigen for a transmission blocking vaccine and antibodies against Pfs25 have been shown in several studies to interfere with sexual reproduction of the parasite in the mosquito vector [25]. Recently, we have reported that heptamerisation of Pfs25 by fusion to IMX313 increased its immunogenicity significantly in pre-clinical studies [10].

Recombinant monomeric Pfs25 and Pfs25-SAR1376-P1A proteins were produced in Pichia pastoris as secreted proteins and purified using a 6-Histidine tag (FIGS. 8A and 8B). Mice were immunised twice with either Pfs25-SAR1376-P1A or monomeric Pfs25 protein-in-Alhydrogel formulations at 2-week intervals.

Little response was seen in mice following vaccination with Pfs25. The antibody levels at all time points were significantly higher in the group that received Pfs25-SAR1376-P1A than the mice receiving monomeric Pfs25, demonstrating that fusion of Pfs25 to SAR1376-P1A significantly improved the immune response (FIG. 8C). The pooled and purified IgG from the Pfs25-SAR1376-P1A immunised mice (day 28) were able to completely block oocyst development in the mosquito midgut in the ex vivo Standard Membrane Feeding Assay (SMFA), demonstrating that antibodies produced by immunisation with this platform were functional (FIG. 8D).

### REFERENCES

1 De Gregorio, E., Caproni, E. & Ulmer, J. B. Vaccine adjuvants: mode of action. Front Immunol 4, 214, doi:10.3389/fimmu.2013.00214 (2013).
2 Rollier, C. S., Reyes-Sandoval, A., Cottingham, M. G., Ewer, K. & Hill, A. V. Viral vectors as vaccine platforms: deployment in sight. Curr Opin Immunol 23, 377-382, doi:10.1016/j.coi.2011.03.006 (2011).
3 Tissot, A. C. et al. Versatile virus-like particle carrier for epitope based vaccines. PLoS One 5, e9809, doi:10.1371/journal.pone.0009809 (2010).
4 Bachmann, M. F. & Jennings, G. T. Vaccine delivery: a matter of size, geometry, kinetics and molecular patterns. Nature reviews. Immunology 10, 787-796, doi:10.1038/nri2868 (2010).
5 Lopez-Sagaseta, J., Malito, E., Rappuoli, R. & Bottomley, M. J. Self-assembling protein nanoparticles in the design of vaccines. Comput Struct Biotechnol J 14, 58-68, doi:10.1016/j.csbj.2015.11.001 (2016).
6 Lee, L. A. & Wang, Q. Adaptations of nanoscale viruses and other protein cages for medical applications. Nanomedicine 2, 137-149, doi:10 1016/j.nano.2006.07.009 (2006).
7 Richardson, C., Bargatze, R. F., Goodwin, R. & Mendelman, P. M. Norovirus virus-like particle vaccines for the prevention of acute gastroenteritis. Expert review of vaccines 12, 155-167, doi:10.1586/erv.12.145 (2013).
8 Da Silva, D. M. et al. Physical interaction of human papillomavirus virus-like particles with immune cells. International Immunology 13, 633-641, doi:10.1093/intimm/13.5.633 (2001).
9 Al-Barwani, F., Young, S. L., Baird, M. A., Larsen, D. S. & Ward, V. K. Mannosylation of virus-like particles enhances internalization by antigen presenting cells. PLoS One 9, e104523, doi:10.1371/journal.pone.0104523 (2014).
10 Li, Y. et al. Enhancing immunogenicity and transmission-blocking activity of malaria vaccines by fusing Pfs25 to IMX313 multimerization technology. Sci Rep 6, 18848, doi:10.1038/srep18848 (2016).
11 Forbes, E. K. et al. T cell responses induced by adenoviral vectored vaccines can be adjuvanted by fusion of antigen to the oligomerization domain of C4b-binding protein. PLoS One 7, e44943, doi:10.1371/journal.pone.0044943 (2012).
12 Kanekiyo, M. et al. Rational Design of an Epstein-Barr Virus Vaccine Targeting the Receptor-Binding Site. Cell 162, 1090-1100, doi:10.1016/j.cell.2015.07.043 (2015).
13 Jardine, J. et al. Rational HIV immunogen design to target specific germline B cell receptors. Science 340, 711-716, doi:10.1126/science.1234150 (2013).
14 Boratyn, G. M. et al. Domain enhanced lookup time accelerated BLAST. Biology direct 7, 12, doi:10.1186/1745-6150-7-12 (2012).
15 Kaufmann, S. H. Tuberculosis vaccine development ata divide. Current opinion in pulmonary medicine 20, 294-300, doi:10.1097/MCP.0000000000000041 (2014).
16 White, M. T. et al. A combined analysis of immunogenicity, antibody kinetics and vaccine efficacy from phase 2 trials of the RTS,S malaria vaccine. BMC medicine 12, 117, doi:10 1186/PREACCEPT-5212460251240508 (2014).
17 Daum, R. S. & Spellberg, B. Progress toward a Staphylococcus aureus vaccine. Clin Infect Dis 54, 560-567, doi:10.1093/cid/cir828 (2012).
18 Saikatendu, K. S. et al. Structure of a conserved hypothetical protein SA1388 from S. aureus reveals a capped hexameric toroid with two PII domain lids and a dinuclear metal center. BMC structural biology 6, 27, doi:10.1186/1472-6807-6-27 (2006).
19 Dugourd, D., Martin, C., Rioux, C. R., Jacques, M. & Harel, J. Characterization of a periplasmic ATP-binding cassette iron import system of Brachyspira (Serpulina) hyodysenteriae. J Bacteriol 181, 6948-6957 (1999).
20 Ni Eidhin, D. et al. Clumping factor B (ClfB), a new surface-located fibrinogen-binding adhesin of Staphylococcus aureus. Molecular microbiology 30, 245-257 (1998).
21 Whitman, C. P. The 4-oxalocrotonate tautomerase family of enzymes: how nature makes new enzymes using a beta-alpha-beta structural motif. Archives of biochemistry and biophysics 402, 1-13, doi:10.1016/S0003-9861(02)00052-8 (2002).
22 Sampedro, G. R. et al. Targeting Staphylococcus aureus alpha-toxin as a novel approach to reduce severity of recurrent skin and soft-tissue infections. J Infect Dis 210, 1012-1018, doi:10.1093/infdis/jiu223 (2014).
23 Inoshima, I. et al. A Staphylococcus aureus pore-forming toxin subverts the activity of ADAM10 to cause lethal infection in mice. Nat Med 17, 1310-1314, doi:10.1038/nm.2451 (2011).
24 Wilke, G. A. & Bubeck Wardenburg, J. Role of a disintegrin and metalloprotease 10 in Staphylococcus aureus alpha-hemolysin-mediated cellular injury. Proc Natl Acad Sci USA 107, 13473-13478, doi:10.1073/pnas.1001815107 (2010).
25 Kaslow, D. C. et al. A vaccine candidate from the sexual stage of human malaria that contains EGF-like domains. Nature 333, 74-76, doi:10.1038/333074a0 (1988).
26 Huddleston, J. P., Burks, E. A. & Whitman, C. P. Identification and characterization of new family members in the tautomerase superfamily: analysis and implications. Archives of biochemistry and biophysics 564, 189-196, doi:10.1016/j.abb.2014.08.019 (2014).
27 4-Oxalocrotonate like proteins: Uniprot query, <http://www.uniprot.org/uniprot/?query=family:%224-oxalocrotonate+tautomerase+family %22> (2014).
28 Ewer, K. J. et al. Viral vectors as vaccine platforms: from immunogenicity to impact. Curr Opin Immunol 41, 47-54, doi:10.1016/j.coi.2016.05.014 (2016).
29 Gilbert, S. C. et al. Enhanced CD8 T cell immunogenicity and protective efficacy in a mouse malaria model using a recombinant adenoviral vaccine in heterologous prime-boost immunisation regimes. Vaccine 20, 1039-1045 (2002).
30 Draper, S. J. et al. Effective induction of high-titer antibodies by viral vector vaccines. Nat Med 14, 819-821, doi:10.1038/nm.1850 (2008).
31 Burbelo, P. D., Goldman, R. & Mattson, T. L. A simplified immunoprecipitation method for quantitatively measuring antibody responses in clinical sera samples by using mammalian-produced Renilla luciferase-antigen fusion proteins. BMC biotechnology 5, 22, doi:10.1186/1472-6750-5-22 (2005).
32 van Diemen, P. M. et al. Irradiated wild-type and Spa mutant Staphylococcus aureus induce anti-S. aureus immune responses in mice which do not protect against subsequent intravenous challenge. Pathog Dis 68, 20-26, doi:10.1111/2049-632X.12042 (2013).
33 Oscherwitz, J. & Cease, K. B. Identification and validation of a linear protective neutralizing epitope in the beta-pore domain of alpha toxin. PLoS One 10, e0116882, doi:10.1371/journal.pone.0116882 (2015).
34 Miura, K. et al. Qualification of standard membrane-feeding assay with Plasmodium falciparum malaria and potential improvements for future assays. PLoS One 8, e57909, doi:10.1371/journal.pone.0057909 (2013).
35 Papadopoulos, J. S. & Agarwala, R. COBALT: constraint-based alignment tool for multiple protein sequences. Bioinformatics 23, 1073-1079, doi:10.1093/bioinformatics/btm076 (2007).
36 Guindon, S. et al. New algorithms and methods to estimate maximum-likelihood phylogenies: assessing the performance of PhyML 3.0. Systematic biology 59, 307-321, doi:10.1093/sysbio/syq010 (2010).
37 Creech, C. B., 2nd et al. Vaccination as infection control: a pilot study to determine the impact of Staphylococcus aureus vaccination on nasal carriage. Vaccine 28, 256-260, doi:50264-410X(09)01435-2 [pii] 10.1016/j.vaccine.2009.09.088 (2009).

SEQUENCES

SEQ ID NO: 1
Acinetobacter_baumannii_OIFC021 gi444755286
MPFINIQTIEGLLDKDSKAELFKRITDLFVEIEGKGNPAFREHVWIRIDEYPPEQWQLGSLSPTKQMIELMTASK
SEQ ID NO: 2
Bacillus_cereus gi487908138
MPYVTVKMLEGRTEEQKKALAEKVTAAVSETTGAPEENIIVFIEEMSKNHYAVGGKRLSDK
SEQ ID NO: 3
Bartonella_elizabethae_Re6043vi gi395417463
MPYVNIKITNEDVTSEQKRQLIEGATQLLVDVLNKNPRTTVVVIDEVDTDNWGIGGVPVTELRKL
SEQ ID NO: 4
Bordetella_sp._FB-8 gi518780346
MPYIRVEMLEGRSDEQKAKLAQAITNAMVEHAGAKPDSIFVVIEDVKKSNWATGGILMSQRK
SEQ ID NO: 5
Burkholderia_sp._RPE67 gi636800047
MPIVTIQVTREGNTPGANAVTADEKAQLIKGVSELLRDVLNKPLDSTFVVIQEVELENWGWGGLPVPEYRRQKQARD
S
SEQ ID NO: 6
Campylobacter_jejuni gi657884310
MPFVNIRITKENGKPTTEQKQELIAGVTDLLAKVLNKNKSSTVVIIDEIDTDNYGLGGKSITQVRKEKS
SEQ ID NO: 7
Citrobacter_freundii gi489932881
MPHVDIKCFPRDLNEEEKAALATDITEVIIRHLQSKESSVSVALNEVEPDAWQAVWDAEIAPQMTRLIKKPGYSM
SEQ ID NO: 8
Enterobacter_cloacae gi654545569
MPHVDIKCFPRDLNDEQKTALAADIAEVIIRHFNSKDGSVSVALNQVNPEDWKAQVWDTEIGPKLDELIKKPGYSM
SEQ ID NO: 9
Escherachia_coli gi447042883
MPHIDIKCEPRELDEQQKAALAADITDVIIRHLNCKDSSISIALQQIQPESWQAIWDAEIAPQMEALIKKPGYSM
SEQ ID NO: 10
Haemophilus_parainfluenzae gi503830159
MRYVNIKVTGGAEAPTAKQKAELIEGVTHLLQKVLNKNPETTVVVIDEVDMDNWGIGGKTVTSRRSMK
SEQ ID NO: 11
Helicobacter_pylori gi639858229
MPFINIKLVPENGGSTNEQKQQLIEGVSDLMVKVLNKNKASIVVIIDEVDSNNYGLGGESVYHLRQKN
SEQ ID NO: 12
Leptospira_interrogans_serovar_Djasiman_str._LT1649 gi464164388
MPLVQIFVPAGSLSADMKNDMIQKVTDAVVETEGKPVVRRYTWVHINEVPDGGWGMSGKVVTIDAMKKSLEKTE
SEQ ID NO: 13
Mycobacterium_intracellulare gi497639745
MPLVEVTLVQGRAPHQLRTLISELTDAVETALGVSRSAIRVVLREVPDTHWAAGDVTIAERNKSS
SEQ ID NO: 14
Neisseria_meningitidis gi488141741
MPYVNIKVTGGKEAPTAAQKAELIGGVTELLARVLGKNPETTVVVIDEVDTDNWGIGGKSVSERRKEGR
SEQ ID NO: 15
Neisseria_gonorrhoeae gi636860961
MPYVNIKVTGGKEAPTAAQKAELIGGVIELLARVLGKNPETTVVVIDEVDTDNWDIGGKSVSERRKEGR
SEQ ID NO: 16
Pseudomonas_aeruginosa gi501023324
MPYVNIRVTREGVSADQKRQLIEGATDLLLKVLGKPPESTFVVIDEVDPDNWGVAGESVSALRRRQTPRTS
SEQ ID NO: 17
Pseudomonas_putida gi639678172
MPIAQLYILEGRSDEQKESLIREISEAMSRSLDAPIERVRVIITEMPKNHFGIGGEPASKLNR
SEQ ID NO: 18
Pseudomonas_aeruginosa gi489251658
MPLVTVKGIEGVFSTEQKAEIISKITDAMVSVEGEKMRGVTWVVFEEVKSGDWGIGGEPITTEKVRALQNS
SEQ ID NO: 19
Salmonella_enterica gi555267960
MPHVDIKCFPRDLTDEAKLALAADITDVIIRHLQSKESSISVALNHVPPEEWQAVWDTEIAPQMETLIKKPGYTMSK
SEQ ID NO: 20
Staphylococcus_aureus gi447046020
MPIVNVKLLEGRSDEQLKNLVSEVTDAVEKTTGANRQAIHVVIEEMKPNHYGVAGVRKSDQ
SEQ ID NO: 21
Streptococcus_pneumoniae gi642960263
MPEVRIDLFEGRTLEQKKALAKEVTEAVVRNTGAPRSAVHVIINDMPEGTYFPQGEMRTK
SEQ ID NO: 22
Streptococcus_agalactiae gi657924480
MPFVKIDLFEGRSQEQKNELAREVTEVVSRIAKAPKENIHVFINDMPEGTYYPQDELKKK
SEQ ID NO: 23
Streptococcus_suis gi489024563
MPFVRIDLFEGRTEEQKIALAREVTEVVSRNTNAPKEAIHVFINDMPEGTYYPQGEMKRK
SEQ ID NO: 24
Vibrio_fluvialis gi520908977
MPYINVKVTDDGVTKEQKQAIIKGCTQLMVDILNKNPEKTFVVIDEVNTDNWGVGFDQVTELRR
SEQ ID NO: 25
Yersinia_pseudotuberculosis gi501054428
MPYVNIKITREGATAEQKKQLIAGVTQLLVDTLGKNPATTVVVIDEVDTDNWGIGGRSVTDLRQSS
SEQ ID NO: 26
Yersinia_enterocolitica gi644933472
MPYVNIKITREGATAEQKKQLIAGVTQLLVDTLGKNPATTVVVIDEVDTDNWGIGGHSVTELRKSS
SEQ ID NO: 27
SAR1376 (amino acid sequence)
MMPIVNVKLLEGRSDEQLKNLVSEVTDAVEKTTGANRQAIHVVIEEMKPNHYGVAGVRKSDQ
SEQ ID NO: 28
SAR1376 P1A (amino acid sequence, mutation underlined)
MMAIVNVKLLEGRSDEQLKNLVSEVTDAVEKTTGANKAIHVVIEEMKPNHYGVAGVRKSDQ
SEQ ID NO: 29
SAR1376 R35A (amino acid sequence, mutation underlined)
MMPIVNVKLLEGRSDEQLKNLVSEVTDAVEKTTGANAQAIHVVIEEMKPNHYGVAGVRKSDQ
SEQ ID NO: 30
SAR1376
ATGATGCCCATCGTGAACGTGAAGCTGCTGGAAGGCAGAAGCGACGAGCAGCTGAAGAACCTGGTGTCCGA
AGTGACCGACGCCGTGGAAAAGACCACCGGCGCCAACAGACAGGCCATCCACGTCGTGATCGAGGAAATGAAGCCCA
ACCACTACGGCGTGGCCGGCGTGCGGAAAAGCGATCAGTGATGA
SEQ ID NO: 31
SAR1376 P1A (mutation underlined)
ATGATGGCCATCGTGAACGTGAAGCTGCTGGAAGGCAGAAGCGACGAGCAGCTGAAGAACCTGGTGTCCGA
AGTGACCGACGCCGTGGAAAAGACCACCGGCGCCAACAGACAGGCCATCCACGTCGTGATCGAGGAAATGAAGCCCA
ACCACTACGGCGTGGCCGGCGTGCGGAAAAGCGATCAGTGATGA
SEQ ID NO: 32
SAR1376 R35A (mutation underlined)
ATGATGCCCATCGTGAACGTGAAGCTGCTGGAAGGCAGAAGCGACGAGCAGCTGAAGAACCTGGTGTCCGA
AGTGACCGACGCCGTGGAAAAGACCACCGGCGCCAACGCCCAGGCCATCCACGTCGTGATCGAGGAAATGAAGCCCA
ACCACTACGGCGTGGCCGGCGTGCGGAAAAGCGATCAGTGATGA
SEQ ID NO: 33
SA1388
ATGAAGATCGCCGACCTGATGACCCTGCTGGACCACCACGTGCCCTTTAGCACAGCCGAGAGCTGGGACAA
CGTGGGCCTGCTGATTGGCGACGGCGACGTGGAAGTGACCGGCGTGCTGACAGCCCTGGACTGCACACTGGAAGTCG
TGAACGAGGCCATCGAGAAGGGCTACAACACCATCATCAGCCACCACCCCCTGATCTTCAAGGGCGTGACCAGCCTG
AAGGCCAACGGCTACGGCCTGATCATCCGGAAGCTGATCCAGCACGACATCAACCTGATCGCCATGCACACCAACCT
GGACGTGAACCCCTACGGCGTGAACATGATGCTGGCCAAGGCCATGGGCCTGAAGAACATCAGCATCATCAACAACC
AGCAGGACGTGTACTACAAGGTGCAGACCTACATCCCCAAGGATAATGTGGGCCCCTTCAAGGACAAGCTGAGCGAG
AATGGCCTGGCCCAGGAAGGCAACTACGAGTACTGCTTCTTCGAGAGCGAGGGCAGAGGCCAGTTCAAGCCTGTGGG
CGAGGCCAACCCTACCATCGGCCAGATCGACAAGATCGAGGACGTGGACGAAGTGAAGATCGAGTTCATGATCGACG
CCTACCAGAAGTCCAGAGCCGAGCAGCTGATCAAGCAGTACCACCCCTACGAGACACCCGTGTTCGACTTCATCGAG
ATTAAGCAGACCTCCCTGTACGGCCTGGGCGTGATGGCCGAGGTGGACAACCAGATGACTCTGGAAGATTTCGCCGC
CGACATCAAGAGCAAGCTGAACATCCCCTCCGTCAGATTCGTGGGCGAGAGCAACCAGAAGATCAAGCGGATCGCCA
TCATCGGCGGCAGCGGCATCGGCTACGAGTATCAGGCTGTGCAGCAGGGCGCCGACGTGTTCGTGACAGGCGATATC
AAGCACCACGACGCCCTGGACGCCAAGATCCATGGCGTGAACCTGATCGACATCAACCACTACAGCGAGTACGTGAT
GAAGGAAGGCCTGAAAACCCTGCTGATGAACTGGTTCAATATCGAGAAGATTAACATTGATGTGGAAGCCAGCACCA
TCAATACCGACCCCTTCCAGTACATCTGATGATGA
SEQ ID NO: 34
Dps
ATGAGCAACCAGCAGGACGTCGTGAAAGAACTGAATCAGCAGGTGGCCAACTGGACCGTGGCCTACACCAA
GCTGCACAACTTCCATTGGTACGTGAAGGGCCCCAACTTCTTCAGCCTGCACGTGAAGTTCGAGGAACTGTACAACG
AGGCCAGCCAGTACGTGGACGAGCTGGCCGAGAGAATCCTGGCCGTGGGCGGAAATCCTGTGGGCACCCTGACCGAG
TGCCTGGAACAGAGCATTGTGAAAGAGGCCGCCAAGGGCTACAGCGCCGAGCAGATGGTGGAAGAACTGAGCCAGGA
CTTCACCAACATCAGCAAGCAGCTGGAAAACGCCATCGAGATCGCCGGCAACGCTGGCGACGATGTGTCCGAGGACA
TGTTCATCGGCATGCAGACCAGCGTGGACAAGCACAACTGGATGTTCAAGAGCTACCTGAGCTGATGATGA
SEQ ID NO: 35
QacR
ATGAACCTGAAGGACAAGATCCTGGGCGTGGCCAAAGAGCTGTTCATCAAGAACGGCTACAACGCCACCAC
CACCGGCGAGATCGTGAAGCTGAGCGAGAGCAGCAAGGGCAACCTGTACTACCACTTCAAGACCAAAGAGAACCTGT
TCCTGGAAATCCTGAACATCGAGGAATCCAAGTGGCAGGAACAGTGGAAGAAAGAACAGATCAAGTGCAAGACCAAC
CGCGAGAAGTTCTACCTGTACAACGAGCTGAGCCTGACCACCGAGTACTACTACCCCCTGCAGAACGCCATCATCGA
GTTCTGCACAGAGTACTACAAGACCAATAGCATCAACGAGAAGATGAACAAGCTGGAAAACAAGTACATCGACGCCT
ACCACGTGATCTTCAAAGAGGGCAATCTGAACGGCGAGTGGTGCATCAATGACGTGAACGCCGTGTCCAAGATCGCC
GCCAACGCCGTGAATGGCATCGTGACCTTCACCCACGAGCAGAACATCAATGAGCGGATCAAGCTGATGAACAAATT
CAGCCAGATCTTCCTGAACGGCCTGAGCAAGTGATGA

1. A fusion polypeptide, wherein the fusion polypeptide comprises:
(a) a 4-oxalocrotonate tautomerase (4-OT)-based polypeptide scaffold which is capable of forming multimers; and
(b) a polypeptide antigen.

2. A fusion polypeptide as claimed in claim 1, wherein the 4-OT based polypeptide scaffold is from or derived from a bacterium of a genera selected from the group consisting of Acinetobacter, Bacillus, Bartonella, Bordetella, Burkholderia, Campylobacter, Citrobacter, Enterobacter, Escherichia, Haemophilus, Helicobacter, Leptospira, Mycobacterium, Neisseria, Pseudomonas, Rhodobacter, Salmonella, Staphylococcus, Streptococcus, Thermoanaerobacter, Vibrio and Yersinia.

3. A fusion polypeptide as claimed in claim 1 or claim 2, wherein 4-OT based polypeptide scaffold is from or derived from Staphylococcus aureus.

4. A fusion polypeptide as claimed in any one of claims 1 to 3, wherein the the 4-OT based polypeptide scaffold comprises or consists of an amino acid sequence as given in any one of SEQ ID NOs: 38-127, or a variant or derivative thereof having at least 50% sequence identity thereto, more preferably at least 60%, 70%, 80%, 90% or 95% sequence identity thereto, which retains the ability to multimerise and to present the polypeptide antigen.

5. A fusion polypeptide as claimed in any one of claims 1 to 3, wherein the 4-OT based polypeptide scaffold comprises or consists of an amino acid sequence as given in any one of SEQ ID NOs: 128-627, or a variant or derivative thereof having at least 80% sequence similarity thereto, more preferably at least 90% or 95% sequence similarity thereto, which retains the ability to multimerise and to present the polypeptide antigen

6. A fusion polypeptide as claimed in any one of claims 1 to 3, wherein the 4-OT based polypeptide scaffold comprises or consists of an amino acid sequence as given in any one of SEQ ID NOs: 128-627, or a variant or derivative thereof having at least 80% sequence identity thereto, more preferably at least 90% or 95% sequence identity thereto, which retains the ability to multimerise and to present the polypeptide antigen.

7. A fusion polypeptide as claimed in any one of claims 1 to 3, wherein the 4-OT based polypeptide scaffold comprises or consists of an amino acid sequence as given in any one of SEQ ID NOs: 1-29, or a variant or derivative thereof which retains the ability to multimerise and to present the polypeptide antigen.

8. A fusion polypeptide as claimed in claim 7, wherein the variant of the 4-OT based polypeptide scaffold comprises or consists of an amino acid sequence having at least 30%, 40%, 50%, 60%, 70%, 80%, 90%, 95% or 99% amino acid sequence identity to any one of SEQ ID NOs: 1-29.

9. A fusion polypeptide as claimed in claim 7, wherein the derivative of the 4-OT based polypeptide scaffold comprises or consists of a fragment of any one of SEQ ID NOs: 1-29 which is at least 70%, 80%, 90%, 95% or 99% of the length of that SEQ ID NO.

10. A fusion polypeptide as claimed in any one of the preceding claims, wherein the polypeptide antigen is one which is presented on a microbe, parasite or neoplasm.

11. A fusion polypeptide as claimed in any one of the preceding claims, wherein the polypeptide antigen is a viral, bacterial, protozoan, animal, mammalian or human antigen.

12. A fusion polypeptide as claimed in any one of the preceding claims, wherein the polypeptide antigen is from a disease-causing bacterium, a disease-causing parasite or a disease-causing virus, preferably from a malaria-causing parasite or an influenza-causing virus.

13. A fusion polypeptide as claimed in any one of the preceding claims, wherein the polypeptide antigen is a bacterial or parasitic antigen which is from or derived from Staphylococcus, pathogenic Neisseria, Mycobacteria, Escherichia or from Apicomplexa (e.g. Plasmodium) or helminths, preferably from or derived from S. aureus, a pathogenic Neisseria species, M. tuberculosis, E. coli or P. falciparum.

14. A fusion polypeptide as claimed in any one of the preceding claims, wherein the polypeptide antigen is selected from the group consisting of ClfA, ClfB, FnBPA, FnBP, SdrC, SdrD, SdrE, SasA, SasB, SasC, SasD, SasX, SasF, SasG/AAp, MntC, IsdA, IsdB, IsdH, FhuD2, EsxA, EsxB, Spa, Coa, vWbp, HIa, HIgA, HIgB, HIgC, LukA, LukB, LukD, LukE, EpiP, Can, Csa1A, Csa1B, Csa1C, Csa1D, CsA2A, Csa3A, Csa3B, CsA3C, Csa3D, Csa3E, Csa3G, Csa3H, Csa31, Csa3J, Csa4A, Csa4B, Csa4c, scn, efb, efbc, or a variant or derivative thereof which maintains the immunogenic capacity of the antigen.

15. A fusion polypeptide as claimed in any one of claims 1 to 13, wherein the polypeptide antigen is S. aureus BitC; the extracellular domain of the S. aureus Clumping factor B precursor (ClfB); the S. aureus alpha toxin or a truncated form thereof; or the P. falciparum protein Pfs25.

16. A homo-multimer, wherein the homo-multimer comprises 2, 3, 4, 5, 6, 7, 8, 9, 10, 12, 16 or 20 fusion polypeptides as defined in any one of claims 1 to 15.

17. A hetero-multimer, wherein the hetero-multimer comprises 2, 3, 4, 5, 6, 7, 8, 9, 10, 12, 16 or 20 fusion polypeptides as defined in any one of claims 1 to 15.

18. A hetero-multimer as claimed in claim 17, wherein the hetero-multimer comprises 2, 3, 4, 5 or 6 different fusion polypeptides as defined in any one of claims 1 to 15.

19. A hetero-multimer as claimed in claim 17 or claim 18, wherein the hetero-multimer comprises the same 4-OT-based polypeptide scaffold but different polypeptide antigens.

20. A nucleic acid molecule encoding a fusion polypeptide as claimed in any one of claims 1 to 15, preferably wherein the nucleic acid molecule is DNA.

21. A vector or plasmid comprising a nucleic acid molecule as claimed in claim 20, preferably wherein the vector is an expression vector.

22. A vector as claimed in claim 21, wherein the vector is a viral vector, preferably an Adenoviral vector or a Modified Vaccinia Ankara (MVA) viral vector.

23. A host cell comprising a nucleic acid molecule as claimed in claim 20 or a vector or plasmid as claimed in claim 21 or claim 22, preferably wherein the host cell is a eukaryotic cell.

24. A pharmaceutical composition comprising a fusion polypeptide as claimed in any one of claims 1 to 15, a multimer as claimed in any one of claims 16-19, a nucleic acid molecule as claimed in claim 20, or a vector or plasmid as claimed in claim 21 or 22, optionally together with one or more pharmaceutically-acceptable carriers, excipients or diluents.

25. A vaccine composition comprising a fusion polypeptide as claimed in any one of claims 1 to 15, a multimer as claimed in any one of claims 16-19, a nucleic acid molecule as claimed in claim 20, or a vector or plasmid as claimed in claim 21 or 22, optionally together with an adjuvant.

26. A combined preparation comprising two or more components selected from the group consisting of fusion polypeptide as claimed in any one of claims 1 to 15, multimers as claimed in any one of claims 16-19, nucleic acid molecules as claimed in claim 20, and vectors or plasmids as claimed in claim 21 or 22, as a combined preparation in a form suitable for simultaneous, separate or sequential use, preferably for use in producing an immunogenic response to the polypeptide antigen in a subject.

27. A fusion polypeptide as claimed in any one of claims 1 to 15, a multimer as claimed in any one of claims 16-19, a nucleic acid molecule as claimed in claim 20, a vector or plasmid as claimed in claim 21 or 22, or a composition or preparation as claimed in any one of claims 24 to 26, for use in therapy or for use as a medicament.

28. A fusion polypeptide as claimed in any one of claims 1 to 15, a multimer as claimed in any one of claims 16-19, a nucleic acid molecule as claimed in claim 20, a vector or plasmid as claimed in claim 21 or 22, or a composition or preparation as claimed in any one of claims 24 to 26, for use in producing an immunogenic response to the polypeptide antigen in a subject, preferably for inducing a T-cell or a B-cell response to the polypeptide antigen in a subject.

29. Use of a fusion polypeptide as claimed in any one of claims 1 to 15, a multimer as claimed in any one of claims 16-19, a nucleic acid molecule as claimed in claim 20, a vector or plasmid as claimed in claim 21 or 22, or a composition or preparation as claimed in any one of claims 24 to 26, in the preparation of a medicament for producing an immunogenic response to the polypeptide antigen in a subject, preferably for inducing a T-cell or a B-cell response to the polypeptide antigen in a subject.

30. A method of inducing an immunogenic response to a polypeptide antigen in a subject, the method comprising administering an effective amount of a fusion polypeptide as claimed in any one of claims 1 to 15, a multimer as claimed in any one of claims 16-19, a nucleic acid molecule as claimed in claim 20, a vector or plasmid as claimed in claim 21 or 22, or a composition or preparation as claimed in any one of claims 24 to 26, to the subject.

31. A method of inducing an immunogenic response to a polypeptide antigen in a subject, method comprising administering to the subject:
(i) an effective priming amount of a fusion polypeptide as claimed in any one of claims 1 to 15, a multimer as claimed in any one of claims 16-19, a nucleic acid molecule as claimed in claim 20, a vector or plasmid as claimed in claim 21 or 22, or a composition or preparation as claimed in any one of claims 24 to 26; and then
(ii) an effective boosting amount of a fusion polypeptide as claimed in any one of claims 1 to 15, a multimer as claimed in any one of claims 16-19, a nucleic acid molecule as claimed in claim 20, a vector or plasmid as claimed in claim 21 or 22, or a composition or preparation as claimed in any one of claims 24 to 26.

32. A method as claimed in claim 31, the method comprising administering to the subject:
(i) an effective priming amount of an adenovirus vector as claimed in claim 22 (preferably AdHu5); and then
(ii) an effective boosting amount of a non-replicating poxvirus vector as claimed in claim 22 (preferably MVA).

33. A process for the production of one or more of fusion polypeptides as claimed in any one of claims 1 to 15, which process comprises expressing one or more nucleic acid molecules as claimed in claim 20 in a host cell, and recovering the fusion polypeptide product(s).
